(19) United States
(12) Patent Application Publication
(10) Pub. No.: US 2004/0086420 A1
(43) Pub. Date: May 6, 2004

(54) Methods for sterilizing serum or plasma

(76) Inventors: Martin J. MacPhee, North Potomac, MD (US); Dawson Beall, Wheaton, MD (US); William N. Drohan, Springfield, VA (US); Belinda Wilmer, Frederick, MD (US); Imrana Iqbal, Germantown, MD (US); Shirley Miekka, Gaithersburg, MD (US); Halina Malgorzata Zbikowska, Lodz (PL)

Correspondence Address: FLESHNER & KIM, LLP, P.O. BOX 221200, CHANTILLY, VA, 20153, US

(21) Appl. No.: 10/460,654

(22) Filed: Jun. 13, 2003

Related U.S. Application Data
(63) Continuation-in-part of application No. 09/533,547, filed on Mar. 23, 2000.

(51) Int. Cl. A61L 2/08

(52) U.S. Cl. 422/22; 435/2

(57) ABSTRACT

Methods are disclosed for sterilizing serum or plasma to reduce the level of one or more active biological contaminants or pathogens therein, such as viruses, bacteria, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single or multicellular parasites. The methods involve sterilizing serum or plasma with irradiation.

### BACKGROUND OF THE INVENTION

1. Field of the Invention

The present invention relates to methods for sterilizing serum or plasma to reduce the level of one or more active biological contaminants or pathogens therein, such as viruses, bacteria (including inter- and intracellular bacteria, such as mycoplasmas, ureaplasmas, nanobacteria, chlamydia, rickettsias), yeasts, molds, fungi, prions or similar agents responsible, alone or in combination, for transmissible spongiform encephalopathies CTSEs) and/or single or multicellular parasites. The present invention particularly relates to methods of sterilizing serum or plasma with irradiation.

2. Background of the Related Art

Many biological materials that are prepared for human, veterinary, diagnostic and/or experimental use may contain unwanted and potentially dangerous biological contaminants or pathogens, such as viruses, bacteria, in both vegetative and spore states, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single-cell or multicellular parasites. Consequently, it is of utmost importance that any biological contaminant or pathogen in the biological material be inactivated before the product is used. This is especially critical when the material is to be administered directly to a patient, for example in blood transfusions, blood factor replacement therapy, serum or plasma implants, including organ transplants, and other forms of human and/or other animal therapy corrected or treated by surgical implantation, intravenous, intramuscular or other forms of injection or introduction. This is also critical for the various biological materials that are prepared in media or via the culture of cells, or recombinant cells which contain various types of plasma and/or plasma derivatives or other biologic materials and which may be subject to mycoplasmal, prion, ureaplasmal, bacterial, viral and/or other biological contaminants or pathogens.

The importance of ready availability of effective techniques is apparent regardless of the source of the biological material. All living cells and multi-cellular organisms can be infected with viruses and other pathogens. Thus, the products of unicellular natural or recombinant organisms or serum or plasmas virtually always carry a risk of pathogen contamination. In addition to the risk that the producing cells or cell cultures may be infected, the processing of these and other biological materials also creates opportunities for environmental contamination. The risks of infection are more apparent for multicellular natural and recombinant organisms, such as transgenic animals. Interestingly, even products from species as different from humans as transgenic plants carry risks, both due to processing contamination as described above, and from environmental contamination in the growing facilities, which may be contaminated by pathogens from the environment or infected organisms that co-inhabit the facility along with the desired plants. For example, a crop of transgenic corn grown out doors, could be expected to be exposed to rodents such as mice during the growing season. Mice can harbor serious human pathogens such as the frequently fatal Hanta virus. Since these animals would be undetectable in the growing crop, viruses shed by the animals could be carried into the transgenic material at harvest. Indeed, such rodents are notoriously difficult to control, and may gain access to a crop during sowing, growth, harvest or storage. Likewise, contamination from overflying or perching birds has the potential to transmit such serious pathogens as the causative agent for psittacosis. Thus, any biological material, regardless of its source, may harbor serious pathogens that must be removed or inactivated prior to administration of the material to a recipient human or other animal.

In conducting experiments to determine the ability of technologies to inactivate viruses, the actual viruses of concern are seldom utilized. This is a result of safety concerns for the workers conducting the tests, and the difficulty and expense associated with facilities for containment and waste disposal. In their place, model viruses of the same family and class are usually used. In general, it is acknowledged that the most difficult viruses to inactivate are those with an outer shell made up of proteins, and that among these, the most difficult to inactivate ate those of the smallest size. This has been shown to be true for gamma irradiation and most other forms of radiation because these viruses' diminutive size is associated with a small genome. The magnitude of direct effects of radiation upon a molecule is directly proportional to the size of the molecule; that is, the larger the target molecule, the greater is the effect. As a corollary, it has been shown for gamma-irradiation that the smaller the viral genome, the higher is the radiation dose required to inactive it.

Among the viruses of concern for both human and animal-derived biological materials, the smallest, and thus most difficult to inactivate, belong to the family of Parvoviruses and the slightly larger protein-coated Hepatitis virus. In humans, the Parvovirus B19, and Hepatitis A are the agents of concern. In porcine-derived materials, the smallest corresponding virus is Porcine Parvovirus. Since this virus is harmless to humans, it is frequently chosen as a model virus for the human B19 Parvovirus. The demonstration of inactivation of this model parvovirus is considered adequate proof that the method employed will kill human B19 virus and Hepatitis A, and, by extension, that it will also kill the larger and less hardy viruses, such as HIV, CMV, Hepatitis B, Hepatitis C, and others.

More recent efforts have focused on methods to remove or inactivate contaminants in products intended for use in humans and other animals. Such methods include heat treating, filtration and the addition of chemical inactivants or sensitizers to the product.

According to current standards of the U.S. Food and Drug Administration, heat treatment of biological materials may require heating to approximately 60° C. for a minimum of 10 hours, which can be damaging to sensitive biological materials. Indeed, heat inactivation can destroy 50% or more of the biological activity of certain biological materials. Serum or plasmas are particularly sensitive to these high temperature treatments.

Filtration involves filtering the product in order to physically remove contaminants. Unfortunately, this method may also remove products that have a high molecular weight. Further, in certain cases, small viruses may not be removed by the filter.

The procedure of chemical sensitization involves the addition of noxious agents which bind to the DNA/RNA of the virus, and which are activated either by UV or other radiation. This radiation produces reactive intermediates and/or free radicals which bind to the DNA/RNA of the virus, break the chemical bonds in the backbone of the DNA/RNA, and/or cross-link or complex it in such a way that the virus can no longer replicate. This procedure requires that unbound sensitizer be washed from products since the sensitizers are toxic, if not mutagenic or carcinogenic, and cannot be administered to a patient.

Irradiating a product with gamma radiation is another method of sterilizing a product. Gamma radiation is effective in destroying viruses and bacteria when given in high total doses (Keathly, et al., “Is There Life After Irradiation? Part 2,” BioPharm July-August, 1993, and Leitman, “Use of Blood Cell Irradiation in the Prevention of Post Transfusion Graft-vs-Host Disease,” Transfusion Science 10:219-239(1989)). The published literature in this area, however, teaches that gamma radiation can be damaging to radiation sensitive products, such as blood, blood products, protein and protein-containing products. In particular, it has been shown that high radiation doses are injurious to red cells, platelets and granulocytes (Leitman). U.S. Pat. No. 4,620,908 discloses that protein products must be frozen prior to irradiation in order to maintain the viability of the protein product. This patent concludes that “[i] f the gamma irradiation were applied while the protein material was at, for example, ambient temperature, the material would be also completely destroyed, that is the activity of the material would be rendered so low as to be virtually ineffective.” Unfortunately, many sensitive biological materials, such as monoclonal antibodies (Mab), may lose viability and activity if subjected to freezing for irradiation purposes and then thawing prior to administration to a patient.

In view of the difficulties discussed above, there remains a need for methods of sterilizing biological materials that are effective for reducing the level of active biological contaminants or pathogens without an adverse effect on the material(s).

The above references are incorporated by reference herein where appropriate for appropriate teachings of additional or alternative details, features and/or technical background.

### SUMMARY OF THE INVENTION

An object of the invention is to solve at least the above problems and/or disadvantages and to provide at least the advantages described hereinafter.

An object of the invention is to solve at least the related art problems and disadvantages, and to provide at least the advantages described hereinafter.

Accordingly, it is an object of the present invention to provide methods of sterilizing serum or plasma by reducing the level of active biological contaminants or pathogens without adversely affecting the serum or plasma. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof

In accordance with these and other objects, a first embodiment of the present invention is directed to a method for sterilizing serum or plasma that is sensitive to radiation, the method comprising irradiating the serum or plasma with radiation for a time effective to sterilize the serum or plasma at a rate effective to sterilize the serum or plasma and to protect the serum or plasma from the radiation.

Another embodiment of the present invention is directed to a method for sterilizing serum or plasma that are sensitive to radiation, comprising: (i) applying to the serum or plasma at least one stabilizing process selected from the group consisting of: (a) adding to the serum or plasma at least one stabilizer; (b) reducing the residual solvent content of the serum or plasma; (c) reducing the temperature of the serum or plasma; (d) reducing the oxygen content of the serum or plasma; (e) adjusting or maintaining the pH of the serum or plasma; and (f) adding to the serum or plasma at least one non-aqueous solvent; and (ii) irradiating the serum or plasma with a suitable radiation at an effective rate for a time effective to sterilize the serum or plasma, wherein the at least one stabilizing process and the rate of irradiation are together effective to protect the serum or plasma from the radiation.

Another embodiment of the present invention is directed to methods for prophylaxis or treatment of a condition or disease in a mammal comprising introducing into a mammal in need thereof serum or plasma sterilized according to the methods of the present invention.

Another embodiment of the present invention is directed to a composition comprising serum or plasma and at least one stabilizer in an amount effective to preserve the serum or plasma for its intended use following sterilization with radiation.

Another embodiment of the present invention is directed to a composition comprising serum or plasma sterilized according to the methods of the present invention.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1 shows percent recovery of marker proteins after gamma irradiation of plasma at 2.5 kGy/hr to a total dose of 50 kGy according to preferred embodiments of the present invention;

FIG. 2 shows percent recovery of marker proteins after gamma irradiation of plasma containing stabilizers according to preferred embodiments of the present invention;

FIG. 3 shows Rubella Ag-Reactive IgG Recovery Data for plasma treated according to preferred embodiments of the present invention;

FIG. 4 shows the results of SDS-PAGE analysis of plasma treated according to preferred embodiments of the present invention;

FIG. 5 shows oxidized LDL in frozen plasma in the presence or absence of stabilizers;

FIG. 6 shows the turbidity of units of human plasma after irradiation on dry ice;

FIG. 7 shows SDS-PAGE results of albumin gamma irradiated at about 70° C. according to preferred embodiments of the present invention;

FIG. 8 shows Recovery of PD-Factor VIII in the presence or absence of stabilizers according to preferred embodiments of the present invention;

FIG. 9 shows the functional activity of urokinase irradiated with gamma radiation in the presence or absence of ascorbate;

FIG. 10 shows the effect of gamma irradiation on hemoglobin liquid irradiated at about −75° C.;

FIG. 11 shows inactivation of PPV in albumin irradiated according to preferred embodiments of the present invention;

FIG. 12 shows PPV and Sindbis inactivation in frozen human plasma in the presence of absence of stabilizers according to preferred embodiments of the present invention;

FIG. 13 shows prion inactivation in human albumin irradiated according to preferred embodiments of the present invention;

FIG. 14 shows the results of turbidity analysis of irradiated Hyclone FBS irradiated to 40 kGy with gamma irradiation at various temperatures;

FIG. 15 shows the results of turbidity analysis of FBS irradiated to 40 kGy wherein the FBS is complement inactivated prior to and after sterilization according to preferred embodiments of the present invention;

FIG. 16 shows the results of turbidity analysis of FBS irradiated according to preferred embodiments of the present invention in the presence or absence of stabilizers;

FIG. 17 shows the results of turbidity analysis of FBS sterilized according to preferred embodiments of the present invention in the presence or absence of pyruvate;

FIG. 18 shows the results of SDS-PAGE analyis of FBS sterilized according to preferred embodiments of the present invention in the presence or absence of stabilizers;

FIG. 19 shows the results of SDS-PAGE analysis of FBS sterilized according to preferred embodiments of the present invention in the presence or absence of various stabilizers;

FIG. 20 shows the thermal stability of FBS sterilized according to preferred embodiments of the present invention;

FIG. 21 shows the thermal stability of FBS sterilized according to preferred embodiments of the present invention in the presence or absence of pyruvate;

FIG. 22 shows the relationship between total dose and temperature of FBS sterilized according to preferred embodiments of the present invention;

FIG. 23 shows the relationship between total dose and temperature of FBS sterilized according to preferred embodiments of the present invention in the presence or absence of stabilizers;

FIG. 24 shows the results of SDS-PAGE analysis of FBS sterilized according to preferred embodiments of the present invention at various temperatures;

FIG. 25 shows the results of SDS-PAGE analysis of FBS sterilized according to preferred embodiments of the present invention at various temperatures;

FIG. 26 shows the results of SDS-PAGE analysis of FBS sterilized according to preferred embodiments of the present invention in the presence or absence of stabilizers at various temperatures;

FIG. 27 shows results of SDS-PAGE analysis of FBS sterilized according to preferred embodiments of the present invention in the presence of pyruvate and at various temperature;

FIG. 28 shows the results of SDS-PAGE analysis of FBS irradiated to a total dose of about 40 kGy according to preferred embodiments of the present invention at various temperatures;

FIG. 29 shows the results of SDS-PAGE analysis of FBS irradiated to a total dose of about 40 kGy according to preferred embodiments of the present invention in the presence of pyruvate; and

FIG. 30 shows CHO-IK1 cell growth in fetal calf serum sterilized according to preferred embodiments of the present invention in the presence or absence of stabilizers.

### DETAILED DESCRIPTION OF PREFERRED EMBODIMENTS

A. Definitions

Unless defined otherwise, all technical and scientific terms used herein are intended to have the same meaning as is commonly understood by one of ordinary skill in the relevant art.

As used herein, the singular forms “a,” “an,” and “the” include the plural reference unless the context clearly dictates-otherwise.

As used herein, the term “sterilize” is intended to mean a reduction in the level of at least one active biological contaminant or pathogen found in the serum or plasma being treated according to the present invention.

As used herein, the term “non-aqueous solvent” is intended to mean any liquid other than water in which a biological material, such as serum or plasma, may be dissolved or suspended or which may be disposed within a biological material, such as serum or plasma, and includes both inorganic solvents and, more preferably, organic solvents. Illustrative examples of suitable non-aqueous solvents include, but are not limited to, the following: alkanes and cycloalkanes, such as pentane, 2-methylbutane (isopentane), heptane, hexane, cyclopentane and cyclohexane; alcohols, such as methanol, ethanol, 2-methoxyethanol, isopropanol, n-butanol, t-butyl alcohol, and octanol; esters, such as ethyl acetate, 2-methoxyethyl acetate, butyl acetate and benzyl benzoate; aromatics, such as benzene, toluene, pyridine, xylene; ethers, such as diethyl ether, 2-ethoxyethyl ether, ethylene glycol dimethyl ether and methyl t-butyl ether; aldehydes, such as formaldehyde and glutaraldehyde; ketones, such as acetone and 3-pentanone (diethyl ketone); glycols, including both monomeric glycols, such as ethylene glycol and propylene glycol, and polymeric glycols, such as polyethylene glycol (PEG) and polypropylene glycol (PPG), e.g., PPG 400, PPG 1200 and PPG 2000; acids and acid anhydrides, such as formic acid, acetic acid, trifluoroacetic acid, phosphoric acid and acetic anhydride; oils, such as cottonseed oil, peanut oil, culture media, polyethylene glycol, poppyseed oil, safflower oil, sesame oil, soybean oil and vegetable oil; amines and armides, such as piperidine, N,N-dimethylacetamide and N,Ndeimethylformamide; dimethylsulfoxide (DMSO); nitriles, such as benzonitrile and acetonitrile; hydrazine; detergents, such as polyoxyethylenesorbitan monolaurate (fween 20) and monooleate (Tween 80), Triton and sodium dodecyl sulfate; carbon disulfide; halogenated solvents, such as dichloromethane, chloroform, carbon tetrachloride, 1,2dichlorobenzene, 1,2-dichloroethane, tetrachloroethylene and 1-chlorobutane; furans, such as tetrahydrofuran; oxanes, such as 1,4-dioxane; and glycerin/glycerol. Particularly preferred examples of suitable non-aqueous solvents include non-aqueous solvents which also function as stabilizers, such as ethanol and acetone.

As used herein, the term “biological contaminant or pathogen” is intended to mean a biological contaminant or pathogen that, upon direct or indirect contact with a biological material, such as serum or plasma, may have a deleterious effect on the biological material or upon a recipient thereof. Such other biological contaminants or pathogens include the various viruses, bacteria, in both vegetative and spore states, (including inter- and intracellular bacteria, such as mycoplasmas, ureaplasmas, nanobacteria, chlamydia, rickettsias), yeasts, molds, fungi, prions or similar agents responsible, alone or in combination, for TSEs and/or single or multicellular parasites known to those of skill in the art to generally be found in or infect biological materials. Examples of other biological contaminants or pathogens include, but are not limited to, the following: viruses, such as human immunodeficiency viruses and other retroviruses, herpes viruses, filoviruses, circoviruses, paramyxovrruses, cytomegaloviruses, hepatitis viruses (including hepatitis A, B. C, and D variants thereof, among others), pox viruses, toga viruses, Ebstein-Barr viruses and parvoviruses; bacteria, such as Escherichia, Bacillus, Campylobacter, Streptococcus and Staphylococcus; nanobacteria; parasites, such as Trypanosoma and malarial parasites, including Plasmodium species; yeasts; molds; fungi; mycoplasmas and ureaplasmas; chlamydia; rickettsias, such as Coxiella burnetti; and prions and similar agents responsible, alone or in combination, for one or more of the disease states known as transmissible spongiform encephalopathies (TSEs) in mammals, such as scrapie, transmissible mink encephalopathy, chronic wasting disease (generally observed in mule deer and elk), feline spongiform encephalopathy, bovine spongiform encephalopathy (mad cow disease), Creutzfeld-Jakob disease (including variant CJD), Fatal Familial Insomnia, Gerstmann-Straeussler-Scheinker syndrome, kuru and Alpers syndrome. As used herein, the term “active biological contaminant or pathogen” is intended to mean a biological contaminant or pathogen that is capable of causing a deleterious effect, either alone or in combination with another factor, such as a second biological contaminant or pathogen or a native protein (wild-type or mutant) or antibody, in a biological material, such as serum or plasma, and/or a recipient thereof.

As used herein, the term “a biologically compatible solution” is intended to mean a solution to which a biological material, such as serum or plasma, may be exposed, such as by being suspended or dissolved therein, and retain its essential biological and physiological characteristics. Such solutions may be of any suitable pH, tonicity, concentration and/or ionic strength.

As used herein, the term “a biologically compatible buffered solution” is intended to mean a biologically compatible solution having a pH and osmotic properties (e.g., tonicity, osmolality and/or oncotic pressure) suitable for maintaining the integrity of the material(s) therein, such as serum or plasma. Suitable biologically compatible buffered solutions typically have a pH between 2 and 8.5 and are isotonic or only moderately hypotonic or hypertonic. Biologically compatible buffered solutions are known and readily available to those of skill in the art. Greater or lesser pH and/or tonicity may also be used in certain applications. The ionic strength of the solution may be high or low, but is typically similar to the environments in which the serum or plasma is intended to be used.

As used herein, the term “stabilizer” is intended to mean a compound or material that, alone and/or in combination, reduces damage to the biological material being irradiated to a level that is insufficient to preclude the safe and effective use of the material. Illustrative examples of stabilizers that are suitable for use include, but are not limited to, the following, including structural analogs and derivatives thereof: antioxidants; free radical scavengers, including spin traps, such as tert-butyl-nitrosobutane (tNB), a-phenyl-tert-butylnitrone (PBN), 5,5-dimethylpyrroline-N-oxide (DMPO), tert-butylnitrosobenzene (BNB), a-(4-pyridyl-1-oxide)-N-tert-butylnitrone (4-POBN) and 3,5-dibromo-4-nitroso-benzenesulphonic acid PBNBS); combination stabilizers, i.e., stabilizers which are effective at quenching both Type I and Type II photodynamic reactions; and ligands, ligand analogs, substrates, substrate analogs, modulators, modulator analogs, stereoisomers, inhibitors, and inhibitor analogs, such as heparin, that stabilize the molecule(s) to which they bind. Preferred examples of additional stabilizers include, but are not limited to, the following: fatty acids, including 6,8-dimercapto-octanoic acid (lipoic acid) and its derivatives and analogues (alpha, beta, dihydro, bisno and tetranor lipoic acid), thioctic acid, 6,8-dimercapto-octanoic acid, dihydrolopoate PL-6,8-dithioloctanoic acid methyl ester), lipoamide, bisonor methyl ester and tetranor-dihydrolipoic acid, omega-3 fatty acids, omega-6 fatty acids, omega-9 fatty acids, furan fatty acids, oleic, linoleic, linolenic, arachidonic, eicosapentaenoic (EPA), docosahexaenoic (DHA), and palmitic acids and their salts and derivatives; carotenes, including alpha-, beta-, and gamma-carotenes; Co-Q10; xanthophylls; sucrose, polyhydric alcohols, such as glycerol, mannitol, inositol, and sorbitol; sugars, including derivatives and stereoisomers thereof, such as xylose, glucose, ribose, mannose, fructose, erythrose, threose, idose, arabinose, lyxose, galactose, allose, altrose, gulose, talose, and trehalose; amino acids and derivatives thereof, including both D- and L-forms and mixtures thereof, such as arginine, lysine, alanine, valine, leucine, isoleucine, proline, phenylalanine, glycine, serine, threonine, tyrosine, asparagine, glutamine, aspartic acid, histidine, N-acetylcysteine WAC), glutamic acid, tryptophan, sodium capryl N-acetyl tryptophan, and methionine; azides, such as sodium azide; enzymes, such as Superoxide Dismutase (SOD), Catalase, and Δ4, Δ5 and Δ6 desaturases; uric acid and its derivatives, such as 1,3-dimethyluric acid and dimethylthiourea; allopurinol; thiols, such as glutathione and reduced glutathione and cysteine; trace elements, such as selenium, chromium, and boron; vitamins, including their precursors and derivatives, such as vitamin A, vitamin C (including its derivatives and salts such as sodium ascorbate and palmitoyl ascorbic acid) and vitamin E (and its derivatives and salts such as alpha-, beta-, gamma-, delta-, epsilon-, zeta-, and eta-tocopherols, tocopherol acetate and alpha-tocotrienol); chromanol-alpha-C6; 6-hydroxy-2,5,7,8-tetramethylchroma-2 carboxylic acid Trolox) and derivatives; extraneous proteins, such as gelatin and albumin; tris-3-methyl-1-phenyl-2-pyrazolin-5-one MCI-186); citiolone; puercetin; chrysin; dimethyl sulfoxide (MSO); piperazine diethanesulfonic acid (PIPES); imidazole; methoxypsoralen (MOPS); 1,2-dithiane-4,5-diol; reducing substances, such as butylated hydcroxyanisole (BHA) and butylated hydroxytoluene (BHT); cholesterol, including derivatives and its various oxidized and reduced forms thereof, such as low density lipoprotein (LDL), high density lipoprotein (HDL), and very low density lipoprotein (VLDL); probucol; indole derivatives; thimerosal; lazaroid and tirilazad mesylate; proanthenols; proanthocyanidins; ammonium sulfate; Pegorgotein (PEG-SOD); N-tert-butyl-alpha-phenylnitrone (PBN); 4-hydroxy-2,2,6,6-tetramethylpiperidin-1-oxyl (Tempol); mixtures of ascorbate, urate and Trolox C (Asc/urate/Trolox C); proteins, such as albumin, and peptides of two or more amino acids, any of which may be either naturally occurring amino acids, i.e., L-amino acids, or non-naturally occurring amino acids, i.e., D-amino acids, and mixtures, derivatives, and analogs thereof, including, but not limited to, arginine, lysine, alanine, valine, leucine, isoleucine, proline, phenylalanine, glycine, histidine, glutamic acid, tryptophan (Trp), serine, threonine, tyrosine, asparagine, glutamine, aspartic acid, cysteine, methionine, and derivatives thereof, such as N-acetylcysteine (NAC) and sodium capryl N-acetyl tryptophan, as well as homologous dipeptide stabilizers (composed of two identical amino acids), including such naturally occurring amino acids, as Gly-Gly (glycylglycine) and Trp-Trp, and heterologous dipeptide stabilizers (composed of different amino acids), such as carnosine (β-alanyl-histidine), anserine (β-alanyl-methylhistidine), and Gly-Trp; and flavonoids/flavonols, such as diosmin, quercetin, rutin, silybin, silidianin, silicristin, silymarin, apigenin, apiin, chrysin, morin, isoflavone, flavoxate, gossypetin, myricetin, biacalein, kaempferol, curcumin, proanthocyanidinr B2-3-O-gallate, epicatechin gallate, epigallocatechin gallate, epigallocatechin, gallic acid, epicatechin, dihydroquercetin, quercetin chalcone, 4,4′-dihydroxy-chalcone, isoliqutiritigenin, phloretin, coumestrol, 4′,7-dihydroxy-flavanone, 4′,5-dihydroxy-flavone, 4′,6-dihydroxy-flavone, luteolin, galangin, equol, biochanin A, daidzein, formononetin, genistein, amentoflavone, bilobetin, taxifolin, delphinidin, malvidin, petunidin, pelargonidin, malonylapiin, pinosylvin, 3-methoxyapigenin, leucodelphinidin, dihydrokaempferol, apigenin 7-O-glucoside, pycnogenol, aminoflavone, purpurogallin fisetin, 2′,3′-dihydroxyflavone, 3-hydroxyflavone, 3′,4′-dihydtoxyflavone, catechin, 7-flavonoxyacetic acid ethyl ester, catechin, hesperidin, and naringin. Particularly preferred examples include single stabilizers or combinations of stabilizers that are effective at quenching both Type I and Type II photodynanic reactions, and volatile stabilizers, which can be applied as a gas and/or easily removed by evaporation, low pressure, and similar methods. Additional preferred examples for use in the methods of the present invention include hydrophobic stabilizers. Other preferred stabilizers according to the methods of the present invention include compounds capable of preventing the generation of free radicals during irradiation. Other preferred stabilizers according to the methods of the present invention include compounds capable of preventing the generation of reactive oxygen species during irradiation. Still other preferred stabilizers according to the present invention include α-keto acids, including but not limited to lactate and pyruvate.

As used herein, the term “residual solvent content” is intended to mean the amount or proportion of freely-available liquid in the biological material. Freely-available liquid means the liquid, such as water and/or an organic solvent (e.g., ethanol, isopropanol, polyethylene glycol, etc.), present in the biological material being sterilized that is not bound to or complexed with one or more of the non-liquid components of the biological material. Freely-available liquid includes intracellular water and/or other solvents. The residual solvent contents related as water referenced herein refer to levels determined by the FDA approved, modified Karl Fischer method (Meyer and Boyd, Analytical Chem., 31:215-219, 1959; May, et al., J. Biol. Standardization, 10:249-259, 1982; Centers for Biologics Evaluation and Research, FDA, Docket No. 89D-0140, 83-93; 1990) or by near infrared spectroscopy. Quantitation of the residual levels of water or other solvents may be determined by means well known in the art, depending upon which solvent is employed. The proportion of residual solvent to solute may also be considered to be a reflection of the concentration of the solute within the solvent. When so expressed, the greater the concentration of the solute, the lower the amount of residual solvent.

As used herein, the term “sensitizer” is intended to mean a substance that selectively targets viruses, bacteria, in both vegetative and spore states, (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs, rendering them more sensitive to inactivation by radiation, therefore permitting the use of a lower rate or dose of radiation and/or a shorter time of irradiation than in the absence of the sensitizer. Illustrative examples of suitable sensitizers include, but are not limited to, the following: psoralen and its derivatives and analogs (including 3-carboethoxy psoralens); inactines and their derivatives and analogs; angelicins, khellins and coumarins which contain a halogen substituent and a water solubilization moiety, such as quaternary ammonium ion or phosphonium ion; nucleic acid binding compounds; brominated hematoporphyrin; phthalocyanines; purpurins; porphyrins; halogenated or metal atom-substituted derivatives of dihematoporphyrin esters, hematoporphyrin derivatives, benzoporphyrin derivatives, hydrodibenzoporphyrin dimaleimade, hydrodibenzoporphyrin, dicyano disulfone, tetracarbethoxy hydrodibenzoporphyrin, and tetracarbethoxy hydrodibenzoporphyrin dipropionamide; doxotubicin and daunomycin, which may be modified with halogens or metal atoms; netropsin; BD peptide, S2 peptide; S-303 (ALE compound); dyes, such as hypericin, methylene blue, eosin, fluoresceins (and their derivatives), flavins, merocyanine 540; photoactive compounds, such as bergapten; and SE peptide. In addition, atoms which bind to prions, and thereby increase their sensitivity to inactivation by radiation, may also be used. An illustrative example of such an atom would be the Copper ion, which binds to the prion protein and, with a Z number higher than the other atoms in the protein, increases the probability that the prion protein will absorb energy during irradiation, particularly gamma irradiation.

As used herein, the term “radiation” is intended to mean radiation of sufficient energy to sterilize at least some component of the irradiated biological material. Types of radiation include, but are not limited to, the following: (i) corpuscular (streams of subatomic particles such as neutrons, electrons, and/or protons); (ii) electromagnetic (originating in a varying electromagnetic field, such as radio waves, visible (both mono and polychromatic) and invisible light, infrared, ultraviolet radiation, x-radiation, and gamma rays and mixtures thereof); and (iii) sound and pressure waves. Such radiation is often described as either ionizing (capable of producing ions in irradiated materials) radiation, such as gamma rays, and non-ionizing radiation, such as visible light. The sources of such radiation may vary and, in general, the selection of a specific source of radiation is not critical provided that sufficient radiation is given in an appropriate time and at an appropriate rate to effect sterilization. In practice, gamma radiation is usually produced by isotopes of Cobalt or Cesium, while UV and X-rays are produced by machines that emit UV and X-radiation, respectively, and electrons are often used to sterilize materials in a method known as “Ebeam” irradiation that involves their production via a machine. Visible light, both mono- and polychromatic, is produced by machines and may, in practice, be combined with invisible light, such as infrared and UV, that is produced by the same machine or a different machine.

As used herein, the term “serum or plasma” is intended to mean the fluid component of blood derived or obtained from a living organism (in dried or liquid form), prenatal, postnatal, itnature, mature or adult. The serum may contain at least one clotting factor (in dried or liquid form) or component involved in clotting (in dried or liquid form). The term “clotting factor(s) or component(s) involved in clotting” is intended to include any blood component involved in clotting and includes, but is not limited to, Factor I, Factor II, Factor III, Factor V, Factor VI, Factor VII, Factor VIII, Factor IX, Factor X, Factor XI, Factor XII, Factor XIII, von Willebrands factor, Factor Ia, Factor Ia, Factor Ila, Factor Va, Factor VIa, Factor VIIa, Factor VIIIa, Factor IXa, Factor Xa, Factor Xla, Factor XIIa and Factor Xiiia, as well as components such as Protein C, Protein S, Serum or plasma Factor Pathway-Inhibitor (TFPI), Serum or plasma Factor (serum or plasma thromboplastin), procoagulant phospholipid, and thrombomodulin, all of which may be obtained or derived from any animal, including genetic, as well as chemically and biotechnically produced, variants thereof. Examples of serum include, but are not limited to, bovine serum (such as fetal bovine serum and fetal calf serum), ovine serum, equine serum, caprine serum, human serum and porcine serum. The serum or plasma according to the methods of the present invention may contain additives known in the art. Moreover, the serum or plasma may be treated by methods known in the art, for instance by heating to reduce complement activity.

As used herein, the term “to protect” is intended to mean to reduce any damage to the biological material, such as serum or plasma, being irradiated, that would otherwise result from the irradiation of that material, to a level that is insufficient to preclude the safe and effective use of the material following irradiation. In other words, a substance or process “protects” a biological material, such as serum or plasma, from radiation if the presence of that substance or carrying out that process results in less damage to the material from irradiation than in the absence of that substance or process. Thus, a biological material, such as serum or plasma, may be used safely and effectively after irradiation in the presence of a substance or following performance of a process that “protects” the material, but could not be used with as great a degree of safety or as effectively after irradiation under identical conditions but in the absence of that substance or the performance of that process.

As used herein, an “acceptable level” of damage may vary depending upon certain features of the particular method(s) of the present invention being employed, such as the nature and characteristics of the particular serum or plasma and/or non-aqueous solvent(s) being used, and/or the intended use of the material being irradiated, and can be determined empirically by one skilled in the art. An “unacceptable level” of damage would therefore be a level of damage that would preclude the safe and effective use of the biological material, such as serum or plasma, being sterilized. The particular level of damage in a given biological material may be determined using any of the methods and techniques known to one skilled in the art.

B. Particularly Preferred Embodiments

A first preferred embodiment of the present invention is directed to a method for sterilizing serum or plasma that is sensitive to radiation, the method comprising irradiating the serum or plasma with radiation for a time effective to sterilize the serum or plasma at a rate effective to sterilize the serum or plasma and to protect the serum or plasma from the radiation.

A second preferred embodiment of the present invention is directed to a method for sterilizing serum or plasma that are sensitive to radiation, comprising: (i) applying to the serum or plasma at least one stabilizing process selected from the group consisting of: (a) adding to the serum or plasma at least one stabilizer; (b) reducing the residual solvent content of the serum or plasma; (c) reducing the temperature of the serum or plasma; (d) reducing the oxygen content of the serum or plasma; (e) adjusting or maintaining the pH of the serum or plasma; and (f) adding to the serum or plasma at least one non-aqueous solvent; and (ii) irradiating the serum or plasma with a suitable radiation at an effective rate for a time effective to sterilize the serum or plasma, wherein the at least one stabilizing process and the rate of irradiation are together effective to protect the serum or plasma from the radiation.

Another third preferred embodiment of the present invention is directed to a composition comprising serum or plasma and at least one stabilizer in an amount effective to preserve the serum or plasma for its intended use following sterilization with radiation.

Another fourth preferred embodiment of the present invention is directed to a. Another embodiment of the present invention is directed to a composition comprising serum or plasma sterilized according to the methods of the present invention.

Another preferred embodiment of the present invention is directed to a composition comprising serum or plasma and at least one stabilizer in an amount effective to preserve the serum or plasma for their intended use following sterilization with radiation.

Another preferred embodiment of the present invention is directed to a composition comprising serum or plasma, at least one non-aqueous solvent and/or at least one stabilizer in an amount effective to preserve the serum or plasma for their intended use following sterilization with radiation.

The non-aqueous solvent is preferably a non-aqueous solvent that is not prone to the formation of free-radicals upon irradiation, and more preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

According to certain embodiments of the present invention, the serum or plasma may contain a mixture of water and a non-aqueous solvent, such as ethanol and/or acetone. In such embodiments, the non-aqueous solvent(s) is (are) preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are also stabilizers, such as ethanol and acetone.

According to certain methods of the present invention, at least one stabilizer is added prior to irradiation of the serum or plasma. The at least one stabilizer is preferably added to the serum or plasma in an amount that is effective to protect the serum or plasma from the radiation. Alternatively, the stabilizer is added to the serum or plasma in an amount that, together with a non-aqueous solvent or the effective rate, is effective to protect the serum or plasma from the radiation. Suitable amounts of stabilizer may vary depending upon certain features of the particular method(s) of the present invention being employed, such as the particular stabilizer being used and/or the nature and characteristics of the particular serum or plasma being irradiated and/or its intended use, and can be determined empirically by one skilled in the art.

According to certain preferred embodiments of the present invention, the serum or plasma contains at least one stabilizer in a concentration of at least 1 nM, at least 1 mM, at least 2 mM, at least 5 mM, at least 10 mM, at least 25 mM at least 50 MM or at least 100 mM. Preferred ranges of stabilizer concentrations include, but are not limited to, from 0.1 mM to 10 mM, from 0.1 to 50 mM, from 10 to 100 mM, from 10 mM to 50 mM and from 50 to 100 mM.

In certain preferred embodiments of the present invention, the stabilizer is an amino acid, such as histidine, a vitamin, for instance ascorbate, or an α-keto acid. Such stabilizers may be used alone of in combination. Suitable a-keto acids include, but are not limited to, lactate and pyruvate. Preferably, the serum or plasma includes pyruvate or a mixture of histidine and ascorbate.

According to a preferred embodiment of the present invention, the serum or plasma contains pyruvate, histidine, ascorbate or combinations thereof. Preferably, the serum or plasma contains pyruvate in a concentration of at least 50 mM. The concentration of histidine is preferably at least 50 mM and more preferably at least 100 mM. Ascorbate is preferably present in a concentration of at least 0.1 mM, more preferably at least 10 mM and most preferably at least 100 mM.

In other preferred embodiments of the present, the serum or plasma contains histidine and ascorbate. According to such embodiments, the serum or plasma preferably contains histidine in a concentration of at least 50 mM and ascorbate in a concentration of at least 10 mM, more preferably 100 mM.

According to certain methods of the present invention, the residual solvent content of the serum or plasma is reduced prior to irradiation of the serum or plasma with radiation. The residual solvent content is preferably reduced to a level that is effective to protect the serum or plasma from the radiation. Suitable levels of residual solvent content may vary depending upon certain features of the particular method(s) of the present invention being employed, such as the nature and characteristics of the particular serum or plasma being irradiated and/or its intended use, and can be determined empirically by one skilled in the art. There may be serum or plasma for which it is desirable to maintain the residual solvent content to within a particular range, rather than a specific value.

According to certain embodiments of the present invention, when the serum or plasma also contain water, the residual solvent (water) content of serum or plasma may be reduced by dissolving or suspending the serum or plasma in a non-aqueous solvent that is capable of dissolving water. Preferably, such a non-aqueous solvent is not prone to the formation of free-radicals upon irradiation and has little or no dissolved oxygen or other gas(es) that is (are) prone to the formation of free-radicals upon irradiation.

While not wishing to be bound by any theory of operability, it is believed that the reduction in residual solvent content reduces the degrees of freedom of the serum or plasma, reduces the number of targets for free radical generation and may restrict the diffusability of these free radicals and/or reactive oxygen species. Similar results might therefore be achieved by lowering the temperature of the serum or plasma below its eutectic point(s) or below its freezing point(s), or by vitrification to likewise reduce the degrees of freedom of the serum or plasma. These results may permit the use of a higher rate and/or dose of radiation than might otherwise be acceptable. Thus, the methods described herein may be performed at any temperature that doesn't result in unacceptable damage to the serum or plasma, i.e., damage that would preclude the safe and effective use of the serum or plasma. Preferably, the methods described herein are performed at ambient temperature or below ambient temperature, such as below the eutectic point(s) or freezing point(s) of the serum or plasma being irradiated.

In certain embodiments of the present invention, the desired residual solvent content of a particular serum or plasma may be found to lie within a range, rather than at a specific point. Such a range for the preferred residual solvent content of a particular serum or plasma may be determined empirically by one skilled in the art.

The residual solvent content of the serum or plasma may be reduced by any of the methods and techniques known to those skilled in the art for reducing solvent from serum or plasma without producing an unacceptable level of damage to the serum or plasma. Such methods include, but are not limited to, lyophilization, drying, concentration, addition of alternative solvents, evaporation, chemical extraction and vitrification.

A particularly preferred method for reducing the residual solvent content of serum or plasma is lyophilization.

According to certain methods of the present invention, the serum or plasma to be sterilized may be immobilized upon or attached to a solid surface by any means known and available to one skilled in the art. For example, the serum or plasma to be sterilized may be attached to a biological or non-biological substrate.

The radiation employed in the methods of the present invention may be any radiation effective for the sterilization of the serum or plasma being treated. The radiation may be corpuscular, including E-beam radiation. Preferably the radiation is electromagnetic radiation, including x-rays, infrared, visible light, UV light and mixtures of various wavelengths of electromagnetic radiation. A particularly preferred form of radiation is gamma radiation.

According to the methods of the present invention, the serum or plasma is irradiated with the radiation at a rate effective for the sterilization of the serum or plasma, while not producing an unacceptable level of damage to the serum or plasma. Suitable rates of irradiation may vary depending upon certain features of the methods of the present invention being employed, such as the nature and characteristics of the particular serum or plasma, which may contain a non-aqueous solvent, being irradiated; the particular form of radiation involved, and/or the particular biological contaminants or pathogens being inactivated. Suitable rates of irradiation can be determined empirically by one skilled in the art. Preferably, the rate of irradiation is constant for the duration of the sterilization procedure. When this is impractical or otherwise not desired, a variable or discontinuous irradiation may be utilized.

According to the methods of the present invention, the rate of irradiation may be optimized to produce the most advantageous combination of product recovery and time required to complete the operation. Both low (<3 kGy/hour) and high (>3 kGy/hour) rates may be utilized in the methods described herein to achieve such results. The rate of irradiation is preferably selected to optimize the recovery of the serum or plasma while still sterilizing the serum or plasma. Although reducing the rate of irradiation may serve to decrease damage to the serum or plasma, it will also result in longer irradiation times being required to achieve a particular desired total dose. A higher dose rate may therefore be preferred in certain circumstances, such as to mize logistical issues and costs, and may be possible particularly when used in accordance with the methods described herein for protecting serum or plasma from irradiation.

According to a particularly preferred embodiment of the present invention, the rate of irradiation is not more than about 3.0 kGy/hour, more preferably between about 0.1 kGy/hr and 3.0 kGy/hr, even more preferably between about 0.25 kGy/hr and 2.0 kGy/hour, still even more preferably between about 0.5 kGy/hr and 1.5 kGy/hr and most preferably between about 0.5 kGy/hr and 1.0 kGy/hr. In other preferred embodiments of the present invention, the rate of irradiation is not more than about 2.5 kGy/hr, more preferably not more than 2.0 kGy/hr, even more preferably not more than 1.0 kGy/hr and most preferably not more than 0.3 kGy/hr.

According to another particularly preferred embodiment of the present invention, the rate of irradiation is more than 3.0 kGy/hr, more preferably at least 5 kGy/hr, even more preferably at least about 18 kGy/hr, even more preferably at least about 30 kGy/hr and most preferably at least about 45 kGy/hr or greater.

According to the methods of the present invention, the serum or plasma to be sterilized is irradiated with the radiation for a time effective for the sterilization of the serum or plasma. Combined with irradiation rate, the appropriate irradiation time results in the appropriate dose of irradiation being applied to the serum or plasma. Suitable irradiation times may vary depending upon the particular form and rate of radiation involved and/or the nature and characteristics of the particular serum or plasma being irradiated. Suitable irradiation times can be determined empirically by one skilled in the art.

According to the methods of the present invention, the serum or plasma to be sterilized is irradiated with radiation up to a total dose effective for the sterilization of the serum or plasma, while not producing an unacceptable level of damage to the serum or plasma. Suitable total doses of radiation may vary depending upon certain features of the methods of the present invention being employed, such as the nature and characteristics of the particular serum or plasma being irradiated, the particular form of radiation involved, and/or the particular biological contaminants or pathogens being inactivated. Suitable total doses of radiation can be determined empirically by one skilled in the art. Preferably, the total dose of radiation is at least 10 kGy, more preferably at least 25 kGy, even more preferably at least 30 kGy, and still more preferably at least 40 kGy or greater, such as 50 kGy, 55 kGy, 65 kGy, 70 kGy 80 kGy, 90 kGy, 105 kGy, 120 kGy or greater.

The particular geometry of the serum or plasma being irradiated, such as the container holding the serum or plasma, may be determined empirically by one skilled in the art. A preferred embodiment is a geometry that provides for an even rate of irradiation throughout the preparation of serum or plasma. A particularly preferred embodiment is a geometry that results in a short path length for the radiation through the preparation, thus minimizing the differences in radiation dose between the front and back of the preparation. This may be further minimized in some preferred geometries, particularly those wherein the preparation of serum or plasma has a relatively constant radius about its axis that is perpendicular to the radiation source and by the utilization of a means of rotating the preparation of serum or plasma about said axis.

Similarly, according to certain methods of the present invention, an effective package for containing the preparation of serum or plasma during irradiation is one which combines stability under the influence of irradiation, and which minimizes the interactions between the package of serum or plasma and the radiation. Preferred packages maintain a seal against the external environment before, during and post-irradiation, and are not reactive with the preparation of serum or plasma within, nor do they produce chemicals that may interact with the preparation of serum or plasma within. Particularly preferred examples include but are not limited to containers that comprise glasses stable when irradiated, stoppered with stoppers made of tubber or other suitable materials that is relatively stable during radiation and liberates a minimal amount of compounds from within, and sealed with metal crimp seals of aluminum or other suitable materials with relatively low Z numbers. Suitable materials can be determined by measuring their physical performance, and the amount and type of reactive leachable compounds post-irradiation, and by examining other characteristics known to be important to the containment of such biological materials as serum or plasma empirically by one skilled in the art.

According to certain methods of the present invention, an effective amount of at least one sensitizing compound may optionally be added to the serum or plasma prior to irradiation, for example to enhance the effect of the irradiation on the biological contaminant(s) or pathogen(s) therein, while employing the methods described herein to minimize the deleterious effects of irradiation upon the serum or plasma. Suitable sensitizers are known to those skilled in the art, and include psoralens and their derivatives and inactines and their derivatives.

According to the methods of the present invention, the irradiation of the serum or plasma may occur at any temperature that is not deleterious to the serum or plasma being sterilized. According to one preferred embodiment, the serum or plasma being irradiated is at ambient temperature for at least a portion of the irradiation and preferably at the initiation of irradiation. According to an alternate preferred embodiment, the serum or plasma being irradiated is at reduced temperature, i.e., a temperature below ambient temperature, such as at least 4° C., at least 0° C., at least −10° C., at least −20° C., at least −30° C., at least −40° C., at least −50° C., at least −55° C., at least −60° C., at least −70° C., at least −72° C., at least dry ice temperature or at least −196° C., for at least a portion of the irradiation and preferably at the initiation of irradiation. According to this embodiment of the present invention, the serum or plasma being irradiated is preferably at or below the freezing or eutectic point(s) of the serum or plasma and/or the residual solvent therein, for at least a portion of the irradiation and preferably at the initiation of irradiation. Particularly preferred temperature ranges according to certain preferred embodiments of the present invention include, but are not limited to, from −30° C. to −50° C., from −30° C. to −70° C., from −10° C. to −30° C., from −10° C. to −50° C. and from −10° C. to −55° C.

According to another alternate preferred embodiment, the serum or plasma being irradiated is at elevated temperature, i.e., a temperature above ambient temperature, such as 37° C., 60° C., 72° C. or 80° C., for at least a portion of the irradiation and preferably at the initiation of irradiation. While not wishing to be bound by any theory, the use of elevated temperature may enhance the effect of irradiation on the biological contaminant(s) or pathogen(s) and therefore allow the use of a lower total dose of radiation.

Most preferably, the irradiation of the serum or plasma occurs with serum or plasma at a temperature that protects the preparation of serum or plasma from radiation for at least a portion of said irradiation and preferably at the initiation of irradiation. Suitable temperatures can be determined empirically by one skilled in the art.

In certain embodiments of the present invention, the temperature of serum or plasma at which irradiation is performed may be found to lie within a range, rather than at a specific point. Such a range for the preferred temperature may be determined empirically by one skilled in the art.

According to the methods of the present invention, the irradiation of the serum or plasma may occur at any pressure which is not deleterious to the serum or plasma being sterilized. According to one preferred embodiment, the serum or plasma is irradiated at elevated pressure. More preferably, the serum or plasma is irradiated at elevated pressure due to the application of sound waves or the use of a volatile. While not wishing to be bound by any theory, the use of elevated pressure may enhance the effect of irradiation on the biological contaminant(s) or pathogen(s) and/or enhance the protection afforded by one or more stabilizers, and therefore allow the use of a lower total dose of radiation. Suitable pressures can be determined empirically by one skilled in the art.

Generally, according to the methods of the present invention, the pH of the serum or plasma undergoing sterilization is about 7. In some embodiments of the present invention, however, the serum or plasma may have a pH of less than 7, preferably less than or equal to 6, more preferably less than or equal to 5, even more preferably less than or equal to 4, and most preferably less than or equal to 3. In alternative embodiments of the present invention, the serum or plasma may have a pH of greater than 7, preferably greater than or equal to 8, more preferably greater than or equal to 9, even more preferably greater than or equal to 10, and most preferably greater than or equal to 11. According to certain embodiments of the present invention, the pH of the preparation of serum or plasma undergoing sterilization is at or near the isoelectric point of one of the components of the serum or plasma. According to certain preferred embodiments of the present invention, the serum or plasma preferably has a pH of about 6.8 to about 8.1, more preferably about 6.9 to about 7.4 and most preferably about 4.8 to about 5.5. Suitable pH levels can be determined empirically by one skilled in the art.

Similarly, according to the methods of the present invention, the irradiation of the serum or plasma may occur under any atmosphere that is not deleterious to the serum or plasma being treated. According to one preferred embodiment, the serum or plasma is held in a low oxygen atmosphere or an inert atmosphere. When an inert atmosphere is employed, the atmosphere is preferably composed of a noble gas, such as helium or argon, more preferably a higher molecular weight noble gas, and most preferably argon. According to another preferred embodiment, the serum or plasma is held under vacuum while being irradiated. According to a particularly preferred embodiment of the present invention, the serum or plasma (lyophilized, liquid or frozen) is stored under vacuum or an inert atmosphere (preferably a noble gas, such as helium or argon, more preferably a higher molecular weight noble gas, and most preferably argon) prior to irradiation. According to an alternative preferred embodiment of the present invention, the serum or plasma is held under low pressure, to decrease the amount of gas, particularly oxygen and nitrogen, dissolved in the liquid, prior to irradiation, either with or without a prior step of solvent reduction, such as lyophilization. Such degassing may be performed using any of the methods known to one skilled in the art. For example, the serum or plasma may be treated prior to irradiation with at least one cycle, and preferably three cycles, of being subjected to a vacuum and then being placed under an atmosphere comprising at least one noble gas, such as argon, or nitrogen.

In another preferred embodiment, where the serum or plasma contain oxygen or other gases dissolved within the serum or plasma or within their container or associated with them, the amount of these gases within or associated with the preparation of serum or plasma may be reduced by any of the methods and techniques known and available to those skilled in the art, such as the controlled reduction of pressure within a container (rigid or flexible) holding the preparation of serum or plasma to be treated or by placing the preparation of serum or plasma in a container of approximately equal volume.

It will be appreciated that the combination of one or more of the features described herein may be employed to further minimize undesirable effects upon the serum or plasma caused by irradiation, while maintaining adequate effectiveness of the irradiation process on the biological contaminant(s) or pathogen(s). For example, in addition to the use of a stabilizer, the serum or plasma may also be lyophilized, held at a reduced temperature and/or kept under vacuum prior to irradiation to further minimize undesirable effects.

The sensitivity of a particular biological contaminant or pathogen to radiation is commonly calculated by determining the dose necessary to inactivate or kill all but 37% of the agent in a sample, which is known as the D₃₇ value. The desirable components of a serum or plasma may also be considered to have a D₃₇ value equal to the dose of radiation required to eliminate all but 37% of their desirable biological and physiological characteristics.

In accordance with certain preferred methods of the present invention, the sterilization of serum or plasma is conducted under conditions that result in a decrease in the D₃₇ value of the biological contaminant or pathogen without a concomitant decrease in the D₃₇ value of the serum or plasma. In accordance with other preferred methods of the present invention, the sterilization of serum or plasma is conducted under conditions that result in an increase in the D₃₇ value of the serum or plasma material. In accordance with the most preferred methods of the present invention, the sterilization of serum or plasma is conducted under conditions that result in a decrease in the D₃₇ value of the biological contaminant or pathogen and a concomitant increase in the D₃₇ value of the serum or plasma.

In accordance with certain preferred methods of the present invention, the sterilization of serum or plasma is conducted under conditions that reduce the possibility of the production of neo-antigens. In accordance with other preferred embodiments of the present invention, the sterilization of serum or plasma is conducted under conditions that result in the production of substantially no neo-antigens. The present invention also includes serum or plasmas sterilized according to such methods.

In accordance with certain preferred methods of the present invention, the sterilization of serum or plasma is conducted under conditions that reduce the total antigenicity of the serum or plasma(s). In accordance with other preferred embodiments of the present invention the sterilization of serum or plasma is conducted under conditions that reduce the number of reactive allo-antigens and/or xeno-antigens in the serum or plasma(s). The present invention also includes serum or plasmas sterilized according to such methods.

According to certain preferred embodiments of the present invention, serum or plasma sterilized according to the methods described herein may be introduced into a mammal in need thereof for prophylaxis or treatment of a condition or disease. Methods of introducing serum or plasma into a mammal are known to those skilled in the art.

According to certain preferred embodiments of the present invention, the turbidity of the serum or plasma is not increased following irradiation compared to a pre-irradiation value and is preferably decreased. Turbidity may be determined by one having ordinary skill in the art using methods and techniques known in the art. For instance, turbidity of the serum or plasma can be measured using a DRT-100B Turbidometer from HF Scientific, Inc., according to the manufacturers instructions, to give turbidity in NTU's (nominal turbidity units). In certain preferred embodiments of the present invention, the turbidity of the serum or plasma is less than 30 NTU following irradiation, mote preferably less than 25 NTU following irradiation, still more preferably less than 20 NTU following irradiation, even more preferably less than 15 NTU following irradiation, still even more preferably less than 10 NTU following irradiation, yet still more preferably less than 5 NTU following irradiation and most preferably less than 2.5 NTU following irradiation.

According to the embodiments of the present invention, the serum or plasma exhibits thermal stability about equal to the thermal stability of the serum or plasma prior to irradiation. Preferably, the serum or plasma has thermal stability following irradiation of up to 60° C., more preferably up to 55° C., still more preferably up to 56° C. and most preferably up to 60° C. or even higher.

According to certain preferred embodiments of the present invention, the serum or plasma is subjected to complement deactivation either before or after sterilization, preferably after sterilization. Complement activity can be reduced by methods and techniques known in the art such as by heating the serum or plasma to a temperature effective to reduce complement activity. According to certain preferred embodiments of the present invention, the serum or plasma is heated to not less than 50° C., more preferably not less than 55° C., even more preferably not less than 56° C., still more preferably not less than 57° C. and most preferably not less than 60° C. Suitable temperature may be determined empirically by one having ordinary skill in the art using methods and techniques known in the art. According to other embodiments compliment activity may be reduced by irradiation alone or a combination of irradiation and heating.

According to certain preferred embodiments of the present invention, the functional activity of the serum or plasma following sterilization is about 100% of the pre-irradiation value or even higher. In other preferred embodiments of the present invention, the functional activity of the serum or plasma following sterilization is at least 95% of the pre-irradiation value, at least 90% of the pre-irradiation value, at least 85% of the pre-irradiation value, at least 80% of the pre-irradiation value, at least 70% of the pre-irradiation value, at least 60% of the pre-irradiation value or at least 50% of the pre-irradiation value. According to the methods of the present invention, functional activity by methods and techniques known in the art, for instance, by a Rubella Ag-Reactive IgG assay, determining Factor VIII recovery, determining hemoglobin recovery of the serum or plasma, turbidity, supporting cell growth and protein integrity as shown for example by gel electrophoresis (e.g., absence of high molecular weight bands, absence of degradation products, etc.).

Other preferred embodiments of the present invention are directed to compositions containing serum or plasma sterilized according to the methods disclosed herein. Such compositions include, but are not limited to, serum or plasma treated with a single or plurality of stabilizing processes. Additionally, such compositions may be further treated or processed and may contain additional additives, supplements and the like.

According to certain preferred embodiments of the present invention, the irradiation of the biological material is performed under conditions whereby the temperature of said biological material increases during the irradiation from an initial temperature (Tᵢ) to a final temperature (T_{f}). Preferably, the increase in the temperature of the biological material (ΔT) is about equal to the total dose of radiation (D) divided by the specific heat capacity (c) of the biological material. Specific heat capacities of particular biological materials are known, or may be determined empirically by one skilled in the art using known methods and techniques.

Preferably, the final temperature (T_{f}) is at or below a level effective to protect the biological material from the radiation. According to such embodiments, the maximum acceptable temperature (Tₘₐₓ) for a particular biological material is preferably determined empirically by one skilled in the art employing the particular irradiation conditions desired. According to such embodiments of the present invention, the initial temperature (Tᵢ) of the biological material is then preferably set at a level at or below Tₘₐₓ-ΔT prior to irradiation.

According to other embodiments of the present invention, the increase in the temperature of the biological material is less than the total dose of radiation (D) divided by the specific heat capacity (c) of the biological material. Such variation may be due to the particular biological material being irradiated, the size of the sample being irradiated, the packaging in which the sample is contained, the particular method(s) of cooling, as well as the environment in which the package is held during irradiation. According to such embodiments, the increase in the temperature of the biological material (ΔT) is preferably determined empirically by one skilled in the art using known methods and techniques. The initial temperature (Tᵢ) of the biological material is then preferably set at a level at or below Tₘₐₓ-ΔT prior to irradiation.

### EXAMPLES

Having now fully described this invention, it will be understood to those of ordinary skill in the art that the methods of the present invention can be carried out with a wide and equivalent range of conditions, formulations and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

### Example 1

Purpose: To examine the effects of various stabilizers on Porcine Plasma FVIII Irradiated to about 50 kGy.

Materials:

1. Porcine Plasma FVIII Working Standard: Porcine Plasma, pig plasma with sodium citrate thawed and aliquot on Feb. 27, 2002 (Pel-Freeze Biological, code 39540-1, lot 31223, refer to exp#112601.RRC.086)

2. Porcine FVIII Standard (Hyate: C, Porcine Antihemophilic Factor, FVIII, from Ipsen, 585 U/ml, lot#608, exp. October 2000)

3. Pooled Fresh porcine plasma from USDA, pig#1-4

4. Histidine, Sigma H-8125, lot#91K0893

5. Sodium ascorbate, Spectrum S1349, lot#QS1127

6. Reagent Grade Water (NERL, lot#0305151, exp 03/2002)

7. 2 ml Glass Vials, Wheaton Cat#223683, lot#1199824-01

8. Stopper, Stelmi C1503, 6720GC 6 DT

9. Bovine Serum Albumin, Sigma, A-2153, lot#40K0896

10. Immunochrom FVIII: C Reagent Kit (Hylaland Immuno Diagnostic, lot#ON11000, exp. Dec. 31, 2002 and lot#ON12000, exp. Jun. 30, 2003)

11. Microwell Plate 96F Without Lid (Nunc, cat#269620)

12. Acetate Plate Sealers (DYNEX Technologies, Inc. cat#3501)

13. Solution Basins, 55 ml (Labcor Cat#730-001, vWR cat#21007-970)

14. Molecular Device Spectra MAX 190, Microplate Reader

15. Acetic Acid, GR Glacial, FW 60.05, EM Science AX0073-14, lot#128330700 043

16. Multichannel pipettors from Fisher

γ-Irradiation Condition:

Temperature: −72° C. (dry ice)

Procedure:

1. Preparation of Reagents:

0.5M Histidine: Sigma H-8125, Lot#91K0893 FW 209.6 Measured 6.29 g histidine and adjusted pH to 6.77, Q.S. with H₂O to 60 ml

2M Na-Asc: Spectrum S1394, Lot#QS1127, FW198.11 Measured 5.94 g sodium ascorbate and Q.S. with H₂O to 15 ml. (pH=7.79)

2. Setup y-Irradiation Samples With Fresh Porcine Plasma (Filtered and Unfiltered PRR and PPP)

After preparation of porcine plasma (PRP and PPP), took 25 ml of each filtered and unfiltered PRP and PPP setup for irradiation (take 140 ml of filtered PPP)

| | | | | | | Final | | | Time |
|---|---|---|---|---|---|---|---|---|---|
| | | | | Final | 2M | Conc. | | Dilution | Went |
| | | ml | 0.5M | Conc. | Na- | of | pH | Factor | in −80 C. |
| Sample | | of | His. | of | Asc | Na- | Before | for | Before |
| Description | Sample Code | Plasma | (ml) | His. | (ml) | Asc. | Forzen | Plasma | Irradiation |
| 21 ml Fresh | P/u/r/0a-d, | 11 | — | — | — | — | 7.54 | 1 | 4:20 pm |
| Unfiltered | 50a-d | | | | | | | | |
| Platelet | P/u/r/(H + A) | 10 | 2.66 | 100 | 0.67 | 100 | 6.61 | 1.333 | |
| Rich | 100/0a-d, 50a-d | | | | | | | | |
| Plasma | | | | | | | | | |
| (uPRP) | | | | | | | | | |
| 21 ml Fresh | P/u/p/0a-d, | 11 | — | — | — | — | 7.48 | 1 | 4:35 pm |
| Unfiltered | 50a-d | | | | | | | | |
| Platelet | P/u/p/(H + A) | 10 | 2.66 | 100 | 0.67 | 100 | 6.72 | 1.333 | |
| Poor | 100/0a-d, | | | | | | | | |
| Plasma | 50a-d | | | | | | | | |
| (uPPP) | | | | | | | | | |
| 21 ml Fresh | P/f/r/0a-d, | 11 | — | — | | | 7.55 | 1 | 4:50 pm |
| Filtered | 50a-d | | | | | | | | |
| Platelet | P/f/r/(H + A) | 10 | 2.66 | 100 | 0.67 | 100 | 6.60 | 1.333 | |
| Rich | 100/0a-d, 50a-d | | | | | | | | |
| Plasma | | | | | | | | | |
| (fPRP) | | | | | | | | | |
| 121 ml | P/f/p/0a-d, | 15 | — | — | — | — | 7.45 | 1 | 6:10 pm |
| Fresh | 50a-d | | | | | | | | |
| Filtered | P/f/p/H50/0a-d, | 10 | 1.11 | 50 | — | — | 6.73 | 1.11 | |
| Platelet | 50a-d | | | | | | | | |
| Poor | P/f/p/H100/ | 15 | 3.62 | 100 | — | — | 6.64 | 1.25 | |
| Plasma | 0a-d, 50a-d | | | | | | | | |
| (fPPP) | P/f/p/H200/ | 10 | 6.66 | 200 | — | — | 6.38 | 1.66 | |
| | 0a-d, 50a-d | | | | | | | | |
| | P/f/p/A100/ | 14 | — | — | 0.74 | 100 | 7.44 | 1.053 | |
| | 0a-d | | | | | | | | |
| | P/f/p/A200/ | 10 | — | — | 1.11 | 200 | 7.41 | 1.11 | |
| | 0a-d | | | | | | | | |
| | P/f/p/H50 + A100/ | 10 | 1.18 | 50 | 0.59 | 100 | 6.75 | 1.177 | |
| | 0a-d, | | | | | | | | |
| | 50a-d | | | | | | | | |
| | P/f/p/H50 + A200/ | 10 | 1.25 | 50 | 1.25 | 200 | 6.73 | 1.25 | |
| | 0a-d, | | | | | | | | |
| | 50a-d | | | | | | | | |
| | P/f/p/(H + A) | 14 | 3.72 | 100 | 0.94 | 100 | 6.59 | 1.333 | |
| | 100/0a-d, 50a-d | | | | | | | | |
| | P/f/p/H100 + A200/ | 10 | 2.9 | 100 | 1.43 | 200 | 6.60 | 1.333 | |
| | 0a-d, | | | | | | | | |
| | 50a-d | | | | | | | | |
| | P/f/p/H200 + A100/ | 10 | 7.27 | 200 | 0.91 | 100 | 6.53 | 1.433 | |
| | 0a-d, | | | | | | | | |
| | 50a-d | | | | | | | | |
| | P/f/p/(H + A) | 10 | 8 | 200 | 2 | 200 | 6.55 | 2 | |
| | 200/0a-d, 50a-d | | | | | | | | |

Aliquoted 1.2 plasma +/−additives into 2 ml glass vials

Used remaining plasma samples from each set of the condition to measure pH after finishing setting up samples. Froze samples in −80C freezer before going out for γ-irradiation.

FVIII Chromogenic Assay:

Preparation of Reagents:

Reagent A and B: Reconstituted each of a vial of Reagent A and Reagent B with 2 ml of Reagent Grade Water.

Substrate: Reconstituted the substrate in 2 ml of Reagent Grade Water. Then 1:5 dilution by adding 2 ml of substrate to 8 ml of FVIII Reaction Buffer.

Dilution Buffer: Added 2 ml 20% BSA to 50 ml Dilution Buffer to make 1% BSA in Dilution Buffer.

Preparation of Standard and Samples:

Predilutions of Porcine FVIII Standard (P8-(EDP)-Std):

Thawed 2 vials of Porcine FVIII Standard at RT. Made duplicate predilution samples for standard

Thawed one vial of Porcine FVIII Standard at RT. Made one predilution sample for standard

Made 1:10 predilutions with FDP, then another 1:10 dilutions with Dilution Buffer.

Predilutions of Porcine Plsama FVIII Working Standard (Pp-WStd-Std):

Thawed 2 vials of Porcine Plasma FVIII Working Standard in 37° C. water bath. Made duplicate predilution for each vial

Made 1:10 predilutions with Dilution Buffer, then another 1:10 dilutions with Dilution Buffer.

+/−γ-Irradiated Frozen Porcine Plasma:

Predilution: Thawed plasma in 37° C. water bath. Made 1:10 dilutions with Dilution Buffer

Final Dilutions for All Samples:

Made 1:41 dilution with Dilution Buffet (25 μl prediluted FVIII to 1 ml Dilution Buffer) in an eppendorf tube. 1:82, 1:164, 1:328 and 1:656 dilutions were made on a microtiter plate with Dilution Buffer

Assay:

Pre-warmed Reagent A and B, and Substrate in a 37° C. incubator

Added 20 μl FVIII to each well except for Reagent Blank Wells

Added 20 μl Dilution Buffer to each Reagent Blank well

Added 20 μl Reagent A and 20 μl Reagent B to each well

Shook the plate for 5 sec. and incubated at 37° C. for 5 minutes.

Added 100 μl of Substrate to each well

Shook the plate for 5 sec. and incubated at 37° C. for 5-15 minutes

(Assay was done in the microplate reader with 37C incubation)

Added 40 μl of 20% acetic acid to stop the reaction and read again

Results:

1. For the filtered PPP y-irradiated samples, the addition of 10 mM histidine and 200 mM sodium ascorbate gave the best FVIII activity recovery. The samples with 50 mM histidine had slightly higher potency after irradiation than the samples without any additives. The samples with 100 mM sodium ascorbate had higher FVIII potency after irradiation than the samples without any additives.

2. The addition of combination of 100 mM histidine and 10 mM sodium ascorbate had protective effects on porcine plasma (+/−filtration and +/−platelet). The unfiltered PPP samples with 100 mM histidine and sodium ascorbate had the highest potency amount of the irradiated samples (3.54 U/ml). The filtered PPP lost about 20% activity as compared to filtered PRP and unfiltered PPP and PRP.

### Example 2

Purpose: To determine what the recovery of FVIII is when Porcine Plasma is irradiated in the presence of Histidine and Sodium Ascorbate and/or when degassed and also when Monarc-M is irradiated in the presence of Na-Ascorbate and/or when degassed.

Materials:

Universal Reagents FVIII Depleted Plasma, Prod# FD80, lot# 0730079, exp Jul. 19, 2003. Thawed, dispensed into aliquots and frozen Mar. 7, 2002.

Clearant, Inc. FVIII Working Standard, lot # 002: American Red Cross FVIII, Antihemophilic Factor (Human), Method M, Monoclonal Purified, Monarc-M, lot# 29359024AA, exp. Apr. 4, 2001. 1050 U/bottle. Two bottles reconstituted on Aug. 21, 2001 with 10 ml water each, pooled and dispensed into 100 μl aliquots (conc. 105 U/ml, according to label) in 2 ml glass vials. Stored in −80° C. freezer. Calibrated against Mega-1 National FVIII Standard and assigned a potency of 88.2 U/ml.

Pel-Freeze Porcine Plasma, code 39540-1, lot 31223, aliquoted Feb. 27, 2002

USDA Porcine Plasma, platelet-poor, unfiltered, received and processed Mar. 19, 2002

APTT: Organon Teknika Platelin L Reagent APTT, lot# 131991, exp November/2003

CaCl₂: Organon Teknika Platelin L CaCk2, lot# 132006, exp June/2004

BAT Buffer: 50 mM Imidazole, 0.01%(v/v) Tween-20, 66 mM NaCl, made Apr. 15, 2002 & Apr. 24, 2002

Bovine Serum Albumin: Sigma, cat# A-2153, lot# 40K0896

Imidazole: Sigma, cat# 1-0125, lot# 60K0078

NaCl: J. T. Baker, cat# 4058-05

Tween-20: Thomas Scientific, cat# 0564k22, lot# H273610

Histidine: Sigma, cat# H-8125, lot# 91K0893, FW 209.6

Sodium Ascorbate: Spectrum, cat# S1394, lot# QS1127, FW 198.11

Reagent Grade Water: Nerl cat# 9800-5, lot# 0305151, exp March/2002

In-house filtered water (Millipore): for making BAT buffer

USP Sterile Water (for reconstituting FVIII): Baxter, cat# 3476, lot# 114415, February/03

Microcentrifuge tubes: Costar, cat# 3620, 1.7 ml (polypropylene) VWR, cat# 20170-128, 2.0 ml (polypropylene)

Conical tubes: 15 ml & 50 ml (polypropylene)

Gilson Pipettman (P-20, P-100, P-200, P-1000)

MLA Electra 1400C

MLA materials: sample cups, reagent cups, cuvettes, etc

2 ml glass vials: Wheaton, cat# 223683, lot# 1206540-01

Rubber stoppers: Stelmi, cat# C1503, lot# G006/5511

Set-Up:

1) Stock reagents:

2M Sodium Ascorbate:

1.9811 gNa-Asc dissolved in water to 5 ml. (pH=7.53)

2.5M Sodium Ascorbate:

2.476 g Na-Asc dissolved in water to 5 ml. (pH=7.67)

0.5M Histidine:

1.048 g Histidine dissolved in water to 10 ml, applying heat. (pH=6.95)

2) Prepared the following solutions:

| | | | 2.5M | | | |
|---|---|---|---|---|---|---|
| | | | Na- | 0.5M | pH | Plasma |
| Sample | Sample | ml of | Asc | His | before | dilution |
| Description | Code | Plasma | (ml) | (ml) | freezing | factor |
| Unfiltered | P/u/p/0a-f, | 14.4 | — | — | 7.93 | 1 |
| PPP | 50a-f | | | | | |
| Unfiltered | P/u/p/(H + A) | 12 | 0.61 | 3.19 | 7.26 | 1.317 |
| PPP + 100 mM | 100/ | | | | | |
| both | 0a-f, 50a-f | | | | | |
| His & Na- | | | | | | |
| Asc | | | | | | |
| Unfiltered | d/P/u/p/0a-f, | 14.4 | — | — | 7.93 | 1 |
| PPP | 50a-f | | | | | |
| (samples to | | | | | | |
| be | | | | | | |
| degassed) | | | | | | |
| Unfiltered | d/P/u/p/(H + A) | 12 | 0.61 | 3.19 | 7.26 | 1.317 |
| PPP + 100 mM | 100/0a-f, | | | | | |
| both | 50a-f | | | | | |
| His & Na-Asc | | | | | | |
| (samples to | | | | | | |
| be | | | | | | |
| degassed) | | | | | | |
| | | | 2M | | | |
| | | | Na- | | pH | Plasma |
| Sample | Sample | ml of | Asc | Water | before | Dilution |
| Description | Code | Conc. | (ml) | (ml) | freezing | factor |
| Monarc-M | M/0a-f, 50a-f | 2.5 | — | 0.28 | 7.24 | 1.11 |
| Monarc-M + 200 mM | M/A200/0a-f, | 2.5 | 0.28 | — | 7.42 | 1.11 |
| Na- | 50a-f | | | | | |
| Asc | | | | | | |
| Monarc-M | d/M/0a-f, | 2.5 | — | 0.28 | 7.24 | 1.11 |
| (samples to | 50a-f | | | | | |
| be degassed) | (samples to | | | | | |
| | be degassed) | | | | | |
| Monarc-M + 200 mM | d/M/A200/0a-f, | 2.5 | 0.28 | — | 7.42 | 1.11 |
| Na- | 50a-f | | | | | |
| Asc | (samples to | | | | | |
| (samples to | be degassed) | | | | | |
| be | | | | | | |
| degassed) | | | | | | |

3) For the Porcine plasma, aliquoted 1.2 ml from each solution into 2 ml glass vials and covered with a rubber stopper. For the Monarc-M concentrate, aliquoted 0.2 ml. Samples that were not be degassed were crimped and kept at the same temperature as the samples to be degassed (room temperature).

Degassing:

Samples to be degassed were placed into the freeze dryer and degassed in the following manner:

1) The condenser was cooled to −80° C. and the vials were placed inside.

2) The vacuum switch was turned on and vacuum applied to the chamber for 90 seconds, the pressure (rough vacuum) inside the chamber was between about 25 mb and about 40 mb.

3) After 90 seconds, the vacuum was turned off, the chamber vent tube was connected with Argon gas at 2 psi and the vacuum released for 5 minutes to allow the chamber to fill with Argon.

4) This process was repeated 3 times to ensure that all of the air has been removed form the chamber.

5) At the end of the third degassing step, vacuum was applied for 90 seconds at the end of which the vacuum was turned off and the vials sealed.

6) The vials were removed and crimped closed.

7) Froze all vials, including those not degassed, at −80° C.

Irradiation:

Samples were irradiated to about 50 kGy with gamma radiation.

Method:

1) Added 1% BSA to BAT buffer.

2) Thawed FVIII deficient plasma in a warm water bath.

3) Prepared the MLA (lamp calibration, confidence test, add CaCl₂ and APTT, etc) for assay.

4) Thawed an aliquot of the working std at RT.

5) Prediluted in duplicate 1:88.2 by first diluting 1:8.82 (23 μl FVIII+180 μl FDP) and again 1:10 (80 μl first dilution+720 ml FDP) and assayed (7 dilution points).

6) Thawed two aliquots of the Pel-Freeze Porcine plasma and assayed.

7) Assayed the samples four at a time by first removing four vials form −80° C., thawing in a 37° C. water bath for 2 min., transferring plasma to a sample cup and then placing in the MLA.

8) Measured the pH of each plasma sample after assayed by MLA.

Results/Conclusions:

Degassed porcine plasma showed a recovery of 77% compared to 74% for samples not degassed.

Degassed porcine plasma plus histidine & ascorbate showed a recovery of 81% compared to 82% samples not degassed.

Degassed Monarc-M showed a recovery of 63% compared to 59% for samples not degassed.

Degassed Monarc-M plus histidine & ascorbate showed a recovery of 59% compared to 58% for samples not degassed.

### Example 3

Purpose: To examine the effects of gamma irradiation of porcine plasma FVIII at various temperature with or with the presence of various stabilizers.

Materials:

17. MLA Electra 1400C Automatic Coagulation Analyzer (Hemoliance, serial #1395CE, system ID#411503)

18. APTT-Platelin L from Organon Teknika (lot#'s131973 and 131991)

19. Platelin L CaCl₂ (CaCl₂-L), Organon Teknika (lot#131974 and lot#132006)

20. FVIII Artificially Depleted Human Source Plasma from Universal Reagents (lot#0730079)

21. Reagent Grade Water (NERL, lot#0305151)

22. BAT Buffer: 50 nM Imidazole, 66 mM NaCl, 0.01% (v/v) Tween-20, 1% BSA (added upon use), pH 7.4.

23. Imidazole (Sigma, 1-0125, lot#60K0078)

24. Sodium Chloride (vWR, #vW6430-7)

25. Tween 20 (EM science OmniPur Tween 20 (100%, MW 1163.92, lot#3630B98)

26. Bovine Serum Albumin (Signa, A-2153, lot#401K0896)

27. Sodium Ascorbate (Aldrich Chemicals, lot#10801HU, 26,855-0 [134-03-2])

28. FVIII Working Standard: 92.4 U/ml.

29. Porcine Plasma, pig plasma with sodium citrate (Pel-Freeze Biological, code 39540-1, lot 31223)

30. Polypropylene Tubes, 1.5 ml disposable screw cap tube with skirt and cap, sterile lot#6029, vWR cat#20170-235

31. 2 ml Glass Vials, Wheaton Cat#223683, lot#1178013-01

32. Stopper, Stelmi C1503, 6720GC 6 DT, lot# N:G006/5511

33. Corning 500 ml Filter System (Corning cat#431097, 0.22 μm polyethersulfone, sterilizing, low protein membrane, ploystyrene)

Irradiation Conditiona:

| Dose Rate: | High | Medium | Low |
|---|---|---|---|
| Dose Rate: | 4.982 kGy/hr | 1.505 kGy/hr | ˜0.2 kGy/hr |
| Total Dose: | 50 kGy | 50 kGy | 50 kGy |
| Temperature: | dry ice | dry ice | dry ice |

Procedure:

Setup Porcine Plasma Samples for γ-Irradiation

1. Thawed Plasma

Took Porcine Plasma out from the −80° C. freezer and left in refrigerator overnight Next morning, took plasma container out and put in a 37° C. water bath. Let the plasma thaw completely and allowed the temperature of plasma to reach about 20.5° C.

2. Setup Plasma Samples +/−Asc

1) Plasma Setup in Glass Vials:

a) Frozen at −80° C. before being sent out for gamma irradiation

−Ascorbate: P/H/0a-c, P/M/0a-c, P/L/0a-c, P/H/50a-c, P/M/50a-c, P/L/50a-c

+10 mM Ascorbate: P/H/10A/0a-c, P/M/10A/0a-c, P/L/10A/0a-c, P/H/10A/50a-c, P/M/10A/50a-c, P/L/10A/45a-c

+50 mM Ascorbate: P/H/50A/0a-c, P/M/50A/0a-c, P/L/50A/0a-c, P/H/50A/45a-c, P/M/50A/50a-c, P/L/50A/50a-c

+100 mM Ascorbate: P/H/100A/0a-c, P/M/100A/0a-c, P/L/100A/0a-c, P/H/100A/50a-c, P/M/100A/50a-c, P/L/100A/50a-c

+200 mM Ascorbate: P/H/200A/0a-c, P/M/200A/0a-c, P/L/200A/0a-c, P/H/200A/50a-c, P/M/200A/50a-c, P/L/200A/50a-c

Control: P/Ctrl/a-c (−Asc), P/ctrl/10A/a-c, P/ctrl/50A/a-c, P/ctrl/100A/ac, and P/ctrl/200A/a-c (+Asc) (Control stored at −80° C.)

b) Frozen in Liquid Nitrogen before sent out for gamma irradiation

−Ascorbate: P/M/LN/0a-c, P/M/LN/50a-c

+100 mM Ascorbate: P/M/LN/100A/0a-c, P/M/LN/100A/50a-c

2) Plasma Setup in Polypropylene Vials:

Ascorbate: P/M/P/0a-c, P/M/P/50a-c

+100 mM Ascorbate: P/M/P/100A/0a-c, P/M/P/100A/50a-c

Note:

1. Porcine plasma pH=7.5

2. 2M Na-Asc pH=7.7

| | | | | Final | | | | |
|---|---|---|---|---|---|---|---|---|
| | | | μl | Conc | | γ- | | |
| | | μl of | 2M | Na- | Final | Cell | Dose | Total |
| | Sample | Plasma/ | Na | Asc | Vol. | Temp. | Rate | Dose |
| Sample Code | Description | Vial | Asc | (mM) | (μl) | (° C.) | kGy/hr | (kGy) |
| P/Ctrl/a-c | Porcine Plasma | 1000 | | 0 | 1000 | −80° C. | 0 | — |
| P/H/(+/−)50 kGy a-c | Samples w/o | 1000 | | 0 | 1000 | −80° C. | ˜5 | +/−50 |
| P/M/(+/−)50 kGy a-c | Na-Asc Frozen | 1000 | | 0 | 1000 | −80° C. | ˜1.5 | +/−50 |
| P/L/(+/−)50 kGy a-c | in Glass Vials | 1000 | | 0 | 1000 | −80° C. | ˜0.2 | +/−50 |
| P/ctrl/10A/a-c | Porcine Plasma | 1000 | 5 | 10 | 1005 | −80° C. | 0 | — |
| P/H/10A/(+/−)50 kGy | Samples w/ | 1000 | 5 | 10 | 1005 | −80° C. | ˜5 | +/−50 |
| a-c | 10 mM Na-Asc | | | | | | | |
| P/M/10A/(+/−)50 kGy | Frozen in Glass | 1000 | 5 | 10 | 1005 | −80° C. | ˜1.5 | +/−50 |
| a-c | Vials | | | | | | | |
| P/L/10A/(+/−)50 kGy | | 1000 | 5 | 10 | 1005 | −80° C. | ˜0.2 | +/−50 |
| a-c | | | | | | | | |
| P/ctrl/50A/a-c | Porcine Plasma | 1000 | 25.6 | 50 | 1025.6 | −80° C. | 0 | — |
| P/H/50A/(+/−)50 kGy | Samples w/ | 1000 | 25.6 | 50 | 1025.6 | −80° C. | ˜5 | +/−50 |
| a-c | 50 mM Na-Asc | | | | | | | |
| P/M/50A/(+/−)50 kGy | Frozen in Glass | 1000 | 25.6 | 50 | 1025.6 | −80° C. | ˜1.5 | +/−50 |
| a-c | Vials | | | | | | | |
| P/L/50A/(+/−)50 kGy | | 1000 | 25.6 | 50 | 1025.6 | −80° C. | ˜0.2 | +/−50 |
| a-c | | | | | | | | |
| P/ctrl/100A/a-c | Porcine Plasma | 1000 | 52.6 | 100 | 1052.6 | −80° C. | 0 | — |
| P/H/100A/(+/−) | Samples w/ | 1000 | 52.6 | 100 | 1052.6 | −80° C. | ˜5 | +/−50 |
| 50 kGy a-c | 100 mM Na-Asc | | | | | | | |
| P/M/100A/(+/−) | Frozen in Glass | 1000 | 52.6 | 100 | 1052.6 | −80° C. | ˜1.5 | +/−50 |
| 50 kGy a-c | Vials | | | | | | | |
| P/L/100A/(+/−)50 kGy | | 1000 | 52.6 | 100 | 1052.6 | −80° C. | ˜0.2 | +/−50 |
| a-c | | | | | | | | |
| P/ctrl/200A/a-c | Porcine Plasma | 1000 | 111 | 200 | 1111 | −80° C. | 0 | — |
| P/H/200A/(+/−) | Samples w/ | 1000 | 111 | 200 | 1111 | −80° C. | ˜5 | +/−50 |
| 50 kGy a-c | 200 mM Na-Asc | | | | | | | |
| P/M/200A/(+/−) | Frozen in Glass | 1000 | 111 | 200 | 1111 | −80° C. | ˜1.5 | +/−50 |
| 50 kGy a-c | Vials | | | | | | | |
| P/L/200A/(+/−)50 kGy | | 1000 | 111 | 200 | 1111 | −80° C. | ˜0.2 | +/−50 |
| a-c | | | | | | | | |
| P/M/LN/(+/−)50 kGy | Porcine Plasma | 1000 | 52.6 | 100 | 1052.6 | −80° C. | ˜1.5 | +/−50 |
| a-c | Samples (+/−) | | | | | | | |
| P/M/LN/100A/(+/−) | Na-Asc Frozen | 1000 | 52.6 | 100 | 1052.6 | −80° C. | ˜1.5 | +/−50 |
| 50 kGy a-c | in Liquid | | | | | | | |
| | Nitrogen | | | | | | | |
| P/P/ctrl/100A/a-c | Porcine Plasma | 1000 | 52.6 | 100 | 1052.6 | −80° C. | 0 | — |
| P/P/M/(+/−)50 kGy a-c | Samples (+/−) | 1000 | 52.6 | 100 | 1052.6 | −80° C. | ˜1.5 | +/−50 |
| P/P/M/100A/(+/−) | Na-Asc Frozen | 1000 | 52.6 | 100 | 1052.6 | −80° C. | ˜1.5 | +/−50 |
| 50 kGy a-c | in | | | | | | | |
| | Polypropylene | | | | | | | |
| | Vials | | | | | | | |

FVIII One-Stage Clotting Assay:

Preparation of Standard and Samples:

1. Predilution of FVIII Working Standard: Made 1:92.4 predilution of the FVIII with FDP.

20 μl of FVIII and 164.8 μl FDP

80 μl 1:9.24dil FVIII+720 μl FDP

Made duplicate predilutions: Std-1 and Std-2

2. Porcine Plasma Samples: Thawed plasma at 37° C. in a water bath for 2-3 min and performed clotting assays

Results:

The comparison of FVIII activity recoveries of porcine plasma samples with ascorbate concentration from 0-200 mM after irradiation on dry ice at different dose rates are listed in Table 1.

TABLE 1

| | | | Dose Rate | | Dose Rate | |
|---|---|---|---|---|---|---|
| | | | 4.082 kGy/hr | | 1.505 kGy/hr | |
| | Sodium | Irradiation | FVIII | FVIII | FVIII | FVIII |
| | Ascorbate | Received | Potency | Recovery | Potency | Recovery |
| Sample ID | Conc. (mM) | (kGy) | (U/ml) | (45/0 kGy) | (U/ml) | (45/0 kGy) |
| P/0 | 0 | 0 | 7.46 | | 7.13 | |
| P/50 | | 50 | 5.39 | 72.3% | 4.98 | 69.9% |
| P/10A/0 | 10 | 0 | 7.42 | | 7.11 | |
| P/10A/50 | | 50 | 5.37 | 72.5% | 5.36 | 75.3% |
| P/50A/0 | 50 | 0 | 7.43 | | 8.60 | |
| P/50A/50 | | 50 | 5.45 | 73.4% | 5.76 | 67.0% |
| P/100A/0 | 100 | 0 | 7.64 | | 7.82 | |
| P/100A/50 | | 50 | 5.76 | 75.4% | 5.87 | 75.0% |
| P/200A/0 | 200 | 0 | 7.77 | | 7.62 | |
| P/200A/50 | | 50 | 5.89 | 75.7% | 5.45 | 71.6% |

3. Table 1 shows that at medium dose rate higher Na-Asc concentrations (100-200 mM) give slightly better recovery.

4. The comparison of FVIII activity recoveries of porcine plasma samples frozen in liquid nitrogen and −80° C. freezer, the samples irradiated in glass vials and plastic vials are listed in Table 2.

TABLE 2

| | | | Freezing Method/Tube Type | | | | | |
|---|---|---|---|---|---|---|---|---|
| | | | −80° C. | | Liquid | | −80° C. | |
| | | | Freezer/Glass | | Nitrogen/Glass | | Freezer/Plastic | |
| | Na- | Irradiation | FVIII | FVIII | FVIII | FVIII | FVIII | FVIII |
| | Asc | Received | Potency | Recovery | Potency | Recovery | Potency | Recovery |
| SampleID | Conc | (kGy) | (U/ml) | (50/0 kGy) | (U/ml) | (50/0 kGy) | (U/ml) | (50/0 kGy) |
| P/(P)/(LN)/M | 0 | 0 | 7.13 | | 7.38 | | 7.51 | |
| | | 50 | 4.98 | 69.9% | 5.32 | 72.1% | 5.22 | 69.5% |
| P/(P)/(LN)/M/ | 100 | 0 | 7.82 | | 7.65 | | 7.53 | |
| 100A | | 50 | 5.87 | 75.0% | 5.6 | 73.1% | 5.55 | 73.3% |

Table 2 shows that at medium dose rate the plasma samples without Na-Asc had slightly better recovery when frozen in liquid nitrogen.
When 100 mM Na-Asc is added the recovery of the samples frozen in −80° C. freezer has slightly higher recovery.

### Example 4

Purpose: To examiner the effect of gamma irradiation on FVIII at varying pH values.

Materials:

Universal Reagents FVIII Depleted Plasma, Prod# FD80, lot# 0730079.

Clearant, Inc. FVIII Working Standard, lot # 003: American Red Cross FVIII: Antihemophilic Factor (Human), Method M, Monoclonal Purified, Monarc-M, lot# 29359024AA (4050 U/bottle). Two bottles reconstituted with 10 ml water each, pooled and dispensed into 100 μl aliquots (conc. 105 U/ml, according to label) in polypropylene tubes. Stored in −80° C. freezer. Calibrated against Mega-1 National FVIII Standard and assigned a potency of 102 U/ml.

Pel-Freeze Porcine Plasma, code 39540-1, lot 31223

USDA Unfiltered, Platelet Poor Porcine Plasma

11.6 M HCl

APTT: Organon Teknika Platelin L Reagent APTT, lot# 131991

CaCl₂: Organon Teknika Platelin L CaCl₂, lot# 132006

BAT Buffer: 50 mM Imidazole, 0.01%(v/v) Tween-20, 66 mM NaCl

Bovine Serum Albumin: Sigma, cat# A-2153, lot# 111K1654.

Imidazole: Sigma, cat# I-0125, lot# 60K0078

NaCl: J. T. Baker, cat# 4058-05

Tween-20: Thomas Scientific, cat# 0564k22, lot# H273610

MilliQ In-house filtered water (Millipore)

Sodium Citrate: Sigma, cat# S4641, lot# 61K0109

Microcentrifuge tubes: Costar, cat# 3620, 1.7 ml (polypropylene)

Conical tubes: 15 ml & 50 ml (polypropylene)

Gilson Pipettman (P-20, P-100, P-200, P-1000)

MLA Electra 1400C

MLA materials: sample cups, reagent cups, cuvettes, etc

2 ml glass vials: Wheaton, cat# 223683, lot# 1214767-01

Rubber stoppers: Stelmni, cat# 6720GC, lot# G009/7125

Set-Up:

1. Did a titration curve to determine the amounts of acid needed to obtain the desired pH level.

2. Made stock solutions

3. Thawed USDA unfiltered, plt-poor plasma and made 12 ml aliquots

4. Adjusted the pH while stirring, one aliquot at a time then calculated the amount of saline needed to bring all aliquots to the same volume.

5. 1 ml of plasma was placed in the appropriately labeled glass vial, covered with tubber stoppers, capped, crimped and placed in −80° C. freezer.

| | | Volume | Volume | | |
|---|---|---|---|---|---|
| | Volume | 0.2 M | 0.9 M | | Total |
| Target | Plasma | HCl | NaCl | Measured | Volume |
| pH | (ml) | (ml) | (ml) | pH | (ml) |
| 7.3 | 12 | 0.6 | 1.95 | 7.32 | 14.55 |
| 6.9 | 12 | 1.23 | 1.32 | 6.8 | 14.55 |
| 6.5 | 12 | 1.77 | 0.78 | 6.4 | 14.55 |
| 6.1 | 12 | 2.19 | 0.36 | 6.02 | 14.55 |
| 5.7 | 12 | 2.55 | — | 5.8 | 14.55 |
| Normal | 12 | — | 2.55 | 8.02 | 14.55 |
| Normal | 12 | — | | 8.03 | 12 |

Irradiation:

| | | Dose | |
|---|---|---|---|
| | | Specified | |
| Experiment ID | Sample Description | (kGy) | Temperature |
| 082002rwa.131.004 | Bovine Thrombin, +/− Asc, | 50 | dry ice |
| | +/− vaccinia, +/− PPV | | |
| 082702rwa.131.005 | Sucrose + Asc, + Gly-Gly | 50 | ambient |
| 082702.ccs.092.160 | PPF powder for various | 10, 30, 50 | dry ice |
| | doses | | |
| 082702.dla.35 | B19 clone in E. coli plasmid vector | 50 | dry ice |
| | in TE-Na Asc | | |
| | | 50 | dry ice |
| 082702.GPB.118.058 | Albumin +/− sucrose, +/− asc, | 50 | dry ice and |
| | +/− lactose | | ambient |
| 082302.IBV.102.103-087 | Porcine plasma | 50 | dry ice |
| CLS 4021, | PPV spiked human plasma, | 10, 30, 50 | dry ice |
| NB121 | +/− His, Asc | | |
| 081302.nbl.081 | Fraction V +/− surfactants | 50 | dry ice |
| 082702.slf.122.102 | Porcine ACL (untreated) | 50 | dry ice |
| 082702.xiy.135.001 | PPF +/− butanol (paste) | 50 | dry ice |

Irradiation Dosimetry Report

| | | Absorbed | | | | |
|---|---|---|---|---|---|---|
| | | Dose | | Dose | | |
| Irradiation | | Measured | | rate | | |
| Lot | | (kGy)* | | (kGy/hr) | | |
| Number | Canister ID | Min. | Max. | Min. | Max. | Temp. |
| 08210942 | 10A | 10.9 | 11 | 2.31 | 2.32 | dry ice |
| 08210943 | 30A | 30.4 | 31.9 | 2.12 | 2.22 | dry ice |
| 08210944 | 50A | 53.8 | 59.9 | 2.1 | 2.34 | dry ice |
| 08210945 | 50B | 51.5 | 54.1 | 2.04 | 2.14 | dry ice |
| 08210946 | 50C | 55.8 | 64.5 | 2.48 | 2.87 | ambient |

Method:

9) Added 1% BSA to BAT buffer.

10) Thawed FVIII deficient plasma in a 37° C. water bath.

11) Prepared the MLA (lamp calibration, confidence test, add CaCl₂ and APTT, etc) for assay.

12) Thawed an aliquot of FVIII working std lot# 003 at RT. Prediluted in duplicate 1:102 by first diluting 1:10.2 (20 μl FVIII+184 μl FDP) and again 1:10 (80 μl first dilution+720 ml FDP) and assayed (7 dilution points).

13) Thawed two aliquots of Pel-Freeze porcine plasma working std in a 37° C. water bath and assayed (7 dilution points)

14) Thawed the samples four at a time in a 37° C. water bath and assayed (4 dilution points).

15) After assay, measured the pH of the samples.

Results and Conclusions:

In this experiment, the best percent recovery, 75%, was seen in the undiluted plasma. The other percent recoveries ranged from 66%-71%.

### Example 5

Purpose: To examine the effects of gamma irradiation in FVIII irradiated to a total dose of 50 kGy at a dose rate of about 2 kGy/hr in the presence or absence of stablizers.

Materials:

34. Porcine Plasma, pig plasma with sodium citrate aliquot on Nov. 26, 2001 (Pel-Freeze Biological, code 39540-1, lot 31223)

35. Histidine, Sigma L-8125, lot#91K0893

36. Calcium Chloride, Sigma C-8106, lot#70K1852

37. MLA Electra 1400C Automatic Coagulation Analyzer (Hemoliance, serial #1395CE, system ID#411503)

38. APTT-Platelin L from Organon Teknika

39. Platelin L CaCl₂ (CaCl₂-L), Organon Teknika

40. FVIII Artificially Depleted Human Source Plasma from Universal Reagents (lot#0730079)

41. Reagent Grade Water NERL, lot#0305151)

42. BAT Buffer: 50 mM Imidazole, 66 mM NaCl, 0.01% (v/v) Tween-20, 1% BSA (added upon use), pH 7.4

43. FVIII Working Standard Lot#002: 88.2 U/ml, refer to exp#013102.RRC.094

44. 2 ml Glass Vials, Wheaton Cat#223683, lot#1178013-01

45. Stopper, Stelmi C1503, 6720GC 6 DT, lot# N:G006/5511

46. Centrifuge, Beckman GS-6R

Irradiation Conditions:

Dose Rate: 2.5-3.1 kGy/hr

Total Dose: 53.9-64.4 kGy

Temperature: −80° C. (dry ice)

Procedure:

3. Preparation of reagents:

a) 0.5M Histidine: 0.52 g histidine in H₂O to final volume=5 ml, pH=6.52

b) 1M Calcium Chloride: 3.55 g CaCl₂ in H₂O to final volume=24.15 ml 100 mM CaCl₂: Made 1:10 dilution of 1M CaCl₂

c) 0.5M Histidine+1 mM CaCl₂: 0.26 g histidine, 25 μl 100 mM CaCl₂ to final volume 2.5 ml, pH=6.53

d) 0.5M Histidine+10 mM CaCl₂: 0.26 g histidine, 25 μl 1M CaCl₂ to final volume 2.5 ml, pH=6.62

4. Thaw Frozen Porcine Plasma:

a) Cryo-poor plasma (supernatant of cryoptecipitation): Take 1 tube of 50 ml frozen porcine plasma out from the −80° C. freezer and thaw it on ice, centrifuge at 3° C., 3480 rpm for 5′, collect supernatant into 4×10 ml and 2×1.2 ml for controls

b) Cryo-rich plasma (without cryoprecipitation): Take 1 tube of 50 ml frozen porcine plasma out from the −80° C. freezer and thaw it in the 37° C. water bath. Let the plasma thaw completely and allow the temperature of plasma reach about 15° C. (temp=20.5° C.), aliquot plasma into 4×10 ml and 2×1.2 ml for controls

5. Setup Plasma Samples for Irradiation:

| | | | | | | | PH |
|---|---|---|---|---|---|---|---|
| | | Cryo- | | | Final conc. | | Before |
| Group | Samples ID | ppt | Plasma | Buffer | Histidine | CaCl₂ | Frozen |
| 1 | P/0a-d, P/50a-d | Cyo- | 12 ml | 1.33 ml H₂O | — | — | 7.64 |
| 2 | P/H/0a-d, | Rich | 12 ml | 1.33 ml 0.5M | 50 mM | — | 6.89 |
| | P/H/50a-d | | | Histidine | | | |
| 3 | P/H + C(0.1)/0a-d, | | 12 ml | 1.33 ml 0.5M | 50 mM | 0.1 mM | 6.90 |
| | P/H + C(0.1)/50a-d | | | Histidine + 1 mM | | | |
| | | | | CaCl₂ | | | |
| 4 | P/H + C(1)/0a-d, | | 12 ml | 1.33 ml 0.5M | 50 mM | 1 mM | 6.86 |
| | P/H + C(1)/50a-d | | | Histidine + 10 mM | | | |
| | | | | CaCl₂ | | | |
| 1 | P/Cyp/0a-d, | Cyo- | 10 ml | 1.1 ml H₂O | — | — | 7.52 |
| | P/Cyp/50a-d | Poor | | | | | |
| 2 | P/Cyp/H/0a-d, | | 10 ml | 1.1 ml 0.5M | 50 mM | — | 6.85 |
| | P/Cyp/H/50a-d | | | Histidine | | | |
| 3 | P/Cyp/H + C(0.1)/ | | 10 ml | 1.1 ml 0.5M | 50 mM | 0.1 mM | 6.90 |
| | 0a-d, | | | Histidine + 1 mM | | | |
| | P/Cyp/H + C(0.1)/ | | | CaCl₂ | | | |
| | 50a-d | | | | | | |
| 4 | P/Cyp/H + C(1)/ | | 10 ml | 1.1 ml 0.5M | 50 mM | 1 mM | 6.94 |
| | 0a-d, | | | Histidine + 10 mM | | | |
| | P/Cyp/H + C(1)/ | | | CaCl₂ | | | |
| | 50a-d | | | | | | |

Aliquot samples 1.2 ml/vial in 2 ml glass vials. Store samples in −80° C. freezer for irradiation.

Samples were analyzed by a One-Stage Clotting Assay, as discussed above.

Results:

5. The supernatant of cryo precipitation from frozen porcine plasma (cryo-poor) irradiated on dry ice showed slightly lower recovery than cryo-rich plasma when comparing the irradiated samples to the unirradiated samples for each condition (50/0 kGy).

6. The additions of histidine and histidine plus calcium chloride improved FVIII recovery after irradiation in cryo-rich plasma.

The cryo-rich samples with calcium chloride showed slightly improved recovery. Samples with 1 mM calcium chloride had slightly higher activity than samples with 0.1 mM calcium chloride.

### Example 6

Purpose:

To examiner the effects of gamma irradiation on porcine plasma FVIII irradiated to a total dose of about 10, about 30, or about 50 kGy.

Materials:

Universal Reagents FVIII Depleted Plasma, Prod# FD80, lot# 0730079

Hyate C Porcine FVIII Concentrate: Ipsen, lot# 608, 585 U/bottle. Reconstituted 2 bottles with 5.85 ml water each, pooled and aliquoted.

Pel-Freeze Porcine Plasma, code 39540-1, lot 31223

USDA Unfiltered, Platelet Poor Porcine Plasma

APTT: Organon Teknika Platelin L Reagent APTT

CaCl₂: Organon Teknika Platelin L CaCl₂

BAT Buffer: 50 mM Imidazole, 0.01%(v/v) Tween-20, 66 mM NaCl

Bovine Serum Albumin: Sigma, cat# A-2153, lot# 40K0896 or lot # 111 1654.

Imidazole: Sigma, cat# 1-0125, lot# 60K0078

NaCl: J. T. Baker, cat# 4058-05

Tween-20: Thomas Scientific, cat# 0564k22, lot# H273610

Reagent Grade Water: Nerl cat# 9800-5, lot# 0305151, exp March/2002

In-house filtered water (Millipore): for making BAT buffer

Microcentrifuge tubes: Costar, cat# 3620, 1.7 ml (polypropylene) and VWR, cat# 20170-128, 2.0 ml (polypropylene)

Conical tubes: 15 ml & 50 ml (polypropylene)

Gilson Pipettman (P-20, P-100, P-200, P-1000)

MLA Electra 1400C

MLA materials: sample cups, reagent cups, cuvettes, etc

Irradiation:

Samples were irradiated with gamma radiation to total doses of about 10, about 30 or about 50 kGy.

Method:

16) Added 1% BSA to BAT buffer.

17) Thawed FVIII deficient plasma in a warm water bath.

18) Prepared the MLA (lamp calibration, confidence test, add CaCl₂ and APTT, etc) for assay.

19) Thawed an aliquot of Hyate C at RT.

20)Prediluted in duplicate 1:100 by first diluting 1:10 (20 μl FVIII+180 μl FDP) and again 1:10 (801 first dilution+720 ml FDP) and assayed (7 dilution points).

21) Thawed 2 aliquots of Pel-Freeze Porcine Plasma in a 37° C. water bath and assayed.

22) Assayed the samples four at a time by first diluting 1001 of sample plasma in 500 wl FDP in a sample cup and then placing in the MLA.

Results:

The greatest recovery of FVIII following irradiation was seen in samples containing 100 mM His & Asc. The addition of 100 mM Asc alone also improved recovery. Percent recovery of mid-eighties was maintained both at 30 kGy and 50 kGy in the presence of both His & Asc. Recovery was in the upper sixties in samples containing no additives. In samples containing 100 mM ascorbate, recovery was in the mid-seventies.

### Example 7

Objectives: To examine the effects of gamma irradiation at various temperatures, including ambient temperature and below, on fetal bovine serum obtained from various commercial sources in the presence or absence of stabilizers as measured by various analytical methods, including turbidity, DSC, SDS-PAGE and HPLC. Exemplary stabilizer concentrations include: 50 mM pyruvate; and 50 mM L-histidine (pH 6.0) in combination with 10 mM sodium ascorbate. Additionally, to examine the effects of performing complement heat inactivation prior to and after irradiation as analyzed by analytical methods including turbidity, DSC profiles, and SDS-PAGE.

Examples were carried out within the scope of the following protocol:

### Materials

Chemicals

| Item | Manufacturer | Item Number |
|---|---|---|
| Water (Sterile) | Quality Biological. | Cat # 118-162-100, Lot # 710448 |
| L-Histidine, USP | Sigma | Cat # H-6034 Lot 31K0891 |
| Sodium Ascorbate, USP | Spectrum | Cat # S1349, Lot # RM0398 |
| Sodium Pyruvate | Fluka Chemika | Cat # 15990, Lot 431657/1, 34902013 |
| Fetal Bovine Serum (unirradiated) | JRH Biosciences | Cat # 12103-500M, Lot # 1H0586 |
| Fetal Bovine Serum (unirradiated) | Gibco | Cat # 26140-079, Lot # 1158690 |
| Fetal Bovine Serum (unirradiated) | Hyclone | Cat # SH30070.03.ir, Lot #AND18475 |
| Fetal Bovine Serum (unirradiated) | Hyclone | Cat # SH30071.03, Lot #ANB18202 |
| Fetal Bovine Serum (unirradiated) | Sigma | Cat # F2442, Lot # 62K8400 |

Equipment

| Item | Manufacturer | Model Number/Description |
|---|---|---|
| pH Meter | Fisher | Accumet AR50 |
| Analytical Balance | Mettler Toledo | AG245 |
| Water baths | | |
| Serum or plasma | | |
| Culture hood | | |
| Autoclave | | |

General

| Item | Item |
|---|---|
| 540 Wheaton Serum Vials, 3 mL | 10 mL Eppendorf Pipette tips |
| | (sterile) |
| 540 rubber Stelmi Stoppers | Spatulas, weigh boats |
| 540 Wheaton Aluminum Caps | Media bottle (sterile) |
| Pipettes | Metal trays |
| Repeat pipettor | Liquid Nitrogen |
| 50 mL Falcon centrifuge tubes | Filtration system (50 ml) |

### Sample Preparation

Solutions

500 mM Sodium Pyruvate, 100 mL

Dissolved 5.50 g Sodium Pyruvate in approximately 90 mL ddH₂O.

Brought the final volume to 100 mL with ddH₂O

Filtered using a 0.22 μm filter 500 mM L-Histidine. 60 mL

Dissolved 4.66 g of L-Histidine in approximately 90 mL ddH₂O.

Adjusted the pH of the solution to about pH 6.0 using HCl and NaOH as needed.

Brought the final volume to 100 mL with ddH2O

Filter using a 0.22 μm filter

2.0M Sodium Ascorbate. 20 mL

Dissolved 7.92 g of sodium ascorbate in approximately 10 mL ddH2O.

Brought the final volume to 20 mL with ddH₂O

Filtered using a 0.22 um filter

Serum Preparation

1. Thawed 250 mL of fetal bovine serum (FBS) in a room temperature water bath with gentle agitation.

2. Divided each serum into three aliquots of about 82 mL

3. To one aliquot, added 9.56 mL ddH₂O, making the unformulated control that compensates for dilution of the formulated serums.

4. To the second aliquot, added 9.1 mL 500 mM sodium pyruvate and 0.46 mL ddH₂O.

5. To the third aliquot, added 9.1 mL 500 nM Histidine, pH 6.0, and 0.46 mL 2.0 M sodium ascorbate.

6. Prepared 24 vials of FBS by aliquotting 2.4 mL into 3.0 mL serum vials. Stoppered with rubber stoppers.

7. Placed the remaining ˜30 mL in a 50 mL conical tube for complement heat inactivation.

Preparation of the Fetal Bovine Serum

| | | 500 mM | 500 mM | 2.0 M Sodium | | Total |
|---|---|---|---|---|---|---|
| | FBS | Pyruvate | Histidine | Ascorbate | ddH₂O | Volume |
| Formulation | (mL) | (mL) | (mL) | (mL) | (mL) | (mL) |
| No Excipients | 82 | — | — | — | 0.46 | 91.6 |
| 50 mM Pyruvate | 82 | 9.1 | — | — | 0.46 | 91.6 |
| 50 mM His. + 10 mM Asc. | 82 | — | 9.1 | 0.46 | — | 91.6 |

Complement Inactivation

1. Set the temperature of the water bath to 60° C.

2. Prepared a “blank” tube by placing 30 mL of water at room temperature into a 50 mL tube.

3. Placed a thermocouple in the tube to monitor the temperature of the tube.

4. Put the blank and the tubes of FBS into the water bath, set at 60° C.

5. Monitored the temperature of the thermocoupled blank. When it reached 56° C., began timing for 30 min. and reduced the set temperature of the water bath to 57° C.

6. After 30 minutes, removed the FBS and aliquoted into vials.

Irradiation

1. All samples to be irradiated were stored at −80° C. until shipment. Samples to be irradiated at ambient temperature were stored at −80° C. until the day of shipment, when it was thawed in a room temperature water bath.

Post-Irradiation Complement Heat Inactivation

FBS was heat inactivated after irradiation as follows:

1. Set the temperature of the water bath to 56° C.

2. Prepared a “blank” tube by placing 2.4 mL of water at room temperature into a 3.0 mL vial.

3. Placed a thermocouple in the tube to monitor the temperature of the tube.

4. Brought the vials to room temperature in a room temperature water bath.

5. Put the blank and the vials of FBS into the water bath, set at 56° C.

6. Monitored the temperature of the thermocoupled blank. When it reached 56° C., began timing for 30 min.

### Methods of Analysis

Turbidity

Turbidity was measured using a DRT-100B Turbidometer from HF Scientific, Inc.

SDS-PA GE

SDS-PAGE will be performed on select samples after turbidity analysis. The gels used were Novex 1.5 mm, 10 well, 4-20% gradient gels. Staining was done using Coomassie Blue stain.

Differential Scanning Calorimetry

Differential Scanning Calorimetry, if performed, was carried out after turbidity analysis using standard instrumentation and techniques.

HPLC Analysis

HPLC Analysis was performed using standard instrumentation and techniques.

Exemplary conditions for carrying out the above protocol include the following:

TABLE 1
Hyclone Defined Serum: No Excipients

| | | Heat | Gamma | Gamma | |
|---|---|---|---|---|---|
| Serum | Formulation | Inactivation | Dose (kGy) | Temperature | # of Vials |
| Hyclone Defined | No Excipients | None | 40 | Ambient | 2 |
| Hyclone Defined | No Excipients | Before | 40 | Ambient | 1 |
| Hyclone Defined | No Excipients | After | 40 | Ambient | 1 |
| Hyclone Defined | No Excipients | None | 40 | −10 | 2 |
| Hyclone Defined | No Excipients | Before | 40 | −10 | 1 |
| Hyclone Defined | No Excipients | After | 40 | −10 | 1 |
| Hyclone Defined | No Excipients | None | 40 | −20 | 2 |
| Hyclone Defined | No Excipients | Before | 40 | −20 | 1 |
| Hyclone Defined | No Excipients | After | 40 | −20 | 1 |
| Hyclone Defined | No Excipients | None | 40 | −30 | 2 |
| Hyclone Defined | No Excipients | Before | 40 | −30 | 1 |
| Hyclone Defined | No Excipients | After | 40 | −30 | 1 |
| Hyclone Defined | No Excipients | None | 40 | −40 | 2 |
| Hyclone Defined | No Excipients | Before | 40 | −40 | 1 |
| Hyclone Defined | No Excipients | After | 40 | −40 | 1 |
| Hyclone Defined | No Excipients | None | 40 | −45 | 2 |
| Hyclone Defined | No Excipients | Before | 40 | −45 | 1 |
| Hyclone Defined | No Excipients | After | 40 | −45 | 1 |
| Hyclone Defined | No Excipients | None | 40 | −50 | 2 |
| Hyclone Defined | No Excipients | Before | 40 | −50 | 1 |
| Hyclone Defined | No Excipients | After | 40 | −50 | 1 |
| Hyclone Defined | No Excipients | None | 40 | −55 | 2 |
| Hyclone Defined | No Excipients | Before | 40 | −55 | 1 |
| Hyclone Defined | No Excipients | After | 40 | −55 | 1 |
| Hyclone Defined | No Excipients | None | 40 | Dry Ice | 2 |
| Hyclone Defined | No Excipients | Before | 40 | Dry Ice | 1 |
| Hyclone Defined | No Excipients | After | 40 | Dry Ice | 1 |

TABLE 2
Hyclone Defined Serum: Pyruvate

| | | Heat | Gamma | Gamma | |
|---|---|---|---|---|---|
| Serum | Formulation | Inactivation | Dose (kGy) | Temperature | # of Vials |
| Hyclone Defined | 50 mM Pyruvate | None | 40 | Ambient | 2 |
| Hyclone Defined | 50 mM Pyruvate | Before | 40 | Ambient | 1 |
| Hyclone Defined | 50 mM Pyruvate | After | 40 | Ambient | 1 |
| Hyclone Defined | 50 mM Pyruvate | None | 40 | −10 | 2 |
| Hyclone Defined | 50 mM Pyruvare | Before | 40 | −10 | 1 |
| Hyclone Defined | 50 mM Pyruvate | After | 40 | −10 | 1 |
| Hyclone Defined | 50 mM Pyruvate | None | 40 | −20 | 2 |
| Hyclone Defined | 50 mM Pyruvate | Before | 40 | −20 | 1 |
| Hyclone Defined | 50 mM Pyruvate | After | 40 | −20 | 1 |
| Hyclone Defined | 50 mM Pyruvate | None | 40 | −30 | 2 |
| Hyclone Defined | 50 mM Pyruvate | Before | 40 | −30 | 1 |
| Hyclone Defined | 50 mM Pyruvate | After | 40 | −30 | 1 |
| Hyclone Defined | 50 mM Pyruvate | None | 40 | −40 | 2 |
| Hyclone Defined | 50 mM Pyruvate | Before | 40 | −40 | 1 |
| Hyclone Defined | 50 mM Pyruvate | After | 40 | −40 | 1 |
| Hyclone Defined | 50 mM Pyruvate | None | 40 | −45 | 2 |
| Hyclone Defined | 50 mM Pyruvate | Before | 40 | −45 | 1 |
| Hyclone Defined | 50 mM Pyruvate | After | 40 | −45 | 1 |
| Hyclone Defined | 50 mM Pyruvate | None | 40 | −50 | 2 |
| Hyclone Defined | 50 mM Pyruvate | Before | 40 | −50 | 1 |
| Hyclone Defined | 50 mM Pyruvate | After | 40 | −50 | 1 |
| Hyclone Defined | 50 mM Pyruvate | None | 40 | −55 | 2 |
| Hyclone Defined | 50 mM Pyruvate | Before | 40 | −55 | 1 |
| Hyclone Defined | 50 mM Pyruvate | After | 40 | −55 | 1 |
| Hyclone Defined | 50 mM Pyruvate | None | 40 | Dry Ice | 2 |
| Hyclone Defined | 50 mM Pyruvate | Before | 40 | Dry Ice | 1 |
| Hyclone Defined | 50 mM Pyruvate | After | 40 | Dry Ice | 1 |

TABLE 3
Hyclone Defined Serum: Histidine and Ascorbate

| | | Heat | Gamma | Gamma | |
|---|---|---|---|---|---|
| Serum | Formulation | Inactivation | Dose (kGy) | Temperature | # of Vials |
| Hyclone Defined | 50 mM His + 10 mM Asc | None | 40 | Ambient | 2 |
| Hyclone Defined | 50 mM His + 10 mM Asc | Before | 40 | Ambient | 1 |
| Hyclone Defined | 50 mM His + 10 mM Asc | After | 40 | Ambient | 1 |
| Hyclone Defined | 50 mM His + 10 mM Asc | None | 40 | −10 | 2 |
| Hyclone Defined | 50 mM His + 10 mM Asc | Before | 40 | −10 | 1 |
| Hyclone Defined | 50 mM His + 10 mM Asc | After | 40 | −10 | 1 |
| Hyclone Defined | 50 mM His + 10 mM Asc | None | 40 | −20 | 2 |
| Hyclone Defined | 50 mM His + 10 mM Asc | Before | 40 | −20 | 1 |
| Hyclone Defined | 50 mM His + 10 mM Asc | After | 40 | −20 | 1 |
| Hyclone Defined | 50 mM His + 10 mM Asc | None | 40 | −30 | 2 |
| Hyclone Defined | 50 mM His + 10 mM Asc | Before | 40 | −30 | 1 |
| Hyclone Defined | 50 mM His + 10 mM Asc | After | 40 | −30 | 1 |
| Hyclone Defined | 50 mM His + 10 mM Asc | None | 40 | −40 | 2 |
| Hyclone Defined | 50 mM His + 10 mM Asc | Before | 40 | −40 | 1 |
| Hyclone Defined | 50 mM His + 10 mM Asc | After | 40 | −40 | 1 |
| Hyclone Defined | 50 mM His + 10 mM Asc | None | 40 | −45 | 2 |
| Hyclone Defined | 50 mM His + 10 mM Asc | Before | 40 | −45 | 1 |
| Hyclone Defined | 50 mM His + 10 mM Asc | After | 40 | −45 | 1 |
| Hyclone Defined | 50 mM His + 10 mM Asc | None | 40 | −50 | 2 |
| Hyclone Defined | 50 mM His + 10 mM Asc | Before | 40 | −50 | 1 |
| Hyclone Defined | 50 mM His + 10 mM Asc | After | 40 | −50 | 1 |
| Hyclone Defined | 50 mM His + 10 mM Asc | None | 40 | −55 | 2 |
| Hyclone Defined | 50 mM His + 10 mM Asc | Before | 40 | −55 | 1 |
| Hyclone Defined | 50 mM His + 10 mM Asc | After | 40 | −55 | 1 |
| Hyclone Defined | 50 mM His + 10 mM Asc | None | 40 | Dry Ice | 2 |
| Hyclone Defined | 50 mM His + 10 mM Ase | Before | 40 | Dry Ice | 1 |
| Hyclone Defined | 50 mM His + 10 mM Asc | After | 40 | Dry Ice | 1 |

TABLE 4
Hyclone Characterized Serum: No Excipients

| | | Heat | Gamma | Gamma | |
|---|---|---|---|---|---|
| Serum | Formulation | Inactivation | Dose (kGy) | Temperature | # of Vials |
| Hyclone Char. | No Excipients | None | 40 | Ambient | 1 |
| Hyclone Char. | No Excipients | Before | 40 | Ambient | 1 |
| Hyclone Char. | No Excipients | After | 40 | Ambient | 1 |
| Hyclone Char. | No Excipients | None | 40 | −10 | 1 |
| Hyclone Char. | No Excipients | Before | 40 | −10 | 1 |
| Hyclone Char. | No Excipients | After | 40 | −10 | 1 |
| Hyclone Char. | No Excipients | None | 40 | −20 | 1 |
| Hyclone Char. | No Excipients | Before | 40 | −20 | 1 |
| Hyclone Char. | No Excipients | After | 40 | −20 | 1 |
| Hyclone Char. | No Excipients | None | 40 | −30 | 1 |
| Hyclone Char. | No Excipients | Before | 40 | −30 | 1 |
| Hyclone Char. | No Excipients | After | 40 | −30 | 1 |
| Hyclone Char. | No Excipients | None | 40 | −40 | 1 |
| Hyclone Char. | No Excipients | Before | 40 | −40 | 1 |
| Hyclone Char. | No Excipients | After | 40 | −40 | 1 |
| Hyclone Char. | No Excipients | None | 40 | −45 | 1 |
| Hyclone Char. | No Excipients | Before | 40 | −45 | 1 |
| Hyclone Char. | No Excipients | After | 40 | −45 | 1 |
| Hyclone Char. | No Excipients | None | 40 | −50 | 1 |
| Hyclone Char. | No Excipients | Before | 40 | −50 | 1 |
| Hyclone Char. | No Excipients | After | 40 | −50 | 1 |
| Hyclone Char. | No Excipients | None | 40 | −55 | 1 |
| Hyclone Char. | No Excipients | Before | 40 | −55 | 1 |
| Hyclone Char. | No Excipients | After | 40 | −55 | 1 |
| Hyclone Char. | No Excipients | None | 40 | Dry Ice | 1 |
| Hyclone Char. | No Excipients | Before | 40 | Dry Ice | 1 |
| Hyclone Char. | No Excipients | After | 40 | Dry Ice | 1 |

TABLE 5
Invitrogen Serum: No Excipients

| | | | Gamma | | |
|---|---|---|---|---|---|
| | | Heat | Dose | Gamma | # of |
| Serum | Formulation | Inactivation | (kGy) | Temperature | Vials |
| Invitrogen | No Excipients | None | 40 | Ambient | 2 |
| Invitrogen | No Excipients | Before | 40 | Ambient | 1 |
| Invitrogen | No Excipients | After | 40 | Ambient | 1 |
| Invitrogen | No Excipients | None | 40 | −10 | 2 |
| Invitrogen | No Excipients | Before | 40 | −10 | 1 |
| Invitrogen | No Excipients | After | 40 | −10 | 1 |
| Invitrogen | No Excipients | None | 40 | −20 | 2 |
| Invitrogen | No Excipients | Before | 40 | −20 | 1 |
| Invitrogen | No Excipients | After | 40 | −20 | 1 |
| Invitrogen | No Excipients | None | 40 | −30 | 2 |
| Invitrogen | No Excipients | Before | 40 | −30 | 1 |
| Invitrogen | No Excipients | After | 40 | −30 | 1 |
| Invitrogen | No Excipients | None | 40 | −40 | 2 |
| Invitrogen | No Excipients | Before | 40 | −40 | 1 |
| Invitrogen | No Excipients | After | 40 | −40 | 1 |
| Invitrogen | No Excipients | None | 40 | −45 | 2 |
| Invitrogen | No Excipients | Before | 40 | −45 | 1 |
| Invitrogen | No Excipients | After | 40 | −45 | 1 |
| Invitrogen | No Excipients | None | 40 | −50 | 2 |
| Invitrogen | No Excipients | Before | 40 | −50 | 1 |
| Invitrogen | No Excipients | After | 40 | −50 | 1 |
| Invitrogen | No Excipients | None | 40 | −55 | 2 |
| Invitrogen | No Excipients | Before | 40 | −55 | 1 |
| Invitrogen | No Excipients | After | 40 | −55 | 1 |
| Invitrogen | No Excipients | None | 40 | Dry Ice | 2 |
| Invitrogen | No Excipients | Before | 40 | Dry Ice | 1 |
| Invitrogen | No Excipients | After | 40 | Dry Ice | 1 |

TABLE 6
Invitrogen Serum: Pyruvate

| | | Heat | Gamma | | # |
|---|---|---|---|---|---|
| | | Inacti- | Dose | Gamma | of |
| Serum | Formulation | vation | (kGy) | Temperature | Vials |
| Invitrogen | 50 mM Pyruvate | None | 40 | Ambient | 2 |
| Invitrogen | 50 mM Pyruvate | Before | 40 | Ambient | 1 |
| Invitrogen | 50 mM Pyruvate | After | 40 | Ambient | 1 |
| Invitrogen | 50 mM Pyruvate | None | 40 | −10 | 2 |
| Invitrogen | 50 mM Pyruvate | Before | 40 | −10 | 1 |
| Invitrogen | 50 mM Pyruvate | After | 40 | −10 | 1 |
| Invitrogen | 50 mM Pyruvate | None | 40 | −20 | 2 |
| Invitrogen | 50 mM Pyruvate | Before | 40 | −20 | 1 |
| Invitrogen | 50 mM Pyruvate | After | 40 | −20 | 1 |
| Invitrogen | 50 mM Pyruvate | None | 40 | −30 | 2 |
| Invitrogen | 50 mM Pyruvate | Before | 40 | −30 | 1 |
| Invitrogen | 50 mM Pyruvate | After | 40 | −30 | 1 |
| Invitrogen | 50 mM Pyruvate | None | 40 | −40 | 2 |
| Invitrogen | 50 mM Pyruvate | Before | 40 | −40 | 1 |
| Invitrogen | 50 mM Pyruvate | After | 40 | −40 | 1 |
| Invitrogen | 50 mM Pyruvate | None | 40 | −45 | 2 |
| Invitrogen | 50 mM Pyruvate | Before | 40 | −45 | 1 |
| Invitrogen | 50 mM Pyruvate | After | 40 | −45 | 1 |
| Invitrogen | 50 mM Pyruvate | None | 40 | −50 | 2 |
| Invitrogen | 50 mM Pyruvate | Before | 40 | −50 | 1 |
| Invitrogen | 50 mM Pyruvate | After | 40 | −50 | 1 |
| Invitrogen | 50 mM Pyruvate | None | 40 | −55 | 2 |
| Invitrogen | 50 mM Pyruvate | Before | 40 | −55 | 1 |
| Invitrogen | 50 mM Pyruvate | After | 40 | −55 | 1 |
| Invitrogen | 50 mM Pyruvate | None | 40 | Dry Ice | 2 |
| Invitrogen | 50 mM Pyruvate | Before | 40 | Dry Ice | 1 |
| Invitrogen | 50 mM Pyruvate | After | 40 | Dry Ice | 1 |

TABLE 7
Invitrogen Serum: Histidine and Ascorbate

| | | Heat | Gamma | Gamma | |
|---|---|---|---|---|---|
| Serum | Formulation | Inactivation | Dose (kGy) | Temperature | # of Vials |
| Invitrogen | 50 mM His + 10 mM Asc | None | 40 | Ambient | 2 |
| Invitrogen | 50 mM His + 10 mM Asc | Before | 40 | Ambient | 1 |
| Invitrogen | 50 mM His + 10 mM Asc | After | 40 | Ambient | 1 |
| Invitrogen | 50 mM His + 10 mM Asc | None | 40 | −10 | 2 |
| Invitrogen | 50 mM His + 10 mM Asc | Before | 40 | −10 | 1 |
| Invitrogen | 50 mM His + 10 mM Asc | After | 40 | −10 | 1 |
| Invitrogen | 50 mM His + 10 mM Asc | None | 40 | −20 | 2 |
| Invitrogen | 50 mM His + 10 mM Asc | Before | 40 | −20 | 1 |
| Invitrogen | 50 mM His + 10 mM Asc | After | 40 | −20 | 1 |
| Invitrogen | 50 mM His + 10 mM Asc | None | 40 | −30 | 2 |
| Invitrogen | 50 mM His + 10 mM Asc | Before | 40 | −30 | 1 |
| Invitrogen | 50 mM His + 10 mM Asc | After | 40 | −30 | 1 |
| Invitrogen | 50 mM His + 10 mM Asc | None | 40 | −40 | 2 |
| Invitrogen | 50 mM His + 10 mM Asc | Before | 40 | −40 | 1 |
| Invitrogen | 50 mM His + 10 mM Asc | After | 40 | −40 | 1 |
| Invitrogen | 50 mM His + 10 mM Asc | None | 40 | −45 | 2 |
| Invitrogen | 50 mM His + 10 mM Asc | Before | 40 | −45 | 1 |
| Invitrogen | 50 mM His + 10 mM Asc | After | 40 | −45 | 1 |
| Invitrogen | 50 mM His + 10 mM Asc | None | 40 | −50 | 2 |
| Invitrogen | 50 mM His + 10 mM Asc | Before | 40 | −50 | 1 |
| Invitrogen | 50 mM His + 10 mM Asc | After | 40 | −50 | 1 |
| Invitrogen | 50 mM His + 10 mM Asc | None | 40 | −55 | 2 |
| Invitrogen | 50 mM His + 10 mM Asc | Before | 40 | −55 | 1 |
| Invitrogen | 50 mM His + 10 mM Asc | After | 40 | −55 | 1 |
| Invitrogen | 50 mM His + 10 mM Asc | None | 40 | Dry Ice | 2 |
| Invitrogen | 50 mM His + 10 mM Asc | Before | 40 | Dry Ice | 1 |
| Invitrogen | 50 mM His + 10 mM Asc | After | 40 | Dry Ice | 1 |

TABLE 8
JRH Serum: No Excipients

| | | | Gamma | | |
|---|---|---|---|---|---|
| | | Heat | Dose | Gamma | # of |
| Serum | Formulation | Inactivation | (kGy) | Temperature | Vials |
| JRH | No Excipients | None | 40 | Ambient | 2 |
| JRH | No Excipients | Before | 40 | Ambient | 1 |
| JRH | No Excipients | After | 40 | Ambient | 1 |
| JRH | No Excipients | None | 40 | −10 | 2 |
| JRH | No Excipients | Before | 40 | −10 | 1 |
| JRH | No Excipients | After | 40 | −10 | 1 |
| JRH | No Excipients | None | 40 | −20 | 2 |
| JRH | No Excipients | Before | 40 | −20 | 1 |
| JRH | No Excipients | After | 40 | −20 | 1 |
| JRH | No Excipients | None | 40 | −30 | 2 |
| JRH | No Excipients | Before | 40 | −30 | 1 |
| JRH | No Excipients | After | 40 | −30 | 1 |
| JRH | No Excipients | None | 40 | −40 | 2 |
| JRH | No Excipients | Before | 40 | −40 | 1 |
| JRH | No Excipients | After | 40 | −40 | 1 |
| JRH | No Excipients | None | 40 | −45 | 2 |
| JRH | No Excipients | Before | 40 | −45 | 1 |
| JRH | No Excipients | After | 40 | −45 | 1 |
| JRH | No Excipients | None | 40 | −50 | 2 |
| JRH | No Excipients | Before | 40 | −50 | 1 |
| JRH | No Excipients | After | 40 | −50 | 1 |
| JRH | No Excipients | None | 40 | −55 | 2 |
| JRH | No Excipients | Before | 40 | −55 | 1 |
| JRH | No Excipients | After | 40 | −55 | 1 |
| JRH | No Excipients | None | 40 | Dry Ice | 2 |
| JRH | No Excipients | Before | 40 | Dry Ice | 1 |
| JRH | No Excipients | After | 40 | Dry Ice | 1 |

TABLE 9
JRH Serum: Pyruvate

| | | | Gamma | | |
|---|---|---|---|---|---|
| | | Heat | Dose | Gamma | # of |
| Serum | Formulation | Inactivation | (kGy) | Temperature | Vials |
| JRH | 50 mM Pyruvate | None | 40 | Ambient | 2 |
| JRH | 50 mM Pyruvate | Before | 40 | Ambient | 1 |
| JRH | 50 mM Pyruvate | After | 40 | Ambient | 1 |
| JRH | 50 mM Pyruvate | None | 40 | −10 | 2 |
| JRH | 50 mM Pyruvate | Before | 40 | −10 | 1 |
| JRH | 50 mM Pyruvate | After | 40 | −10 | 1 |
| JRH | 50 mM Pyruvate | None | 40 | −20 | 2 |
| JRH | 50 mM Pyruvate | Before | 40 | −20 | 1 |
| JRH | 50 mM Pyruvate | After | 40 | −20 | 1 |
| JRH | 50 mM Pyruvate | None | 40 | −30 | 2 |
| JRH | 50 mM Pyruvate | Before | 40 | −30 | 1 |
| JRH | 50 mM Pyruvate | After | 40 | −30 | 1 |
| JRH | 50 mM Pyruvate | None | 40 | −40 | 2 |
| JRH | 50 mM Pyruvate | Before | 40 | −40 | 1 |
| JRH | 50 mM Pyruvate | After | 40 | −40 | 1 |
| JRH | 50 mM Pyruvate | None | 40 | −45 | 2 |
| JRH | 50 mM Pyruvate | Before | 40 | −45 | 1 |
| JRH | 50 mM Pyruvate | After | 40 | −45 | 1 |
| JRH | 50 mM Pyruvate | None | 40 | −50 | 2 |
| JRH | 50 mM Pyruvate | Before | 40 | −50 | 1 |
| JRH | 50 mM Pyruvate | After | 40 | −50 | 1 |
| JRH | 50 mM Pyruvate | None | 40 | −55 | 2 |
| JRH | 50 mM Pyruvate | Before | 40 | −55 | 1 |
| JRH | 50 mM Pyruvate | After | 40 | −55 | 1 |
| JRH | 50 mM Pyruvate | None | 40 | Dry Ice | 2 |
| JRH | 50 mM Pyruvate | Before | 40 | Dry Ice | 1 |
| JRH | 50 mM Pyruvate | After | 40 | Dry Ice | 1 |

TABLE 10
JRH Serum: Histidine and Ascorbate

| | | Heat | Gamma | Gamma | |
|---|---|---|---|---|---|
| Serum | Formulation | Inactivation | Dose (kGy) | Temperature | # of Vials |
| JRH | 50 mM His + 10 mM Asc | None | 40 | Ambient | 2 |
| JRH | 50 mM His + 10 mM Asc | Before | 40 | Ambient | 1 |
| JRH | 50 mM His + 10 mM Asc | After | 40 | Ambient | 1 |
| JRH | 50 mM His + 10 mM Asc | None | 40 | −10 | 2 |
| JRH | 50 mM His + 10 mM Asc | Before | 40 | −10 | 1 |
| JRH | 50 mM His + 10 mM Asc | After | 40 | −10 | 1 |
| JRH | 50 mM His + 10 mM Asc | None | 40 | −20 | 2 |
| JRH | 50 mM His + 10 mM Asc | Before | 40 | −20 | 1 |
| JRH | 50 mM His + 10 mM Asc | After | 40 | −20 | 1 |
| JRH | 50 mM His + 10 mM Asc | None | 40 | −30 | 2 |
| JRH | 50 mM His + 10 mM Asc | Before | 40 | −30 | 1 |
| JRH | 50 mM His + 10 mM Asc | After | 40 | −30 | 1 |
| JRH | 50 mM His + 10 mM Asc | None | 40 | −40 | 2 |
| JRH | 50 mM His + 10 mM Asc | Before | 40 | −40 | 1 |
| JRH | 50 mM His + 10 mM Asc | After | 40 | −40 | 1 |
| JRH | 50 mM His + 10 mM Asc | None | 40 | −45 | 2 |
| JRH | 50 mM His + 10 mM Asc | Before | 40 | −45 | 1 |
| JRH | 50 mM His + 10 mM Asc | After | 40 | −45 | 1 |
| JRH | 50 mM His + 10 mM Asc | None | 40 | −50 | 2 |
| JRH | 50 mM His + 10 mM Asc | Before | 40 | −50 | 1 |
| JRH | 50 mM His + 10 mM Asc | After | 40 | −50 | 1 |
| JRH | 50 mM His + 10 mM Asc | None | 40 | −55 | 2 |
| JRH | 50 mM His + 10 mM Asc | Before | 40 | −55 | 1 |
| JRH | 50 mM His + 10 mM Asc | After | 40 | −55 | 1 |
| JRH | 50 mM His + 10 mM Asc | None | 40 | Dry Ice | 2 |
| JRH | 50 mM His + 10 mM Asc | Before | 40 | Dry Ice | 1 |
| JRH | 50 mM His + 10 mM Asc | After | 40 | Dry Ice | 1 |

TABLE 11
Sigma Serum: No Excipients

| | | | Gamma | | |
|---|---|---|---|---|---|
| | | Heat | Dose | Gamma | # |
| Serum | Formulation | Inactivation | (kGy) | Temperature | of Vials |
| Sigma | No Excipients | None | 40 | Ambient | 2 |
| Sigma | No Excipients | Before | 40 | Ambient | 1 |
| Sigma | No Excipients | After | 40 | Ambient | 1 |
| Sigma | No Excipients | None | 40 | −10 | 2 |
| Sigma | No Excipients | Before | 40 | −10 | 1 |
| Sigma | No Excipients | After | 40 | −10 | 1 |
| Sigma | No Excipients | None | 40 | −20 | 2 |
| Sigma | No Excipients | Before | 40 | −20 | 1 |
| Sigma | No Excipients | After | 40 | −20 | 1 |
| Sigma | No Excipients | None | 40 | −30 | 2 |
| Sigma | No Excipients | Before | 40 | −30 | 1 |
| Sigma | No Excipients | After | 40 | −30 | 1 |
| Sigma | No Excipients | None | 40 | −40 | 2 |
| Sigma | No Excipients | Before | 40 | −40 | 1 |
| Sigma | No Excipients | After | 40 | −40 | 1 |
| Sigma | No Excipients | None | 40 | −45 | 2 |
| Sigma | No Excipients | Before | 40 | −45 | 1 |
| Sigma | No Excipients | After | 40 | −45 | 1 |
| Sigma | No Excipients | None | 40 | −50 | 2 |
| Sigma | No Excipients | Before | 40 | −50 | 1 |
| Sigma | No Excipients | After | 40 | −50 | 1 |
| Sigma | No Excipients | None | 40 | −55 | 2 |
| Sigma | No Excipients | Before | 40 | −55 | 1 |
| Sigma | No Excipients | After | 40 | −55 | 1 |
| Sigma | No Excipients | None | 40 | Dry Ice | 2 |
| Sigma | No Excipients | Before | 40 | Dry Ice | 1 |
| Sigma | No Excipients | After | 40 | Dry Ice | 1 |

TABLE 12
Sigma Serum: Pyruvate

| | | | Gamma | | # |
|---|---|---|---|---|---|
| | | Heat | Dose | Gamma | of |
| Serum | Formulation | Inactivation | (kGy) | Temperature | Vials |
| Sigma | 50 mM Pyruvate | None | 40 | Ambient | 2 |
| Sigma | 50 mM Pyruvate | Before | 40 | Ambient | 1 |
| Sigma | 50 mM Pyruvate | After | 40 | Ambient | 1 |
| Sigma | 50 mM Pyruvate | None | 40 | −10 | 2 |
| Sigma | 50 mM Pyruvate | Before | 40 | −10 | 1 |
| Sigma | 50 mM Pyruvate | After | 40 | −10 | 1 |
| Sigma | 50 mM Pyruvate | None | 40 | −20 | 2 |
| Sigma | 50 mM Pyruvate | Before | 40 | −20 | 1 |
| Sigma | 50 mM Pyruvate | After | 40 | −20 | 1 |
| Sigma | 50 mM Pyruvate | None | 40 | −30 | 2 |
| Sigma | 50 mM Pyruvate | Before | 40 | −30 | 1 |
| Sigma | 50 mM Pyruvate | After | 40 | −30 | 1 |
| Sigma | 50 mM Pyruvate | None | 40 | −40 | 2 |
| Sigma | 50 mM Pyruvate | Before | 40 | −40 | 1 |
| Sigma | 50 mM Pyruvate | After | 40 | −40 | 1 |
| Sigma | 50 mM Pyruvate | None | 40 | −45 | 2 |
| Sigma | 50 mM Pyruvate | Before | 40 | −45 | 1 |
| Sigma | 50 mM Pyruvate | After | 40 | −45 | 1 |
| Sigma | 50 mM Pyruvate | None | 40 | −50 | 2 |
| Sigma | 50 mM Pyruvate | Before | 40 | −50 | 1 |
| Sigma | 50 mM Pyruvate | After | 40 | −50 | 1 |
| Sigma | 50 mM Pyruvate | None | 40 | −55 | 2 |
| Sigma | 50 mM Pyruvate | Before | 40 | −55 | 1 |
| Sigma | 50 mM Pyruvate | After | 40 | −55 | 1 |
| Sigma | 50 mM Pyruvate | None | 40 | Dry Ice | 2 |
| Sigma | 50 mM Pyruvate | Before | 40 | Dry Ice | 1 |
| Sigma | 50 mM Pyruvate | After | 40 | Dry Ice | 1 |

TABLE 13
Sigma Serum: Histidine and Ascorbate

| | | Heat | Gamma | Gamma | |
|---|---|---|---|---|---|
| Serum | Formulation | Inactivation | Dose (kGy) | Temperature | # of Vials |
| Sigma | 50 mM His + 10 mM Asc | None | 40 | Ambient | 2 |
| Sigma | 50 mM His + 10 mM Asc | Before | 40 | Ambient | 1 |
| Sigma | 50 mM His + 10 mM Asc | After | 40 | Ambient | 1 |
| Sigma | 50 mM His + 10 mM Asc | None | 40 | −10 | 2 |
| Sigma | 50 mM His + 10 mM Asc | Before | 40 | −10 | 1 |
| Sigma | 50 mM His + 10 mM Asc | After | 40 | −10 | 1 |
| Sigma | 50 mM His + 10 mM Asc | None | 40 | −20 | 2 |
| Sigma | 50 mM His + 10 mM Asc | Before | 40 | −20 | 1 |
| Sigma | 50 mM His + 10 mM Asc | After | 40 | −20 | 1 |
| Sigma | 50 mM His + 10 mM Asc | None | 40 | −30 | 2 |
| Sigma | 50 mM His + 10 mM Asc | Before | 40 | −30 | 1 |
| Sigma | 50 mM His + 10 mM Asc | After | 40 | −30 | 1 |
| Sigma | 50 mM His + 10 mM Asc | None | 40 | −40 | 2 |
| Sigma | 50 mM His + 10 mM Asc | Before | 40 | −40 | 1 |
| Sigma | 50 mM His + 10 mM Asc | After | 40 | −40 | 1 |
| Sigma | 50 mM His + 10 mM Asc | None | 40 | −45 | 2 |
| Sigma | 50 mM His + 10 mM Asc | Before | 40 | −45 | 1 |
| Sigma | 50 mM His + 10 mM Asc | After | 40 | −45 | 1 |
| Sigma | 50 mM His + 10 mM Asc | None | 40 | −50 | 2 |
| Sigma | 50 mM His + 10 mM Asc | Before | 40 | −50 | 1 |
| Sigma | 50 mM His + 10 mM Asc | After | 40 | −50 | 1 |
| Sigma | 50 mM His + 10 mM Asc | None | 40 | −55 | 2 |
| Sigma | 50 mM His + 10 mM Asc | Before | 40 | −55 | 1 |
| Sigma | 50 mM His + 10 mM Asc | After | 40 | −55 | 1 |
| Sigma | 50 mM His + 10 mM Asc | None | 40 | Dry Ice | 2 |
| Sigma | 50 mM His + 10 mM Asc | Before | 40 | Dry Ice | 1 |
| Sigma | 50 mM His + 10 mM Asc | After | 40 | Dry Ice | 1 |

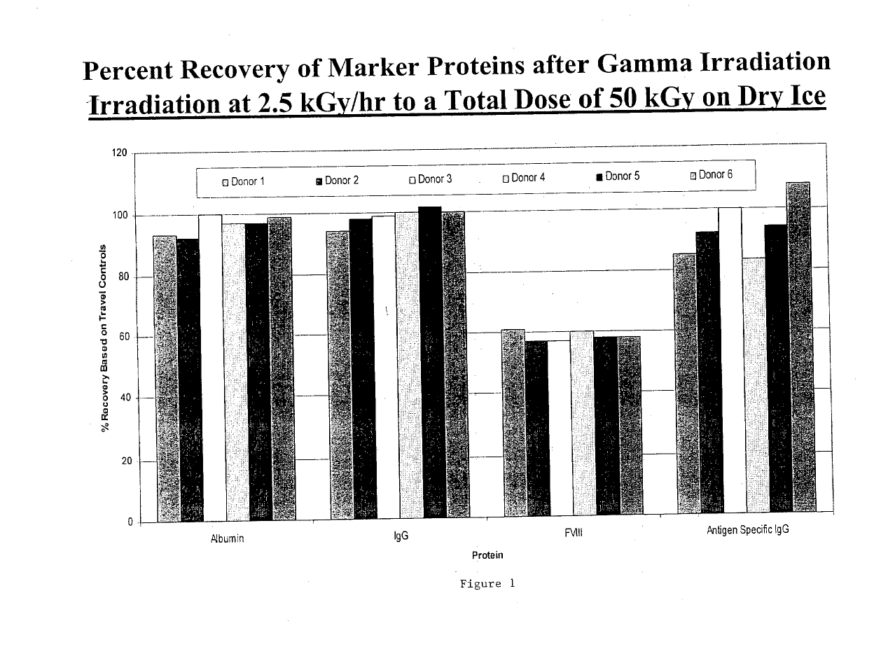

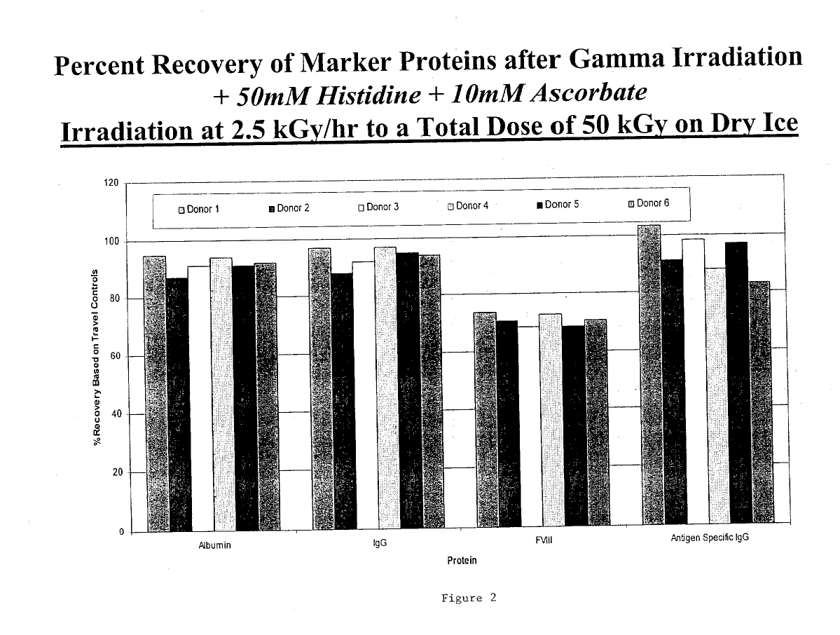

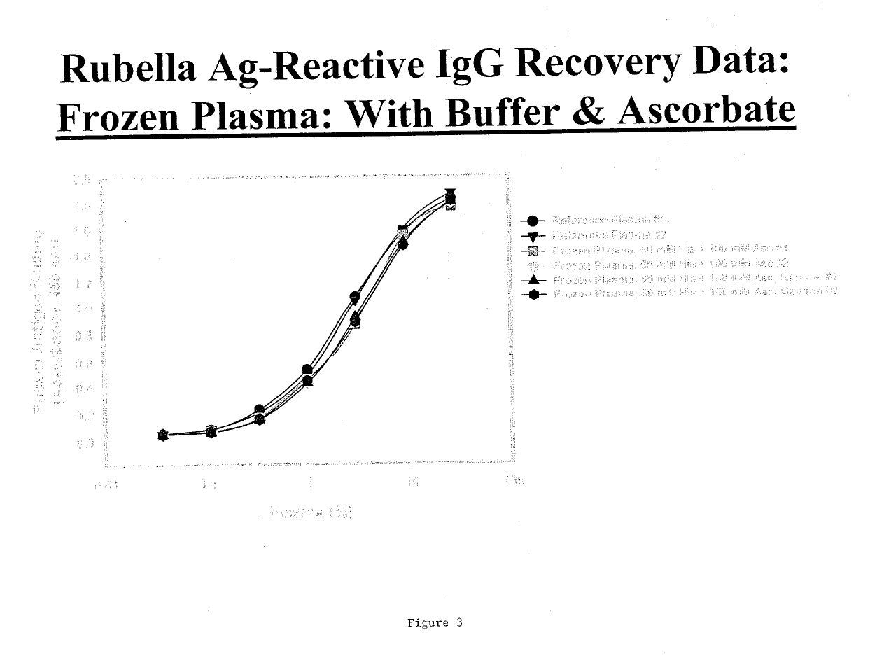

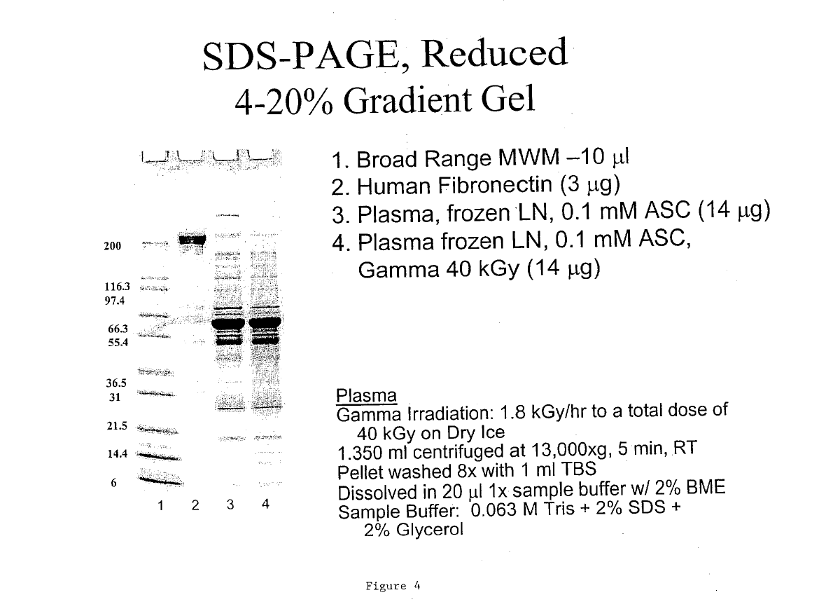

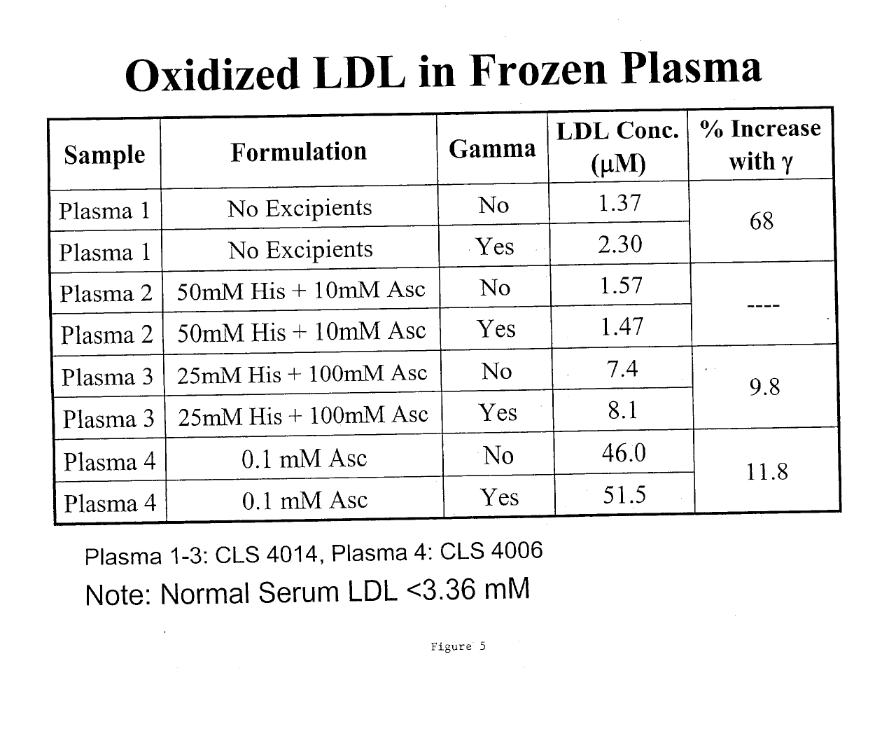

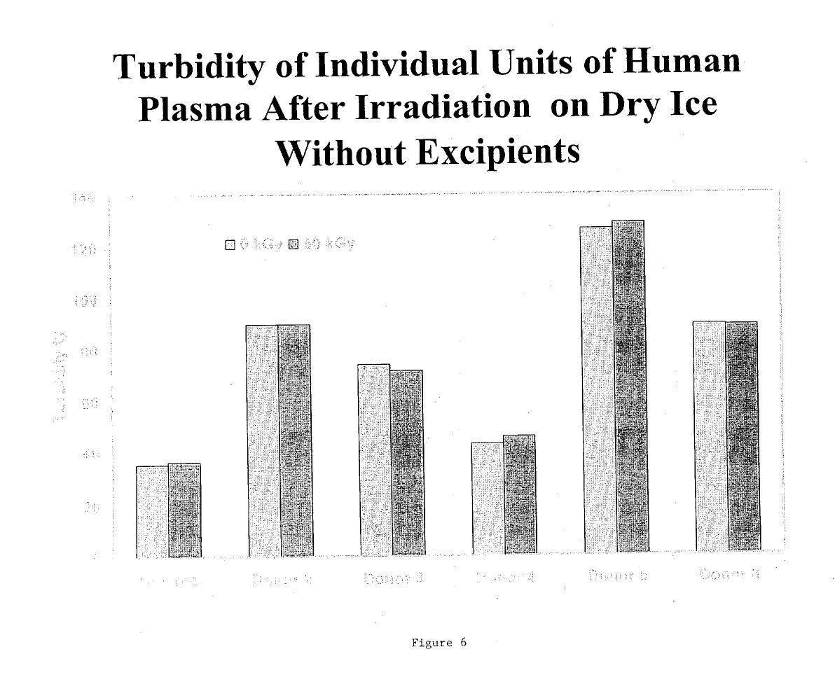

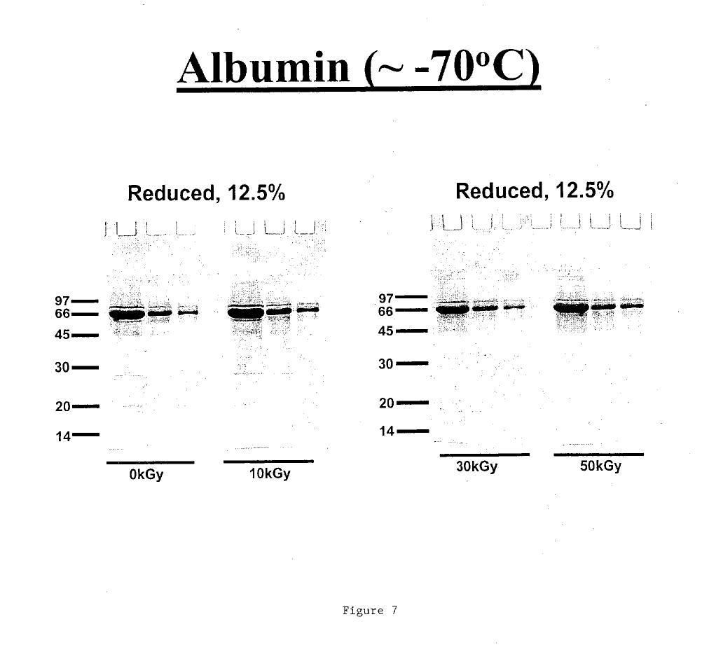

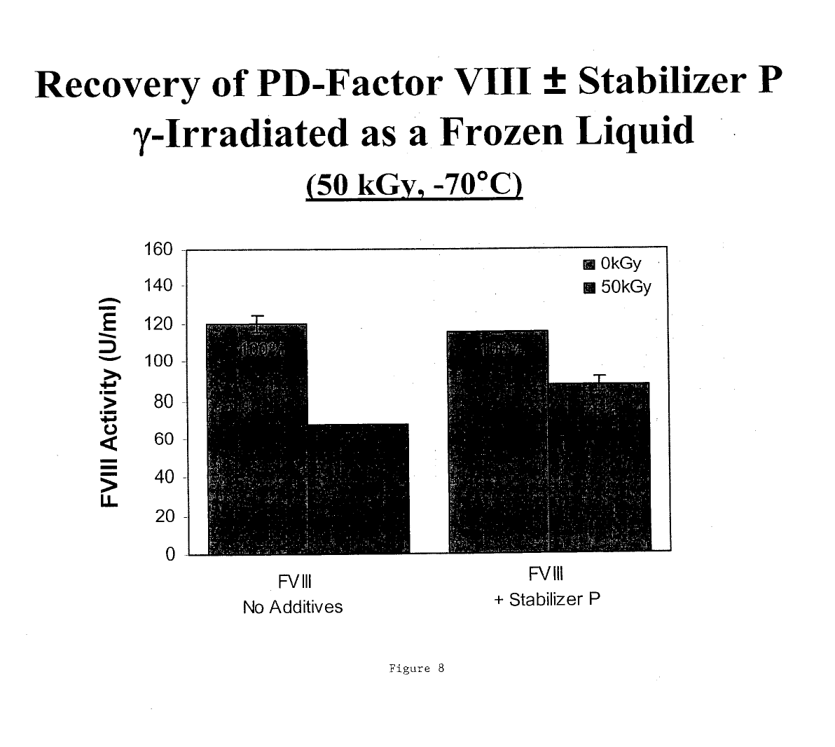

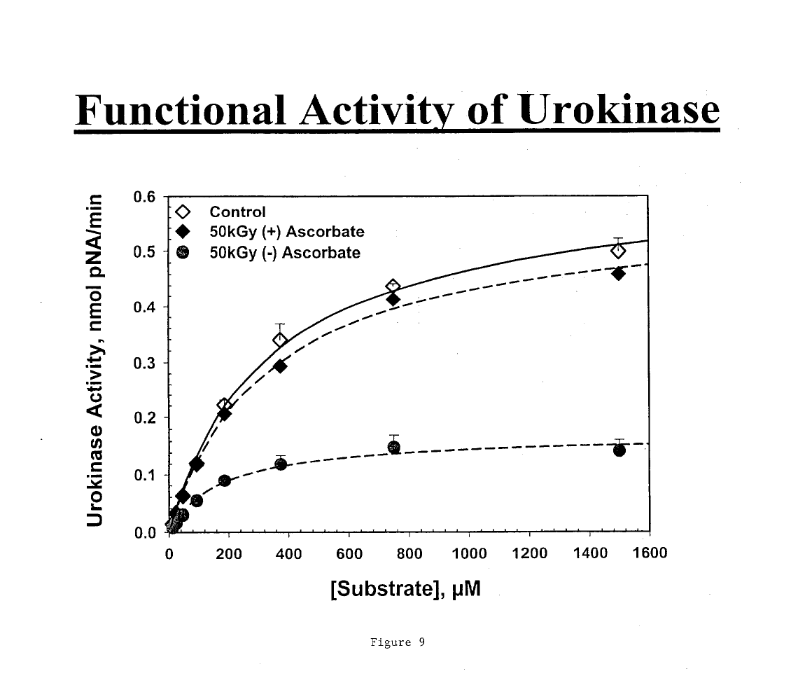

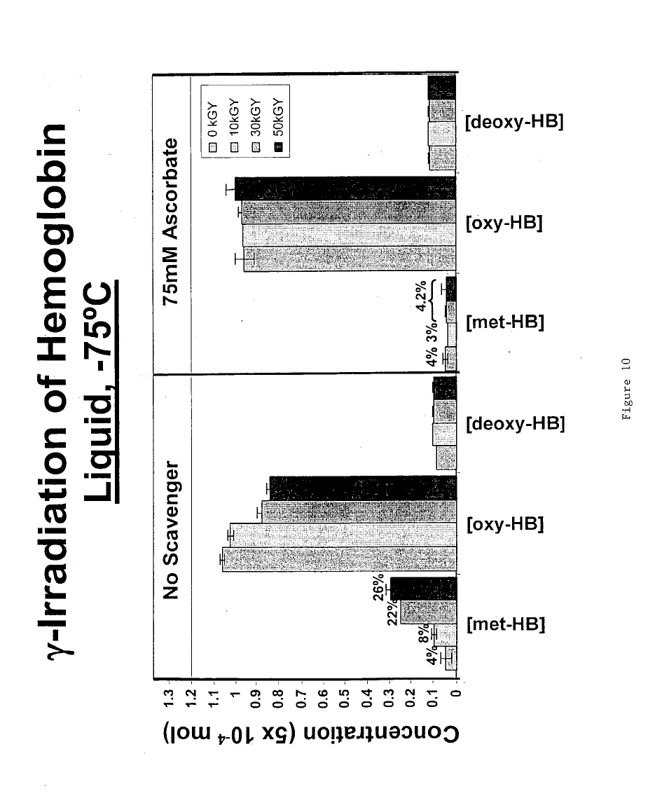

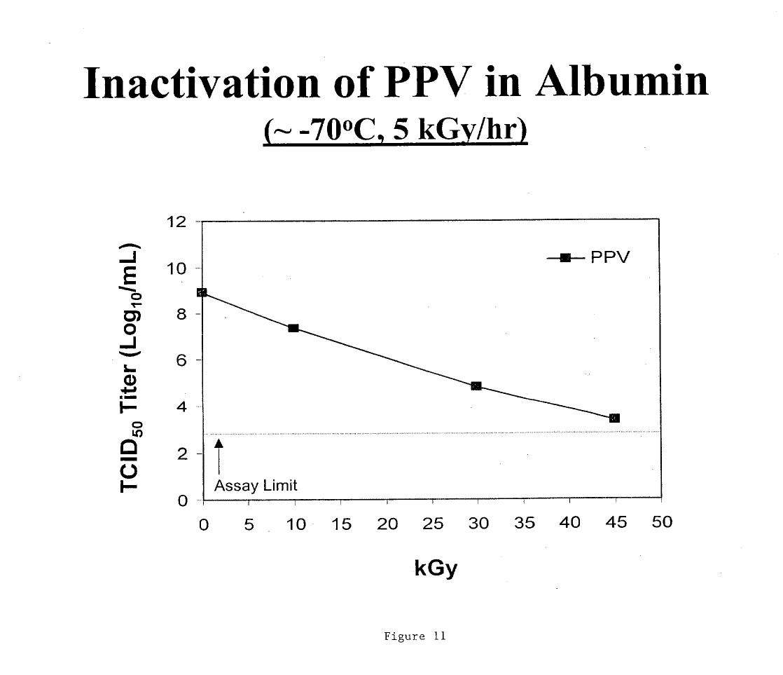

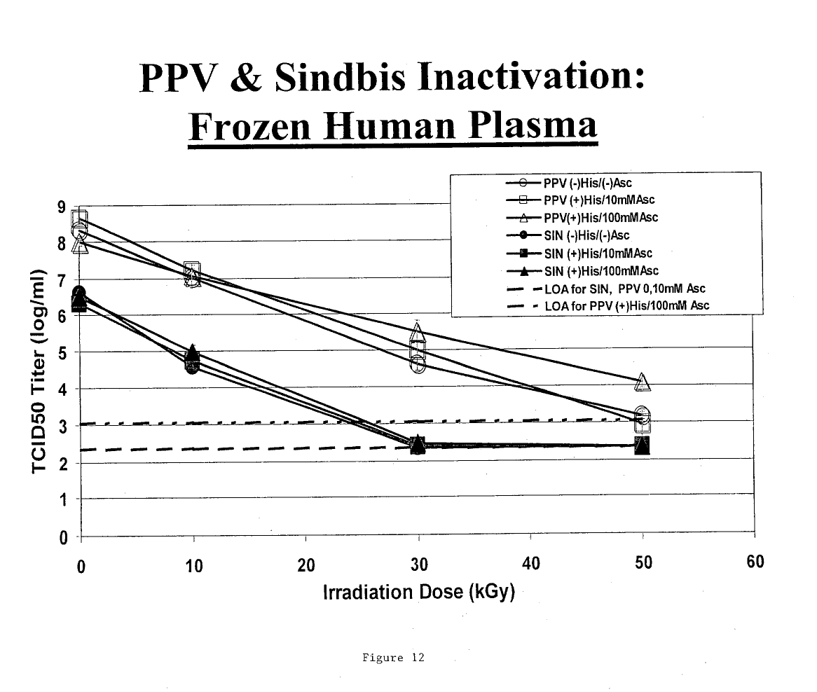

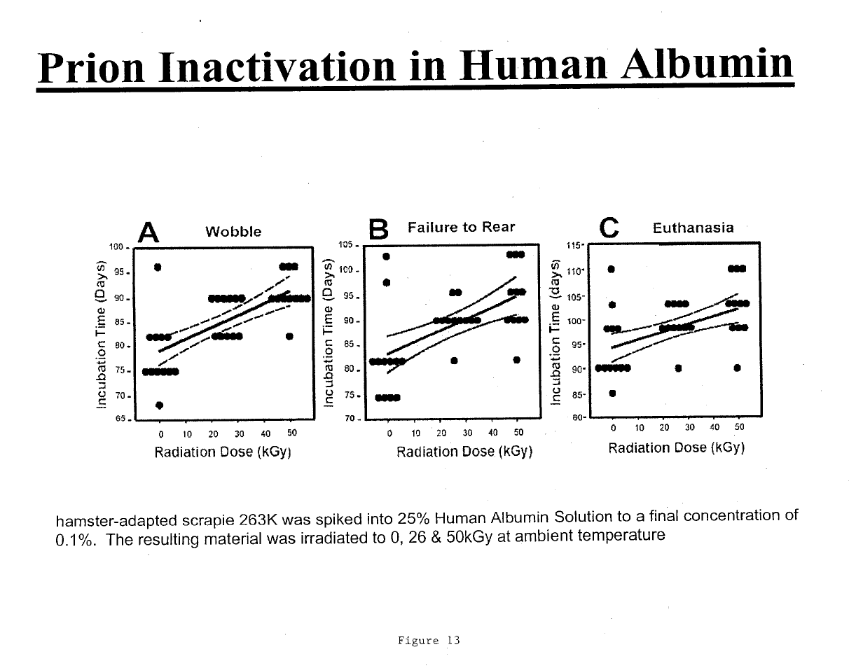

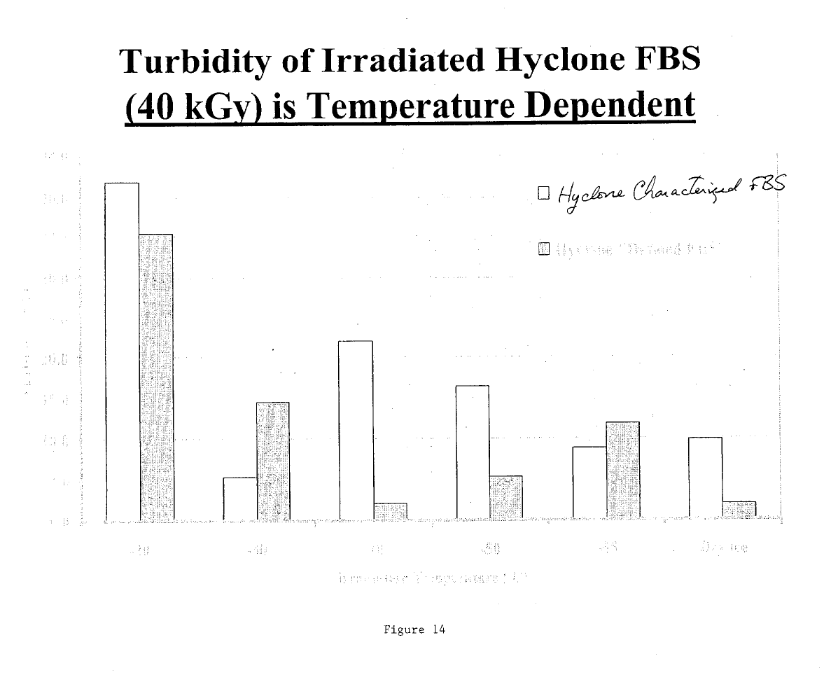

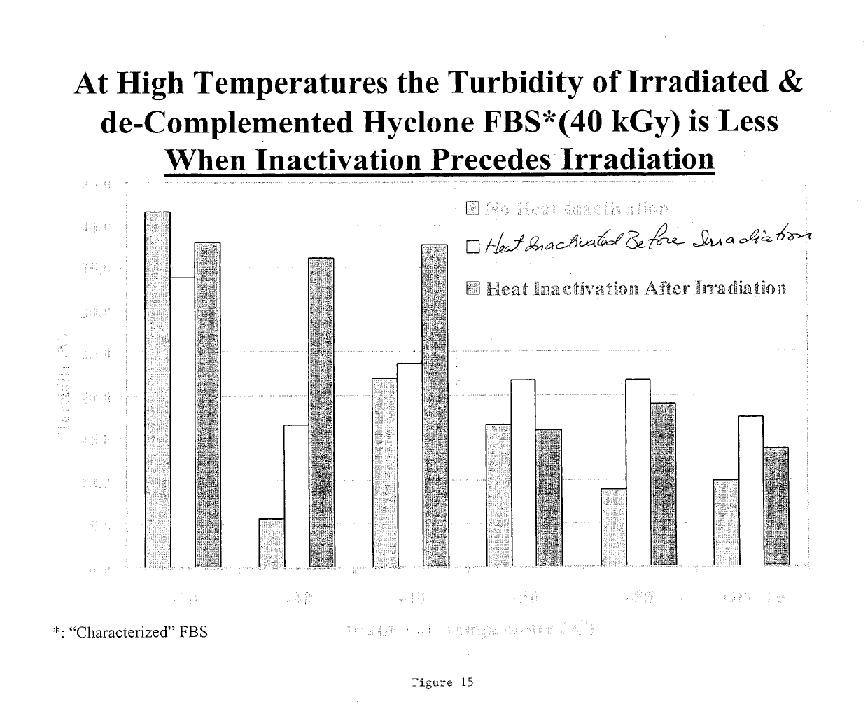

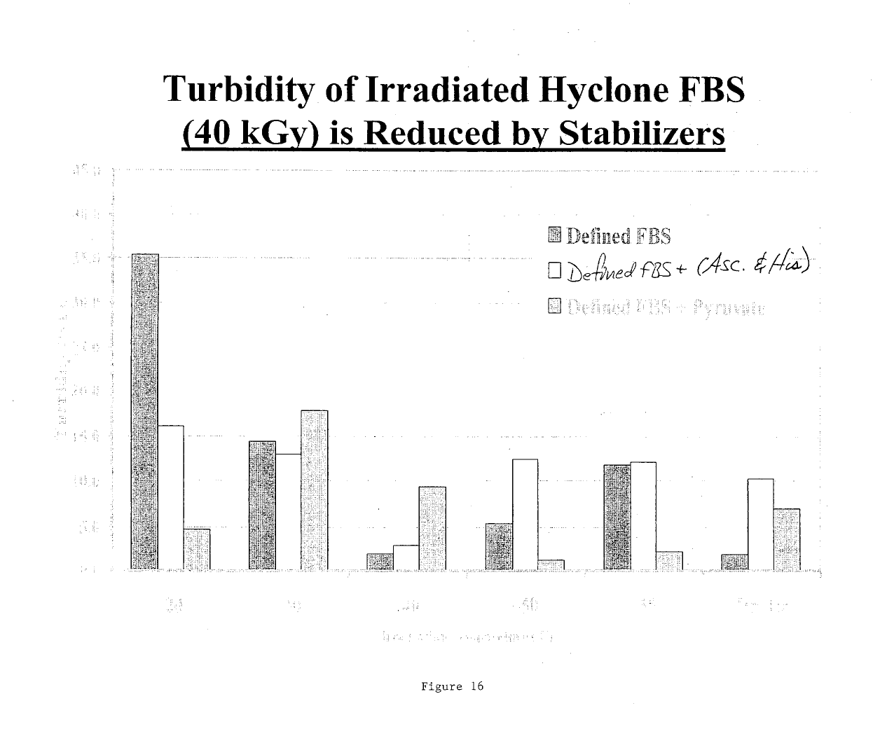

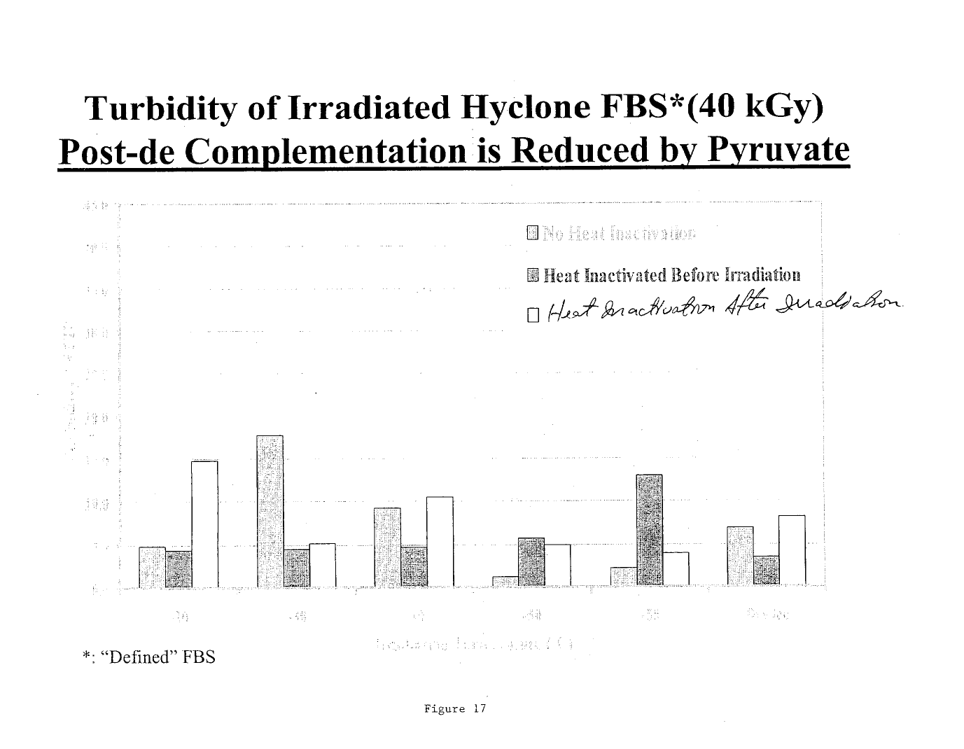

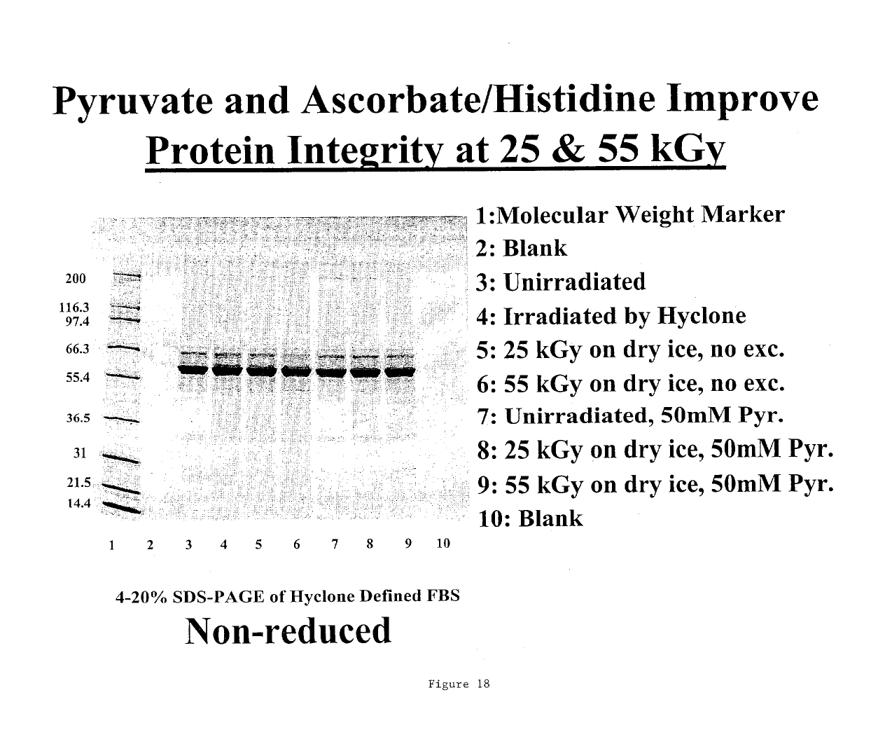

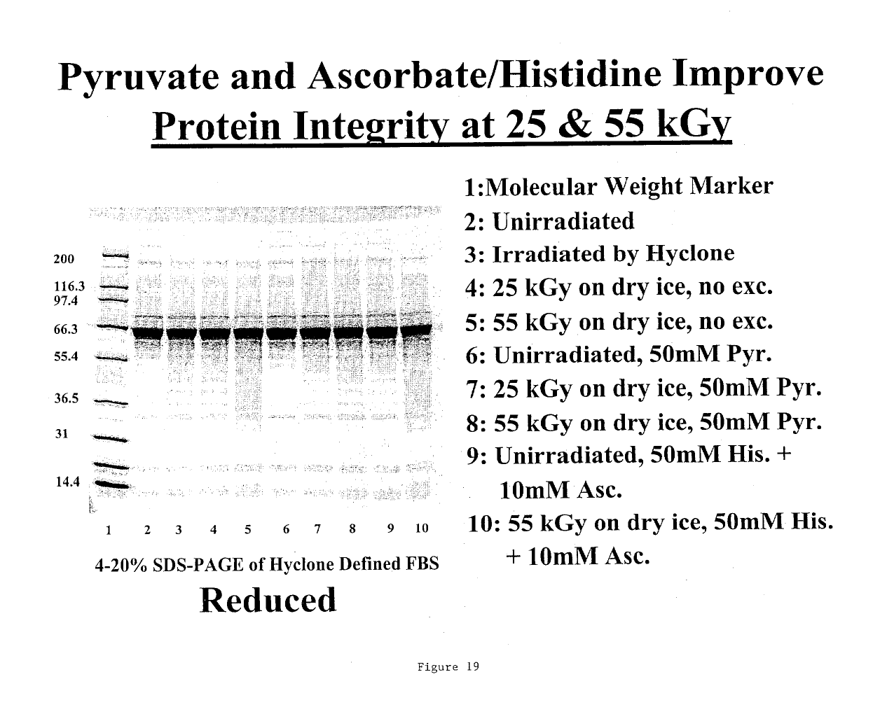

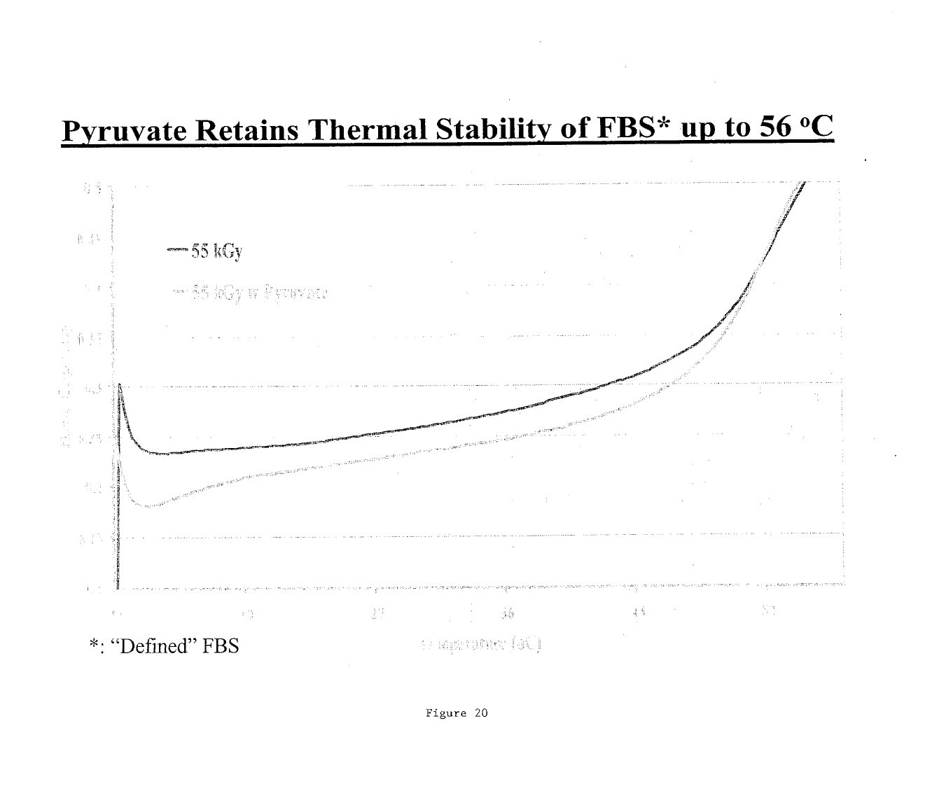

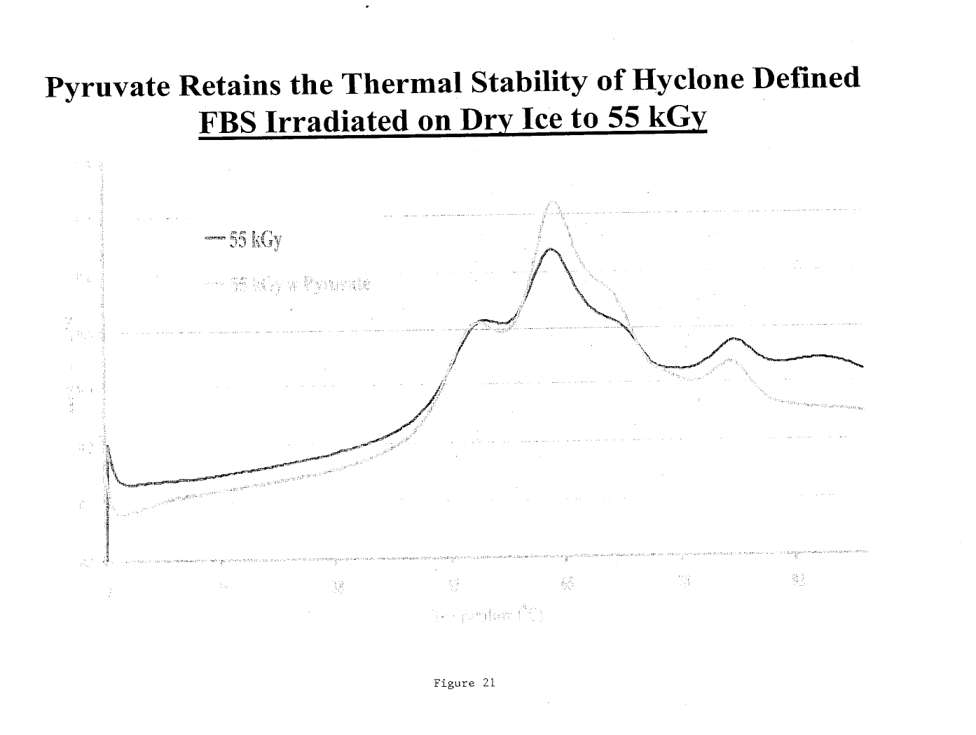

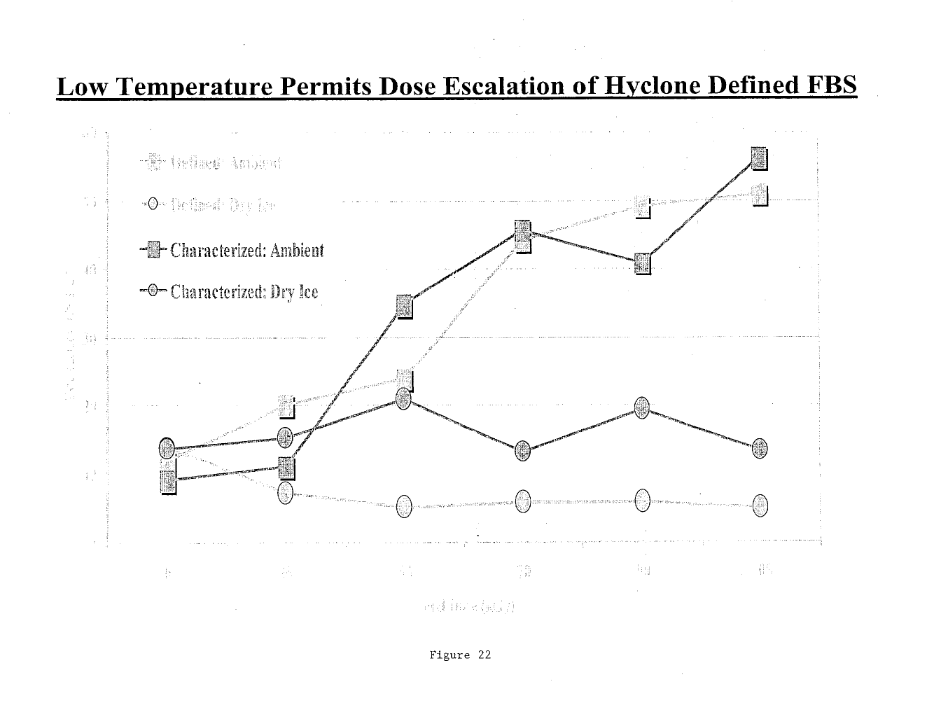

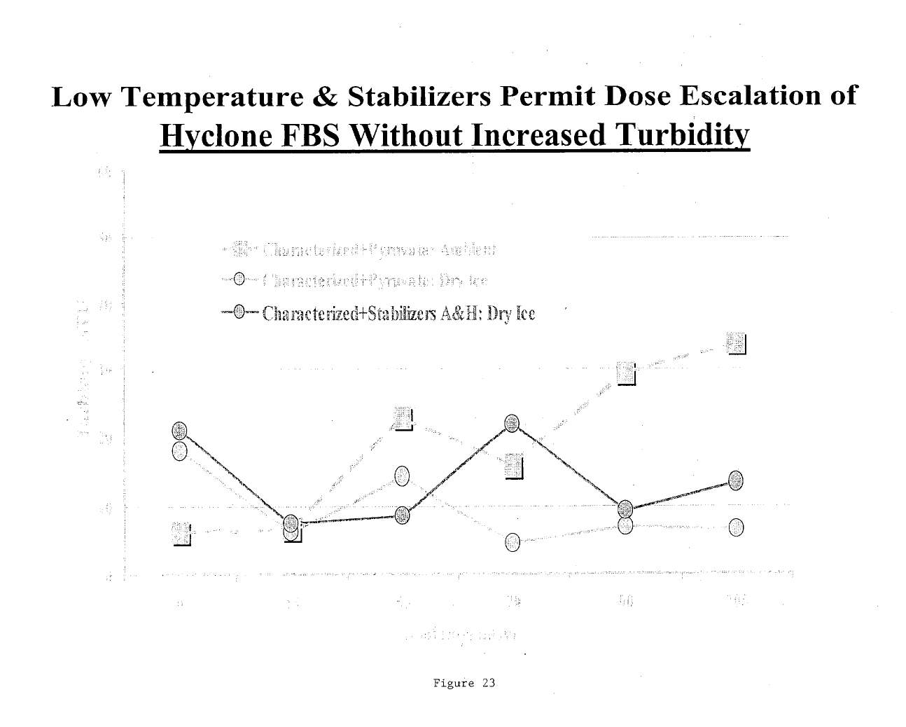

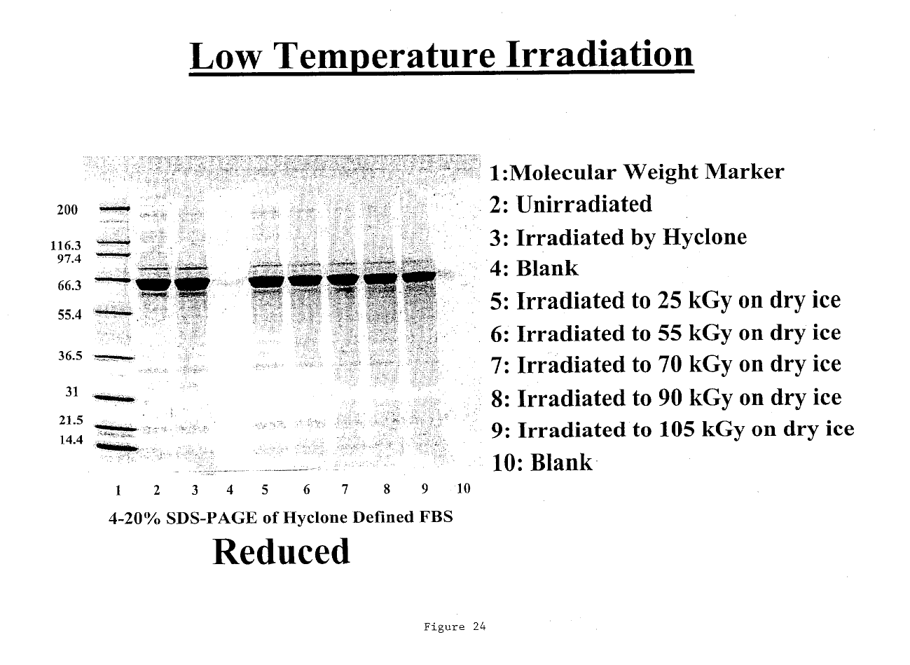

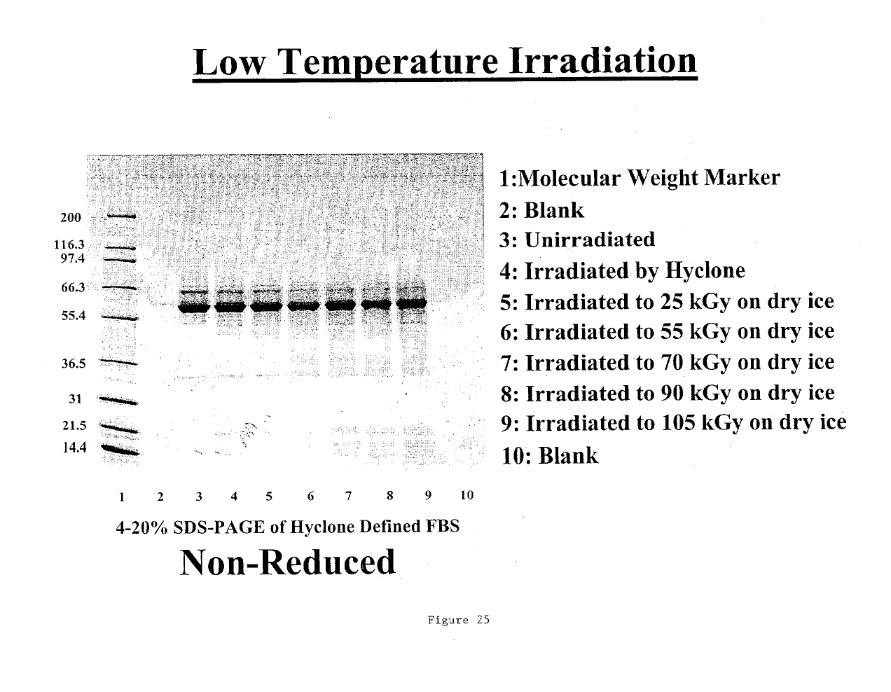

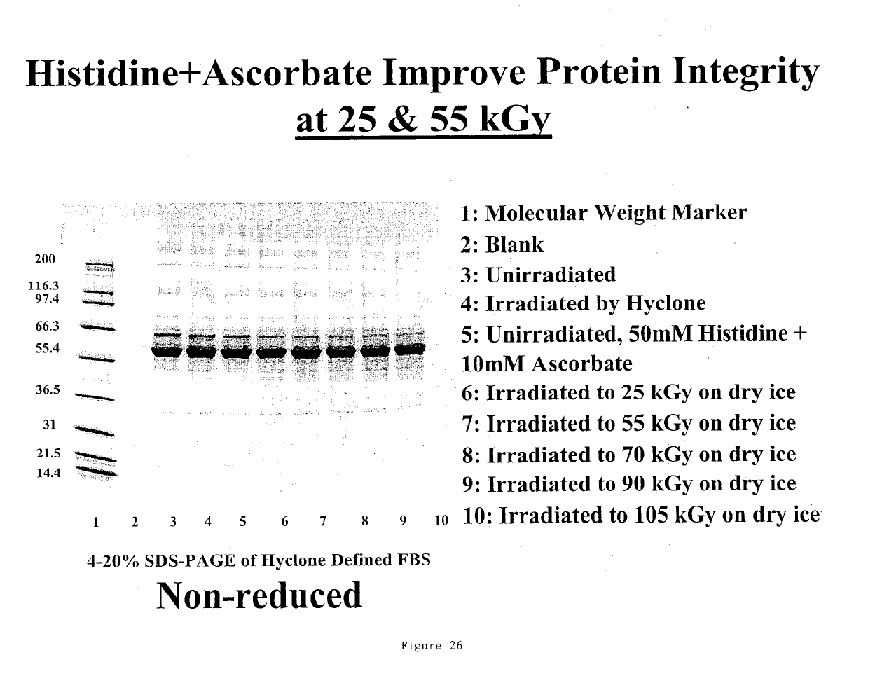

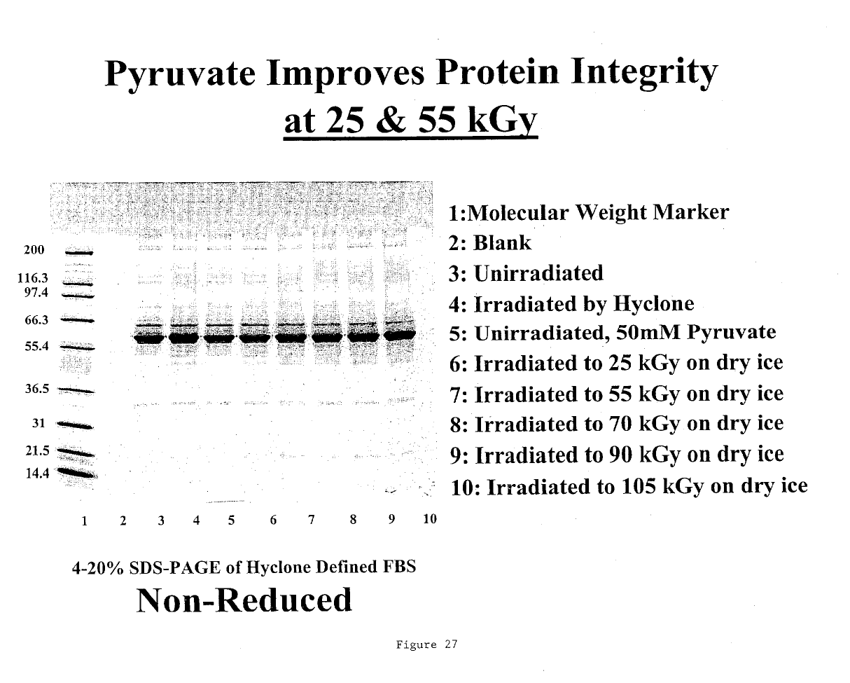

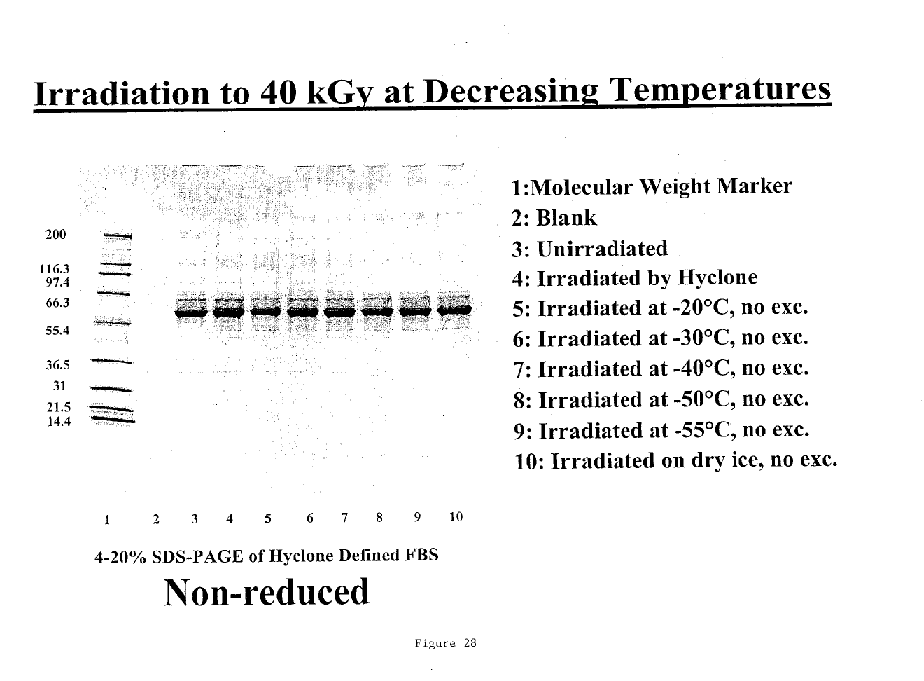

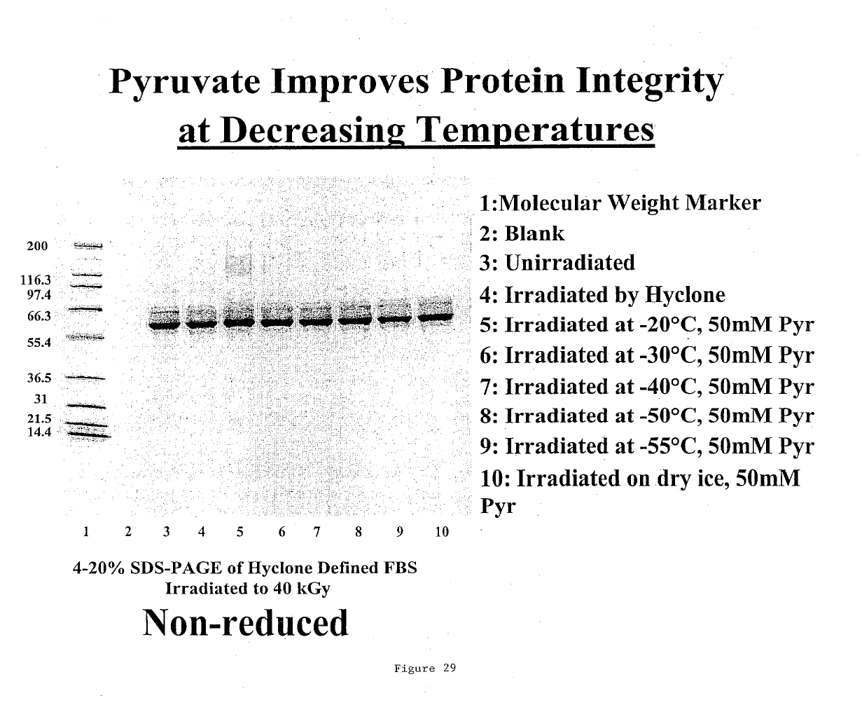

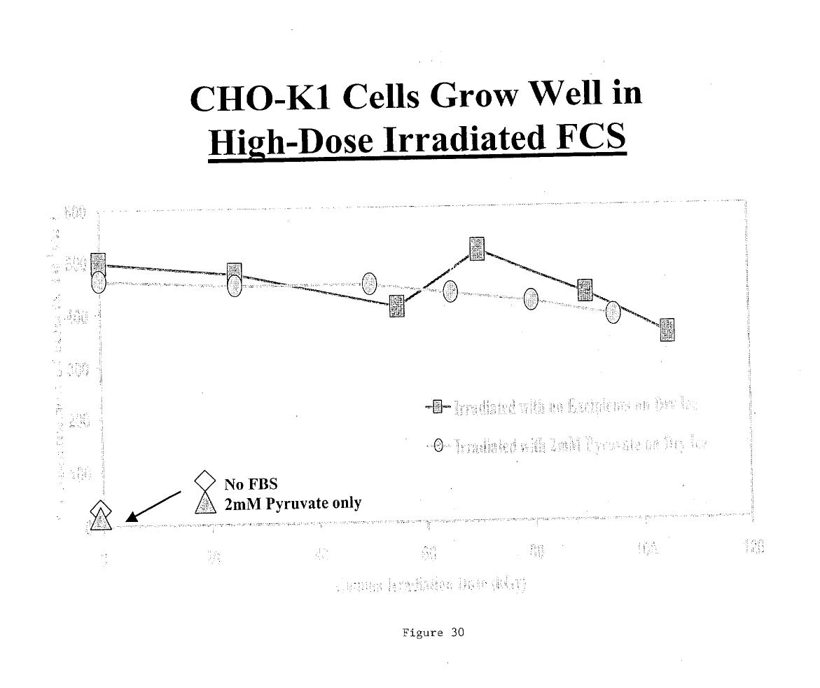

Results of experiments carried out in accordance with the guidelines of the above protocol or other preferred embodiments of the present invention are shown in FIGS. 1-30.

The foregoing embodiments and advantages are merely exemplary and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art. In the claims, means-plus-function clauses are intended to cover the structures described herein as performing the recited function and not only structural equivalents but also equivalent structures.

What is claimed is:

1. A method for sterilizing serum or plasma that is sensitive to radiation, said method comprising irradiating said serum or plasma with radiation for a time effective to sterilize said serum or plasma at a rate effective to sterilize said serum or plasma and to protect said serum or plasma from said radiation.

2. A method for sterilizing serum or plasma that is sensitive to radiation, said method comprising:
(i) applying to said serum or plasma at least one stabilizing process selected from the group consisting of:
(a) adding to said serum or plasma at least one stabilizer;
(b) reducing the residual solvent content of said serum or plasma;
(c) reducing the temperature of said serum or plasma;
(d) reducing the oxygen content of said serum or plasma;
(e) adjusting or maintaining the pH of said serum or plasma; and
(f) adding to said serum or plasma at least one non-aqueous solvent; and
(ii) irradiating said serum or plasma with a suitable radiation at an effective rate for a time effective to sterilize said serum or plasma, wherein said at least one stabilizing process protects said serum or plasma from said radiation.

3. A method for sterilizing serum or plasma that is sensitive to radiation, said method comprising irradiating said serum or plasma with radiation to a total dose effective to sterilize said serum or plasma at a rate effective to sterilize said serum or plasma and to protect said serum or plasma from said radiation.

4. The method according to claim 2, wherein said stabilizing process and said rate are together effective to protect said serum or plasma from said radiation.

5. The method according to claim 2, wherein at least two stabilizing processes are applied and said at least two stabilizing processes are together effective to protect said serum or plasma from said radiation.

6. The method according to claim 1, 2 or 3, wherein said effective rate is not more than 3.0 kGy/hour.

7. The method according to claim 1, 2 or 3, wherein said effective rate is not more than 2.5 kGy/hr.

8. The method according to claim 1, 2 or 3, wherein said effective rate is not mote than 2.0 kGy/hr.

9. The method according to claim 1, 2 or 3, wherein said effective rate is not more than 1.0 kGy/hr.

10. The method according to claim 1, 2 or 3, wherein said effective rate is not more than 0.3 kGy/hr.

11. The method according to claim 1, 2 or 3, wherein said effective rate is more than 3.0 kGy/hour.

12. The method according to claim 1, 2 or 3, wherein said effective rate is at least 5.0 kGy/hour.

13. The method according to claim 1, 2 or 3, wherein said effective rate is at least 18.0 kGy/hour.

14. The method according to claim 2, wherein said at least one stabilizer inhibits the generation of free radicals.

15. The method according to claim 2, wherein said at least one stabilizer inhibits the generation of reactive oxygen species.

16. The method according to claim 15, wherein said at least one stabilizer is an α-keto acid.

17. The method according to claim 16, wherein said at least one stabilizer is selected from the group consisting of pyruvate, lactate, derivatives and metabolites thereof and combinations thereof.

18. The method according to claim 2, wherein said at least one stabilizer comprises pyruvate.

19. The method according to claim 2, wherein said at least one stabilizer is selected from the group consisting of amino acids, vitamins, α-keto acids and combinations thereof.

20. The method according to claim 2, wherein said at least one stabilizer comprises histidine.

21. The method according to claim 2, wherein said at least one stabilizer comprises ascorbic acid or a salt or ester thereof

22. The method according to claim 2, wherein said at least one stabilizer is present in a concentration of at least 1 nM.

23. The method according to claim 2, wherein said at least one stabilizer is present in a concentration of at least 1 mM.

24. The method according to claim 2, wherein said at least one stabilizer is present in a concentration of at least 2 mM.

25. The method according to claim 2, wherein said at least one stabilizer is present in a concentration of at least 5 mM.

26. The method according to claim 2, wherein said at least one stabilizer is present in a concentration of at least 10 mM.

27. The method according to claim 2, wherein said at least one stabilizer is present in a concentration of at least 25 mM.

28. The method according to claim 2, wherein said at least one stabilizer is present in a concentration of at least 50 mM.

29. The method according to claim 2, wherein said at least one stabilizer is present in a concentration of at least 100 mM.

30. The method according to claim 2, wherein said at least one stabilizer is present in a concentration of from 0.1 mM to 10 mM.

31. The method according to claim 2, wherein said at least one stabilizer is present in a concentration of from 0.1 mM to 50 mM.

32. The method according to claim 2, wherein said at least one stabilizer is present in a concentration of from 10 mM to 100 mM.

33. The method according to claim 2, wherein said at least one stabilizer is present in a concentration of from 10 mM to 50 mM.

34. The method according to claim 2, wherein said at least one stabilizer is present in a concentration of from 50 mM to 100 mM.

35. The method according to claim 1, 2 or 3, wherein said serum or plasma contains pyruvate.

36. The method according to claim 35, wherein said pyruvate is present in a concentration of at least 50 mM.

37. The method according to claim 1, 2 or 3, wherein said serum or plasma contains histidine, ascorbic acid or a salt or ester thereof, or combinations thereof.

38. The method according to claim 37, wherein said histidine is present in a concentration of at least 100 mM.

39. The method according to claim 37, wherein said histidine is present in a concentration of at least 50 mM.

40. The method according to claim 37, wherein said ascorbic acid is in the form of an ascorbate salt and is present in a concentration of at least 100 mM.

41. The method according to claim 37, wherein said ascorbic acid is in the form of an ascorbate salt and is present in a concentration of at least 10 mM.

42. The method according to claim 37, wherein said ascorbic acid is in the form of an ascorbate salt and is present in a concentration of at least 0.1 mM.

43. The method according to claim 37, wherein said ascorbic acid is in the form of an ascorbate salt and is present in a concentration of at least 100 mM.

44. The method according to claim 37, wherein said ascorbic acid is in the form of an ascorbate salt and is present in a concentration of at least 10 mM.

45. The method according to claim 1, 2 or 3, wherein the turbidity of said serum or plasma after irradiation is less than 30 NTU.

46. The method according to claim 1, 2 or 3, wherein the turbidity of said serum or plasma after irradiation is less than 25 NTU.

47. The method according to claim 1, 2 or 3, wherein the turbidity of said serum or plasma after irradiation is less than 20 NTU.

48. The method according to claim 1, 2 or 3, wherein the turbidity of said serum or plasma after irradiation is less than 15 NTU.

49. The method according to claim 1, 2 or 3, wherein the turbidity of said serum or plasma after irradiation is less than 10 NTU.

50. The method according to claim 1, 2 or 3, wherein the turbidity of said serum or plasma after irradiation is less than 5 NTU.

51. The method according to claim 1, 2 or 3, wherein the turbidity of said serum or plasma after irradiation is less than 2.5 NTU.

52. The method according to claim 1, 2 or 3, wherein the turbidity of said serum or plasma is decreased following irradiation.

53. The method according to claim 1, 2 or 3, wherein the turbidity of said serum or plasma is not increased following irradiation.

54. The method according to claim 1, 2 or 3, wherein any increase in the turbidity of said serum or plasma following irradiation is less than the increase in the turbidity of serum or plasma following irradiation under different conditions.

55. The method according to claim 1, 2 or 3, wherein said radiation is corpuscular radiation, electromagnetic radiation, or a mixture thereof.

56. The method according to claim 55, wherein said electromagnetic radiation is selected from the group consisting of radio waves, microwaves, visible and invisible light, ultraviolet light, x-ray radiation, gamma radiation and combinations thereof.

57. The method according to claim 1, 2 or 3 wherein said radiation is gamma radiation.

58. The method according to claim 1, 2 or 3, wherein the temperature of said plasma or serum is about ambient temperature at the initiation of said irradiation.

59. The method according to claim 1, 2 or 3, wherein the temperature of said plasma or serum is below ambient temperature at the initiation of said irradiation.

60. The method according to claim 1, 2 or 3, wherein the temperature of said plasma or serum is below the freezing point of said plasma or serum at the initiation of said irradiation.

61. The method according to claim 1, 2 or 3, wherein the temperature of said plasma or serum is below the eutectic point of said plasma or serum at the initiation of said irradiation.

62. The method according to claim 1, 2 or 3, wherein the temperature of said plasma or serum is above ambient temperature at the initiation of said irradiation.

63. The method according to claim 1, 2 or 3, wherein the temperature of said plasma or serum is at least −75° C. at the initiation of said irradiation.

64. The method according to claim 1, 2 or 3, wherein the temperature of said plasma or serum is at least −70° C. at the initiation of said irradiation.

65. The method according to claim 1, 2 or 3, wherein the temperature of said plasma or serum is at least −55° C. at the initiation of said irradiation.

66. The method according to claim 1, 2 or 3, wherein the temperature of said plasma or serum is at least −50° C. at the initiation of said irradiation.

67. The method according to claim 1, 2 or 3, wherein the temperature of said plasma or serum is at least −40° C. at the initiation of said irradiation.

68. The method according to claim 1, 2 or 3, wherein the temperature of said plasma or serum is at least −30° C. at the initiation of said irradiation.

69. The method according to claim 1, 2 or 3, wherein the temperature of said plasma or serum is at least −20° C. at the initiation of said irradiation.

70. The method according to claim 1, 2 or 3, wherein the temperature of said plasma or serum is at least −10° C. at the initiation of said irradiation.

71. The method according to claim 1, 2 or 3, wherein the temperature of said plasma or serum is at least 4° C. at the initiation of said irradiation.

72. The method according to claim 1, 2 or 3, wherein the temperature of said plasma or serum is about the same temperature as dry ice at the initiation of said irradiation.

73. The method according to claim 1, 2 or 3, wherein the temperature of said plasma or serum is between −10° C. and −55° C. at the initiation of said irradiation.

74. The method according to claim 1, 2 or 3, wherein the temperature of said plasma or serum is between −10° C. and −50° C. at the initiation of said irradiation.

75. The method according to claim 1, 2 or 3, wherein the temperature of said plasma or serum is between −10° C. and −30° C. at the initiation of said irradiation.

76. The method according to claim 1, 2 or 3, wherein the temperature of said plasma or serum is between −30° C. and −70° C. at the initiation of said irradiation.

77. The method according to claim 1, 2 or 3, wherein the temperature of said plasma or serum is between −30° C. and −50° C. at the initiation of said irradiation.

78. The method according to claim 1, 2 or 3, wherein the temperature of said plasma or serum is between −40° C. and −55° C. at the initiation of said irradiation.

79. The method according to claim 1, 2 or 3, wherein the temperature of said plasma or serum is between −40° C. and −50° C. at the initiation of said irradiation.

80. The method according to claim 1, 2 or 3, wherein the functional activity of said serum or plasma after sterilization by irradiation is about 100% of the pre-irradiation value.

81. The method according to claim 1, 2 or 3, wherein the functional activity of said serum or plasma after sterilization by irradiation is greater than 100% of the pre-irradiation value.

82. The method according to claim 1, 2 or 3, wherein the functional activity of said serum or plasma after sterilization by irradiation is at least 95% of the pre-irradiation value.

83. The method according to claim 1, 2 or 3, wherein the functional activity of said serum or plasma after sterilization by irradiation is about 90% of the pre-irradiation value.

84. The method according to claim 1, 2 or 3, wherein the functional activity of said serum or plasma after sterilization by irradiation is about 85% of the pre-irradiation value.

85. The method according to claim 1, 2 or 3, wherein the functional activity of said serum or plasma after sterilization by irradiation is about 80% of the pre-irradiation value.

86. The method according to claim 1, 2 or 3, wherein the functional activity of said serum or plasma after sterilization by irradiation is about 70% of the pre-irradiation value.

87 The method according to claim 1, 2 or 3, wherein the functional activity of said serum or plasma after sterilization by irradiation is about 60% of the pre-irradiation value.

88. The method according to claim 1, 2 or 3, wherein the functional activity of said serum or plasma after sterilization by irradiation is about 50% of the pre-irradiation value.

89. The method according to claim 1, 2 or 3, wherein said serum or plasma is irradiated to a total dose of at least 105 kGy.

90. The method according to claim 1, 2 or 3, wherein said serum or plasma is irradiated to a total dose of at least 90 kGy.

91. The method according to claim 1, 2 or 3, wherein said serum or plasma is irradiated to a total dose of at least 80 kGy.

92. The method according to claim 1, 2 or 3, wherein said serum or plasma is irradiated to a total dose of at least 70 kGy.

93. The method according to claim 1, 2 or 3, wherein said serum or plasma is irradiated to a total dose of at least 65 kGy.

94. The method according to claim 1, 2 or 3, wherein said serum or plasma is irradiated to a total dose of at least 55 kGy.

95. The method according to claim 1, 2 or 3, wherein said serum or plasma is irradiated to a total dose of at least 50 kGy.

96. The method according to claim 1, 2 or 3, wherein said serum or plasma is irradiated to a total dose of at least 40 kGy.

97. The method according to claim 1, 2 or 3, wherein said serum or plasma is irradiated to a total dose of at least 30 kGy.

98. The method according to claim 1, 2 or 3, wherein said serum or plasma is irradiated to a total dose of at least 25 kGy.

99. The method according to claim 1, 2 or 3, wherein said serum or plasma is irradiated to a total dose of at least 10 kGy.

100. The method according to claim 2, wherein said serum or plasma has a pH of about 6.8 to about 8.1.

101. The method according to claim 2, wherein said serum or plasma has a pH of about 6.9 to about 7.4.

102. The method according to claim 2, wherein said serum or plasma has a pH of about 4.8 to about 5.5.

103. The method according to claim 1, 2 or 3, wherein said serum or plasma has less altered thermal stability up to at least 60° C. following irradiation than serum or plasma following irradiation under different conditions.

104. The method according to claim 1, 2 or 3, wherein said serum or plasma has less altered thermal stability up to at least 56° C. following irradiation than serum or plasma following irradiation under different conditions.

105. The method according to claim 1, 2 or 3, wherein said serum or plasma has less altered thermal stability up to at least 55° C. following irradiation than serum or plasma following irradiation under different conditions.

106. The method according to claim 1, 2 or 3, wherein said serum or plasma has less altered thermal stability up to at least 50° C. following irradiation than serum or plasma following irradiation under different conditions.

107. The method according to claim 1, 2 or 3, wherein said serum or plasma has less altered thermal stability up to at least 37° C. following irradiation than serum or plasma following irradiation under different conditions.

108. The method according to claim 1, 2 or 3, wherein said serum or plasma has less altered thermal stability up to at least 20° C. following irradiation than serum or plasma following irradiation under different conditions.

109. The method according to claim 1, 2 or 3, wherein said serum or plasma has less altered thermal stability up to at least 10° C. following irradiation than serum or plasma following irradiation under different conditions.

110. The method according to claim 1, 2 or 3, wherein said serum or plasma has less altered thermal stability up to at least 2° C. following irradiation than serum or plasma following irradiation under different conditions.

111. The method according to claim 1, 2 or 3, wherein said serum or plasma is fetal calf serum.

112. The method according to claim 1, 2 or 3, wherein said serum or plasma is human plasma.

113. The method according to claim 1, 2 or 3, wherein said serum or plasma is mammlian serum or plasma.

114. The method according to claim 1, 2 or 3, wherein said serum or plasma is bovine serum or plasma.

115. The method according to claim 1, 2 or 3, wherein said serum or plasma is ovine serum or plasma.

116. The method according to claim 1, 2 or 3, wherein said serum or plasma is porcine serum or plasma.

117. The method according to claim 1, 2 or 3, wherein said serum or plasma is equine serum or plasma.

118. The method according to claim 1, 2 or 3, wherein said serum or plasma is caprine serum or plasma.

119. The method according to claim 1, 2 or 3, wherein said serum or plasma is obtained from a fetal, immature or adult mammal.

120. The method according to claim 1, 2 or 3, wherein said serum or plasma contains at least one biological contaminant or pathogen selected from the group consisting of viruses, bacteria, yeasts, molds, fungi, parasites and prions or similar agents responsible, alone or in combination, for TSEs.

121. The method according to claim 1, 2 or 3, further comprising heating said serum or plasma to a temperature effective to alter one or more components of said serum or plasma.

122. The method according to claim 121, wherein said temperature is effective to reduce complement activity.

123. The method according to claim 121, wherein said temperature is effective to reduce the level of at least one active biological contaminant or pathogen.

124. The method according to claim 121, wherein said heating is performed subsequent to said irradiation.

125. The method according to claim 121, wherein said heating is performed prior to said irradiation.

126. The method according to claim 121, wherein said temperature is not less than 50° C.

127. The method according to claim 121, wherein said temperature is not less than 55° C.

128. The method according to claim 121, wherein said temperature is not less than 56° C.

129. The method according to claim 121, wherein said temperature is not less than 57° C.

130. The method according to claim 121, wherein said temperature is not less than 60° C.

131. The method according to claim 1, 2 or 3, wherein said effective rate is constant throughout said irradiation.

132. The method according to claim 1, 2 or 3, wherein said effective rate is not constant throughout said irradiation.

133. The method according to claim 132, wherein said effective rate is less than 3.0 kGy/hr for at least a portion of said irradiation.

134. The method according to claim 1, 2 or 3, wherein said irradiating is performed under conditions whereby the temperature of said serum or plasma increases during said irradiating from an initial temperature (Tᵢ) to a final temperature (T_{f}) and further wherein said increase in the temperature of said serum or plasma (A_{T}) is about equal to the total dose of said radiation (D) divided by the specific heat constant of said serum or plasma (c).

135. The method according to claim 134, wherein said final temperature (f) is at or below a temperature effective to protect said serum or plasma material from said radiation.

136. The method according to claim 134, wherein said increase in the temperature of said serum or plasma (ΔT) is about 0.25° C./kGy.

137. The method according to claim 2, wherein said residual solvent content is reduced by the addition of an effective amount of at least one solute.

138. The method according to claim 2, wherein said residual solvent content is reduced by lyophilization.

139. The method according to claim 1, 2 or 3, wherein the temperature of said plasma or serum is about ambient temperature for at least a portion of said irradiation.

140. The method according to claim 1, 2 or 3, wherein the temperature of said plasma or serum is below ambient temperature for at least a portion of said irradiation.

141. The method according to claim 1, 2 or 3, wherein the temperature of said plasma or serum is below the freezing point of said plasma or serum for at least a portion of said irradiation.

142. The method according to claim 1, 2 or 3, wherein the temperature of said plasma or serum is below the eutectic point of said plasma or serum for at least a portion of said irradiation.

143. The method according to claim 1, 2 or 3, wherein the temperature of said plasma or serum is above ambient temperature for at least a portion of said irradiation.

144. The method according to claim 1, 2 or 3, wherein the temperature of said plasma or serum is at least −75° C. for at least a portion of said irradiation.

145. The method according to claim 1, 2 or 3, wherein the temperature of said plasma or serum is at least −70° C. for at least a portion of said irradiation.

146. The method according to claim 1, 2 or 3, wherein the temperature of said plasma or serum is at least −55° C. for at least a portion of said irradiation.

147. The method according to claim 1, 2 or 3, wherein the temperature of said plasma or serum is at least −50° C. for at least a portion of said irradiation.

148. The method according to claim 1, 2 or 3, wherein the temperature of said plasma or serum is at least −40° C. for at least a portion of said irradiation.

149. The method according to claim 1, 2 or 3, wherein the temperature of said plasma or serum is at least −30° C. for at least a portion of said irradiation.

150. The method according to claim 1, 2 or 3, wherein the temperature of said plasma or serum is at least −20° C. for at least a portion of said irradiation.

151. The method according to claim 1, 2 or 3, wherein the temperature of said plasma or serum is at least −10° C. for at least a portion of said irradiation.

152. The method according to claim 1, 2 or 3, wherein the temperature of said plasma or serum is at least 4° C. for at least a portion of said irradiation.

153. The method according to claim 1, 2 or 3, wherein the temperature of said plasma or serum is about the same temperature as dry ice for at least a portion of said irradiation.

154. The method according to claim 1, 2 or 3, wherein the temperature of said plasma or serum is between −10° C. and −55° C. for at least a portion of said irradiation.

155. The method according to claim 1, 2 or 3, wherein the temperature of said plasma or serum is between −10° C. and −50° C. for at least a portion of said irradiation.

156. The method according to claim 1, 2 or 3, wherein the temperature of said plasma or serum is between −10° C. and −30° C. for at least a portion of said irradiation.

157. The method according to claim 1, 2 or 3, wherein the temperature of said plasma or serum is between −30° C. and −70° C. for at least a portion of said irradiation.

158. The method according to claim 1, 2 or 3, wherein the temperature of said plasma or serum is between −30° C. and −50° C. for at least a portion of said irradiation.

159. The method according to claim 1, 2 or 3, wherein the temperature of said plasma or serum is between −40° C. and −55° C. for at least a portion of said irradiation.

160. The method according to claim 1, 2 or 3, wherein the temperature of said plasma or serum is between −40° C. and −50° C. for at least a portion of said irradiation.

161. The method according to claim 2, wherein said residual solvent content of said serum or plasma is reduced to a level of less than 5.0% prior to said irradiation.

162. The method according to claim 2, wherein said residual solvent content of said serum or plasma is reduced to a level of less than 3.0% prior to said irradiation.

163. The method according to claim 2, wherein said residual solvent content of said serum or plasma is reduced to a level of less than 2.5% prior to said irradiation.

164. The method according to claim 2, wherein said residual solvent content of said serum or plasma is reduced to a level of less than 2.0% prior to said irradiation.

165. The method according to claim 2, wherein said residual solvent content of said serum or plasma is reduced to a level of less than 1.0% prior to said irradiation.

166. The method according to claim 2, wherein said residual solvent content of said serum or plasma is reduced to a level of less than 0.5% prior to said irradiation.

167. The method according to claim 2, wherein said residual solvent content of said serum or plasma is reduced to a level of less than 0.3% prior to said irradiation.

168. The method according to claim 2, wherein said residual solvent content of said serum or plasma is reduced to a level of less than 0.2% prior to said irradiation.

169. The method according to claim 2, wherein said residual solvent content of said serum or plasma is reduced to a level between 0.2% and 10.0% prior to said irradiation.

170. The method according to claim 2, wherein said residual solvent content of said serum or plasma is reduced to a level between 0.5% and 5.0% prior to said irradiation.

171. The method according to claim 2, wherein said residual solvent content of said serum or plasma is reduced to a level between 0.5% and 2.5% prior to said irradiation.

172. The method according to claim 2, wherein said residual solvent content of said serum or plasma is reduced to a level between 1.0% and 10.0% prior to said irradiation.

173. The method according to claim 2, wherein said residual solvent content of said serum or plasma is reduced to a level between 2.5% and 10.0% prior to said irradiation.

174. The method according to claim 137, wherein said solute is mannitol or ascorbic acid, or a salt or ester thereof, or combinations thereof.
